SEQUENCE LISTING (807 sequences)

SEQ ID NO: 1 - DNA, 26 bp, Artificial
  Oligonucleotide
cacttgtgccctgactttcaactctg

SEQ ID NO: 2 - DNA, 26 bp, Artificial
  Oligonucleotide
agtgaatctgaggcataactgcaccc

SEQ ID NO: 3 - DNA, 10 bp, Artificial
  Oligonucleotide
gttgctgtcg

SEQ ID NO: 4 - DNA, 11 bp, Artificial
  Oligonucleotide
ataccgatgca

SEQ ID NO: 5 - DNA, 17 bp, Artificial
  Oligonucleotide
caggaaacagctatgac

SEQ ID NO: 6 - DNA, 10 bp, Artificial
  Oligonucleotide
attggtgctg

SEQ ID NO: 7 - DNA, 10 bp, Artificial
  Oligonucleotide
cgtccnnncc

SEQ ID NO: 8 - DNA, 10 bp, Artificial
  Oligonucleotide
cgtccnnccc

SEQ ID NO: 9 - DNA, 10 bp, Artificial
  Oligonucleotide
cgtccnngcc

SEQ ID NO: 10 - DNA, 10 bp, Artificial
  Oligonucleotide
cgtccnntcc

SEQ ID NO: 11 - DNA, 10 bp, Artificial
  Oligonucleotide
cgtccncncc

SEQ ID NO: 12 - DNA, 10 bp, Artificial
  Oligonucleotide
cgtccncccc

SEQ ID NO: 13 - DNA, 10 bp, Artificial
  Oligonucleotide
cgtccacgcc

SEQ ID NO: 14 - DNA, 10 bp, Artificial
  Oligonucleotide
cgtccnctcc

SEQ ID NO: 15 - DNA, 10 bp, Artificial
  Oligonucleotide
cgtccngncc

SEQ ID NO: 16 - DNA, 10 bp, Artificial
  Oligonucleotide
cgtccagccc

SEQ ID NO: 17 - DNA, 10 bp, Artificial
  Oligonucleotide
cgtccaggcc

SEQ ID NO: 18 - DNA, 10 bp, Artificial
  Oligonucleotide
cgtccngtcc

SEQ ID NO: 19 - DNA, 10 bp, Artificial
  Oligonucleotide
cgtccntncc

SEQ ID NO: 20 - DNA, 10 bp, Artificial
  Oligonucleotide
cgtccntccc

SEQ ID NO: 21 - DNA, 10 bp, Artificial
  Oligonucleotide
cgtccntgcc

SEQ ID NO: 22 - DNA, 10 bp, Artificial
  Oligonucleotide
cgtccnttcc

SEQ ID NO: 23 - DNA, 10 bp, Artificial
  Oligonucleotide
cgtcccnncc

SEQ ID NO: 24 - DNA, 10 bp, Artificial
  Oligonucleotide
cgtcccnccc

SEQ ID NO: 25 - DNA, 10 bp, Artificial
  Oligonucleotide
cgtcccagcc

SEQ ID NO: 26 - DNA, 10 bp, Artificial
  Oligonucleotide
cgtcccntcc

SEQ ID NO: 27 - DNA, 10 bp, Artificial
  Oligonucleotide
cgtccccacc

SEQ ID NO: 28 - DNA, 10 bp, Artificial
  Oligonucleotide
cgtccccccc

SEQ ID NO: 29 - DNA, 10 bp, Artificial
  Oligonucleotide
cgtccccgcc

SEQ ID NO: 30 - DNA, 10 bp, Artificial
  Oligonucleotide
cgtcccctcc

SEQ ID NO: 31 - DNA, 10 bp, Artificial
  Oligonucleotide
cgtcccgncc

SEQ ID NO: 32 - DNA, 10 bp, Artificial
  Oligonucleotide
cgtcccgccc

SEQ ID NO: 33 - DNA, 10 bp, Artificial
  Oligonucleotide
cgtcccggcc

SEQ ID NO: 34 - DNA, 10 bp, Artificial
  Oligonucleotide
cgtcccgtcc

SEQ ID NO: 35 - DNA, 10 bp, Artificial
  Oligonucleotide
cgtcccntcc

SEQ ID NO: 36 - DNA, 10 bp, Artificial
  Oligonucleotide
cgtccctccc

SEQ ID NO: 37 - DNA, 10 bp, Artificial
  Oligonucleotide
cgtccctgcc

SEQ ID NO: 38 - DNA, 10 bp, Artificial
  Oligonucleotide
cgtcccttcc

SEQ ID NO: 39 - DNA, 10 bp, Artificial
  Oligonucleotide
cgtccgnncc

SEQ ID NO: 40 - DNA, 10 bp, Artificial
  Oligonucleotide
cgtccgnccc

SEQ ID NO: 41 - DNA, 10 bp, Artificial
  Oligonucleotide
cgtccgngcc

SEQ ID NO: 42 - DNA, 10 bp, Artificial
  Oligonucleotide
cgtccgntcc

SEQ ID NO: 43 - DNA, 10 bp, Artificial
  Oligonucleotide
cgtccgcncc

SEQ ID NO: 44 - DNA, 10 bp, Artificial
  Oligonucleotide
cgtccgcccc

SEQ ID NO: 45 - DNA, 10 bp, Artificial
  Oligonucleotide
cgtccgcgcc

SEQ ID NO: 46 - DNA, 10 bp, Artificial
  Oligonucleotide
cgtccgctcc

SEQ ID NO: 47 - DNA, 10 bp, Artificial
  Oligonucleotide
cgtccgctcc

SEQ ID NO: 48 - DNA, 10 bp, Artificial
  Oligonucleotide
cgtccggncc

SEQ ID NO: 49 - DNA, 10 bp, Artificial
  Oligonucleotide
cgtccggccc

SEQ ID NO: 50 - DNA, 10 bp, Artificial
  Oligonucleotide
cgtccgggcc

SEQ ID NO: 51 - DNA, 10 bp, Artificial
  Oligonucleotide
cgtccggtcc

SEQ ID NO: 52 - DNA, 10 bp, Artificial
  Oligonucleotide
cgtccgtncc

SEQ ID NO: 53 - DNA, 10 bp, Artificial
  Oligonucleotide
cgtccgtccc

SEQ ID NO: 54 - DNA, 10 bp, Artificial
  Oligonucleotide
cgtccgtgcc

SEQ ID NO: 55 - DNA, 10 bp, Artificial
  Oligonucleotide
cgtccgttcc

SEQ ID NO: 56 - DNA, 10 bp, Artificial
  Oligonucleotide
cgtcctnncc

SEQ ID NO: 57 - DNA, 10 bp, Artificial
  Oligonucleotide
cgtcctnccc

SEQ ID NO: 58 - DNA, 10 bp, Artificial
  Oligonucleotide
cgtcctngcc

SEQ ID NO: 59 - DNA, 10 bp, Artificial
  Oligonucleotide
cgtcctntcc

SEQ ID NO: 60 - DNA, 10 bp, Artificial
  Oligonucleotide
cgtcctcncc

SEQ ID NO: 61 - DNA, 10 bp, Artificial
  Oligonucleotide
cgtcctcccc

SEQ ID NO: 62 - DNA, 10 bp, Artificial
  Oligonucleotide
cgtcctcgcc

SEQ ID NO: 63 - DNA, 10 bp, Artificial
  Oligonucleotide
cgtcctctcc

SEQ ID NO: 64 - DNA, 10 bp, Artificial
  Oligonucleotide
cgtcctgncc

SEQ ID NO: 65 - DNA, 10 bp, Artificial
  Oligonucleotide
cgtcctgccc

SEQ ID NO: 66 - DNA, 10 bp, Artificial
  Oligonucleotide
cgtcctggcc

SEQ ID NO: 67 - DNA, 10 bp, Artificial
  Oligonucleotide
cgtcctgtcc

SEQ ID NO: 68 - DNA, 10 bp, Artificial
  Oligonucleotide
cgtccttncc

SEQ ID NO: 69 - DNA, 10 bp, Artificial
  Oligonucleotide
cgtccttccc

SEQ ID NO: 70 - DNA, 10 bp, Artificial
  Oligonucleotide
cgtccttgcc

SEQ ID NO: 71 - DNA, 10 bp, Artificial
  Oligonucleotide
cgtcctttcc

SEQ ID NO: 72 - DNA, 10 bp, Artificial
  Oligonucleotide
cgtccnnncg

SEQ ID NO: 73 - DNA, 10 bp, Artificial
  Oligonucleotide
cgtccnnccg

SEQ ID NO: 74 - DNA, 10 bp, Artificial
  Oligonucleotide
cgtccnngcg

SEQ ID NO: 75 - DNA, 10 bp, Artificial
  Oligonucleotide
cgtccnntcg

SEQ ID NO: 76 - DNA, 10 bp, Artificial
  Oligonucleotide
cgtccncncg

SEQ ID NO: 77 - DNA, 10 bp, Artificial
  Oligonucleotide
cgtccncccg

SEQ ID NO: 78 - DNA, 10 bp, Artificial
  Oligonucleotide
cgtccncgcg

SEQ ID NO: 79 - DNA, 10 bp, Artificial
  Oligonucleotide
cgtccnctcg

SEQ ID NO: 80 - DNA, 10 bp, Artificial
  Oligonucleotide
cgtccngncg

SEQ ID NO: 81 - DNA, 10 bp, Artificial
  Oligonucleotide
cgtccngccg

SEQ ID NO: 82 - DNA, 10 bp, Artificial
  Oligonucleotide
cgtccnggcg

SEQ ID NO: 83 - DNA, 10 bp, Artificial
  Oligonucleotide
cgtccngtcg

SEQ ID NO: 84 - DNA, 10 bp, Artificial
  Oligonucleotide
cgtccntncg

SEQ ID NO: 85 - DNA, 10 bp, Artificial
  Oligonucleotide
cgtccntccg

SEQ ID NO: 86 - DNA, 10 bp, Artificial
  Oligonucleotide
cgtccntgcg

SEQ ID NO: 87 - DNA, 10 bp, Artificial
  Oligonucleotide
cgtccnttcg

SEQ ID NO: 88 - DNA, 10 bp, Artificial
  Oligonucleotide
cgtcccnncg

SEQ ID NO: 89 - DNA, 10 bp, Artificial
  Oligonucleotide
cgtcccnccg

SEQ ID NO: 90 - DNA, 10 bp, Artificial
  Oligonucleotide
cgtcccngcg

SEQ ID NO: 91 - DNA, 10 bp, Artificial
  Oligonucleotide
cgtcccntcg

SEQ ID NO: 92 - DNA, 10 bp, Artificial
  Oligonucleotide
cgtccccncg

SEQ ID NO: 93 - DNA, 10 bp, Artificial
  Oligonucleotide
cgtccccccg

SEQ ID NO: 94 - DNA, 10 bp, Artificial
  Oligonucleotide
cgtccccgcg

SEQ ID NO: 95 - DNA, 10 bp, Artificial
  Oligonucleotide
cgtcccctcg

SEQ ID NO: 96 - DNA, 10 bp, Artificial
  Oligonucleotide
cgtcccgncg

SEQ ID NO: 97 - DNA, 10 bp, Artificial
  Oligonucleotide
cgtcccgccg

SEQ ID NO: 98 - DNA, 10 bp, Artificial
  Oligonucleotide
cgtcccggcg

SEQ ID NO: 99 - DNA, 10 bp, Artificial
  Oligonucleotide
cgtcccgtcg

SEQ ID NO: 100 - DNA, 10 bp, Artificial
  Oligonucleotide
cgtccctncg

SEQ ID NO: 101 - DNA, 10 bp, Artificial
  Oligonucleotide
cgtccctccg

SEQ ID NO: 102 - DNA, 10 bp, Artificial
  Oligonucleotide
cgtccctgcg

SEQ ID NO: 103 - DNA, 10 bp, Artificial
  Oligonucleotide
cgtcccttcg

SEQ ID NO: 104 - DNA, 10 bp, Artificial
  Oligonucleotide
cgtccgnncg

SEQ ID NO: 105 - DNA, 10 bp, Artificial
  Oligonucleotide
cgtccgnccg

SEQ ID NO: 106 - DNA, 10 bp, Artificial
  Oligonucleotide
cgtccgagcg

SEQ ID NO: 107 - DNA, 10 bp, Artificial
  Oligonucleotide
cgtccgntcg

SEQ ID NO: 108 - DNA, 10 bp, Artificial
  Oligonucleotide
cgtccgcncg

SEQ ID NO: 109 - DNA, 10 bp, Artificial
  Oligonucleotide
cgtccgcccg

SEQ ID NO: 110 - DNA, 10 bp, Artificial
  Oligonucleotide
cgtccgcgcg

SEQ ID NO: 111 - DNA, 10 bp, Artificial
  Oligonucleotide
cgtccgctcg

SEQ ID NO: 112 - DNA, 10 bp, Artificial
  Oligonucleotide
cgtccggncg

SEQ ID NO: 113 - DNA, 10 bp, Artificial
  Oligonucleotide
cgtccggccg

SEQ ID NO: 114 - DNA, 10 bp, Artificial
  Oligonucleotide
cgtccgggcg

SEQ ID NO: 115 - DNA, 10 bp, Artificial
  Oligonucleotide
cgtccggtcg

SEQ ID NO: 116 - DNA, 10 bp, Artificial
  Oligonucleotide
cgtccgtncg

SEQ ID NO: 117 - DNA, 10 bp, Artificial
  Oligonucleotide
cgtccgtccg

SEQ ID NO: 118 - DNA, 10 bp, Artificial
  Oligonucleotide
cgtccgtgcg

SEQ ID NO: 119 - DNA, 10 bp, Artificial
  Oligonucleotide
cgtccgttcg

SEQ ID NO: 120 - DNA, 10 bp, Artificial
  Oligonucleotide
cgtcctnncg

SEQ ID NO: 121 - DNA, 10 bp, Artificial
  Oligonucleotide
cgtcctnccg

SEQ ID NO: 122 - DNA, 10 bp, Artificial
  Oligonucleotide
cgtcctngcg

SEQ ID NO: 123 - DNA, 10 bp, Artificial
  Oligonucleotide
cgtcctntcg

SEQ ID NO: 124 - DNA, 10 bp, Artificial
  Oligonucleotide
cgtcctcncg

SEQ ID NO: 125 - DNA, 10 bp, Artificial
  Oligonucleotide
cgtcctcccg

SEQ ID NO: 126 - DNA, 10 bp, Artificial
  Oligonucleotide
cgtcctcgcg

SEQ ID NO: 127 - DNA, 10 bp, Artificial
  Oligonucleotide
cgtcctctcg

SEQ ID NO: 128 - DNA, 10 bp, Artificial
  Oligonucleotide
cgtcctgncg

SEQ ID NO: 129 - DNA, 10 bp, Artificial
  Oligonucleotide
cgtcctgccg

SEQ ID NO: 130 - DNA, 10 bp, Artificial
  Oligonucleotide
cgtcctggcg

SEQ ID NO: 131 - DNA, 10 bp, Artificial
  Oligonucleotide
cgtcctgtcg

SEQ ID NO: 132 - DNA, 10 bp, Artificial
  Oligonucleotide
cgtccttncg

SEQ ID NO: 133 - DNA, 10 bp, Artificial
  Oligonucleotide
cgtccttccg

SEQ ID NO: 134 - DNA, 10 bp, Artificial
  Oligonucleotide
cgtccttgcg

SEQ ID NO: 135 - DNA, 10 bp, Artificial
  Oligonucleotide
cgtcctttcg

SEQ ID NO: 136 - DNA, 10 bp, Artificial
  Oligonucleotide
cgtccnnngc

SEQ ID NO: 137 - DNA, 10 bp, Artificial
  Oligonucleotide
cgtccnncgc

SEQ ID NO: 138 - DNA, 10 bp, Artificial
  Oligonucleotide
cgtccnnggc

SEQ ID NO: 139 - DNA, 10 bp, Artificial
  Oligonucleotide
cgtccnntgc

SEQ ID NO: 140 - DNA, 10 bp, Artificial
  Oligonucleotide
cgtccncngc

SEQ ID NO: 141 - DNA, 10 bp, Artificial
  Oligonucleotide
cgtccnccgc

SEQ ID NO: 142 - DNA, 10 bp, Artificial
  Oligonucleotide
cgtccncggc

SEQ ID NO: 143 - DNA, 10 bp, Artificial
  Oligonucleotide
cgtccnctgc

SEQ ID NO: 144 - DNA, 10 bp, Artificial
  Oligonucleotide
cgtccngngc

SEQ ID NO: 145 - DNA, 10 bp, Artificial
  Oligonucleotide
cgtccngcgc

SEQ ID NO: 146 - DNA, 10 bp, Artificial
  Oligonucleotide
cgtccngggc

SEQ ID NO: 147 - DNA, 10 bp, Artificial
  Oligonucleotide
cgtccngtgc

SEQ ID NO: 148 - DNA, 10 bp, Artificial
  Oligonucleotide
cgtccntngc

SEQ ID NO: 149 - DNA, 10 bp, Artificial
  Oligonucleotide
cgtccntcgc

SEQ ID NO: 150 - DNA, 10 bp, Artificial
  Oligonucleotide
cgtccntggc

SEQ ID NO: 151 - DNA, 10 bp, Artificial
  Oligonucleotide
cgtccnttgc

SEQ ID NO: 152 - DNA, 10 bp, Artificial
  Oligonucleotide
cgtcccnngc

SEQ ID NO: 153 - DNA, 10 bp, Artificial
  Oligonucleotide
cgtcccncgc

SEQ ID NO: 154 - DNA, 10 bp, Artificial
  Oligonucleotide
cgtcccnggc

SEQ ID NO: 155 - DNA, 10 bp, Artificial
  Oligonucleotide
cgtcccntgc

SEQ ID NO: 156 - DNA, 10 bp, Artificial
  Oligonucleotide
cgtccccngc

SEQ ID NO: 157 - DNA, 10 bp, Artificial
  Oligonucleotide
cgtcccccgc

SEQ ID NO: 158 - DNA, 10 bp, Artificial
  Oligonucleotide
cgtccccggc

SEQ ID NO: 159 - DNA, 10 bp, Artificial
  Oligonucleotide
cgtcccctgc

SEQ ID NO: 160 - DNA, 10 bp, Artificial
  Oligonucleotide
cgtcccgngc

SEQ ID NO: 161 - DNA, 10 bp, Artificial
  Oligonucleotide
cgtcccgcgc

SEQ ID NO: 162 - DNA, 10 bp, Artificial
  Oligonucleotide
cgtcccgggc

SEQ ID NO: 163 - DNA, 10 bp, Artificial
  Oligonucleotide
cgtcccgtgc

SEQ ID NO: 164 - DNA, 10 bp, Artificial
  Oligonucleotide
cgtccctngc

SEQ ID NO: 165 - DNA, 10 bp, Artificial
  Oligonucleotide
cgtccctcgc

SEQ ID NO: 166 - DNA, 10 bp, Artificial
  Oligonucleotide
cgtccctggc

SEQ ID NO: 167 - DNA, 10 bp, Artificial
  Oligonucleotide
cgtcccttgc

SEQ ID NO: 168 - DNA, 10 bp, Artificial
  Oligonucleotide
cgtccgnngc

SEQ ID NO: 169 - DNA, 10 bp, Artificial
  Oligonucleotide
cgtccgncgc

SEQ ID NO: 170 - DNA, 10 bp, Artificial
  Oligonucleotide
cgtccgnggc

SEQ ID NO: 171 - DNA, 10 bp, Artificial
  Oligonucleotide
cgtccgntgc

SEQ ID NO: 172 - DNA, 10 bp, Artificial
  Oligonucleotide
cgtccgcngc

SEQ ID NO: 173 - DNA, 10 bp, Artificial
  Oligonucleotide
cgtccgccgc

SEQ ID NO: 174 - DNA, 10 bp, Artificial
  Oligonucleotide
cgtccgcggc

SEQ ID NO: 175 - DNA, 10 bp, Artificial
  Oligonucleotide
cgtccgctgc

SEQ ID NO: 176 - DNA, 10 bp, Artificial
  Oligonucleotide
cgtccggngc

SEQ ID NO: 177 - DNA, 10 bp, Artificial
  Oligonucleotide
cgtccggcgc

SEQ ID NO: 178 - DNA, 10 bp, Artificial
  Oligonucleotide
cgtccggggc

SEQ ID NO: 179 - DNA, 10 bp, Artificial
  Oligonucleotide
cgtccggtgc

SEQ ID NO: 180 - DNA, 10 bp, Artificial
  Oligonucleotide
cgtccgtngc

SEQ ID NO: 181 - DNA, 10 bp, Artificial
  Oligonucleotide
cgtccgtcgc

SEQ ID NO: 182 - DNA, 10 bp, Artificial
  Oligonucleotide
cgtccgtggc

SEQ ID NO: 183 - DNA, 10 bp, Artificial
  Oligonucleotide
cgtccgttgc

SEQ ID NO: 184 - DNA, 10 bp, Artificial
  Oligonucleotide
cgtcctnngc

SEQ ID NO: 185 - DNA, 10 bp, Artificial
  Oligonucleotide
cgtcctncgc

SEQ ID NO: 186 - DNA, 10 bp, Artificial
  Oligonucleotide
cgtcctnggc

SEQ ID NO: 187 - DNA, 10 bp, Artificial
  Oligonucleotide
cgtcctntgc

SEQ ID NO: 188 - DNA, 10 bp, Artificial
  Oligonucleotide
cgtcctcngc

SEQ ID NO: 189 - DNA, 10 bp, Artificial
  Oligonucleotide
cgtcctccgc

SEQ ID NO: 190 - DNA, 10 bp, Artificial
  Oligonucleotide
cgtcctcggc

SEQ ID NO: 191 - DNA, 10 bp, Artificial
  Oligonucleotide
cgtcctctgc

SEQ ID NO: 192 - DNA, 10 bp, Artificial
  Oligonucleotide
cgtcctgngc

SEQ ID NO: 193 - DNA, 10 bp, Artificial
  Oligonucleotide
cgtcctgcgc

SEQ ID NO: 194 - DNA, 10 bp, Artificial
  Oligonucleotide
cgtcctgggc

SEQ ID NO: 195 - DNA, 10 bp, Artificial
  Oligonucleotide
cgtcctgtgc

SEQ ID NO: 196 - DNA, 10 bp, Artificial
  Oligonucleotide
cgtccttngc

SEQ ID NO: 197 - DNA, 10 bp, Artificial
  Oligonucleotide
cgtccttcgc

SEQ ID NO: 198 - DNA, 10 bp, Artificial
  Oligonucleotide
cgtccttggc

SEQ ID NO: 199 - DNA, 10 bp, Artificial
  Oligonucleotide
cgtcctttgc

SEQ ID NO: 200 - DNA, 10 bp, Artificial
  Oligonucleotide
cgtccnnngg

SEQ ID NO: 201 - DNA, 10 bp, Artificial
  Oligonucleotide
cgtccnncgg

SEQ ID NO: 202 - DNA, 10 bp, Artificial
  Oligonucleotide
cgtccnnggg

SEQ ID NO: 203 - DNA, 10 bp, Artificial
  Oligonucleotide
cgtccnntgg

SEQ ID NO: 204 - DNA, 10 bp, Artificial
  Oligonucleotide
cgtccncngg

SEQ ID NO: 205 - DNA, 10 bp, Artificial
  Oligonucleotide
cgtccaccgg

SEQ ID NO: 206 - DNA, 10 bp, Artificial
  Oligonucleotide
cgtccacggg

SEQ ID NO: 207 - DNA, 10 bp, Artificial
  Oligonucleotide
cgtccnctgg

SEQ ID NO: 208 - DNA, 10 bp, Artificial
  Oligonucleotide
cgtccngngg

SEQ ID NO: 209 - DNA, 10 bp, Artificial
  Oligonucleotide
cgtccagcgg

SEQ ID NO: 210 - DNA, 10 bp, Artificial
  Oligonucleotide
cgtccagggg

SEQ ID NO: 211 - DNA, 10 bp, Artificial
  Oligonucleotide
cgtccngtgg

SEQ ID NO: 212 - DNA, 10 bp, Artificial
  Oligonucleotide
cgtccntngg

SEQ ID NO: 213 - DNA, 10 bp, Artificial
  Oligonucleotide
cgtccntcgg

SEQ ID NO: 214 - DNA, 10 bp, Artificial
  Oligonucleotide
cgtccntggg

SEQ ID NO: 215 - DNA, 10 bp, Artificial
  Oligonucleotide
cgtccnttgg

SEQ ID NO: 216 - DNA, 10 bp, Artificial
  Oligonucleotide
cgtcccnngg

SEQ ID NO: 217 - DNA, 10 bp, Artificial
  Oligonucleotide
cgtcccncgg

SEQ ID NO: 218 - DNA, 10 bp, Artificial
  Oligonucleotide
cgtcccaggg

SEQ ID NO: 219 - DNA, 10 bp, Artificial
  Oligonucleotide
cgtcccntgg

SEQ ID NO: 220 - DNA, 10 bp, Artificial
  Oligonucleotide
cgtccccngg

SEQ ID NO: 221 - DNA, 10 bp, Artificial
  Oligonucleotide
cgtcccccgg

SEQ ID NO: 222 - DNA, 10 bp, Artificial
  Oligonucleotide
cgtccccggg

SEQ ID NO: 223 - DNA, 10 bp, Artificial
  Oligonucleotide
cgtcccctgg

SEQ ID NO: 224 - DNA, 10 bp, Artificial
  Oligonucleotide
cgtcccgagg

SEQ ID NO: 225 - DNA, 10 bp, Artificial
  Oligonucleotide
cgtcccgcgg

SEQ ID NO: 226 - DNA, 10 bp, Artificial
  Oligonucleotide
cgtcccgggg

SEQ ID NO: 227 - DNA, 10 bp, Artificial
  Oligonucleotide
cgtcccgtgg

SEQ ID NO: 228 - DNA, 10 bp, Artificial
  Oligonucleotide
cgtccctngg

SEQ ID NO: 229 - DNA, 10 bp, Artificial
  Oligonucleotide
cgtccctcgg

SEQ ID NO: 230 - DNA, 10 bp, Artificial
  Oligonucleotide
cgtccctggg

SEQ ID NO: 231 - DNA, 10 bp, Artificial
  Oligonucleotide
cgtcccttgg

SEQ ID NO: 232 - DNA, 10 bp, Artificial
  Oligonucleotide
cgtccgnngg

SEQ ID NO: 233 - DNA, 10 bp, Artificial
  Oligonucleotide
cgtccgncgg

SEQ ID NO: 234 - DNA, 10 bp, Artificial
  Oligonucleotide
cgtccgnggg

SEQ ID NO: 235 - DNA, 10 bp, Artificial
  Oligonucleotide
cgtccgntgg

SEQ ID NO: 236 - DNA, 10 bp, Artificial
  Oligonucleotide
cgtccgcngg

SEQ ID NO: 237 - DNA, 10 bp, Artificial
  Oligonucleotide
cgtccgccgg

SEQ ID NO: 238 - DNA, 10 bp, Artificial
  Oligonucleotide
cgtccgcggg

SEQ ID NO: 239 - DNA, 10 bp, Artificial
  Oligonucleotide
cgtccgctgg

SEQ ID NO: 240 - DNA, 10 bp, Artificial
  Oligonucleotide
cgtccggngg

SEQ ID NO: 241 - DNA, 10 bp, Artificial
  Oligonucleotide
cgtccggcgg

SEQ ID NO: 242 - DNA, 10 bp, Artificial
  Oligonucleotide
cgtccggggg

SEQ ID NO: 243 - DNA, 10 bp, Artificial
  Oligonucleotide
cgtccggtgg

SEQ ID NO: 244 - DNA, 10 bp, Artificial
  Oligonucleotide
cgtccgtngg

SEQ ID NO: 245 - DNA, 10 bp, Artificial
  Oligonucleotide
cgtccgtcgg

SEQ ID NO: 246 - DNA, 10 bp, Artificial
  Oligonucleotide
cgtccgtggg

SEQ ID NO: 247 - DNA, 10 bp, Artificial
  Oligonucleotide
cgtccgttgg

SEQ ID NO: 248 - DNA, 10 bp, Artificial
  Oligonucleotide
cgtcctnngg

SEQ ID NO: 249 - DNA, 10 bp, Artificial
  Oligonucleotide
cgtcctncgg

SEQ ID NO: 250 - DNA, 10 bp, Artificial
  Oligonucleotide
cgtcctnggg

SEQ ID NO: 251 - DNA, 10 bp, Artificial
  Oligonucleotide
cgtcctntgg

SEQ ID NO: 252 - DNA, 10 bp, Artificial
  Oligonucleotide
cgtcctcngg

SEQ ID NO: 253 - DNA, 10 bp, Artificial
  Oligonucleotide
cgtcctccgg

SEQ ID NO: 254 - DNA, 10 bp, Artificial
  Oligonucleotide
cgtcctcggg

SEQ ID NO: 255 - DNA, 10 bp, Artificial
  Oligonucleotide
cgtcctctgg

SEQ ID NO: 256 - DNA, 10 bp, Artificial
  Oligonucleotide
cgtcctgngg

SEQ ID NO: 257 - DNA, 10 bp, Artificial
  Oligonucleotide
cgtcctgcgg

SEQ ID NO: 258 - DNA, 10 bp, Artificial
  Oligonucleotide
cgtcctgggg

SEQ ID NO: 259 - DNA, 10 bp, Artificial
  Oligonucleotide
cgtcctgtgg

SEQ ID NO: 260 - DNA, 10 bp, Artificial
  Oligonucleotide
cgtccttngg

SEQ ID NO: 261 - DNA, 10 bp, Artificial
  Oligonucleotide
cgtccttcgg

SEQ ID NO: 262 - DNA, 10 bp, Artificial
  Oligonucleotide
cgtccttggg

SEQ ID NO: 263 - DNA, 10 bp, Artificial
  Oligonucleotide
cgtcctttgg

SEQ ID NO: 264 - DNA, 15 bp, Artificial
  Oligonucleotide
caggcssnnnggacg

SEQ ID NO: 265 - DNA, 15 bp, Artificial
  Oligonucleotide
ctgccssnnnggacg

SEQ ID NO: 266 - DNA, 15 bp, Artificial
  Oligonucleotide
cagccssnnnggacg

SEQ ID NO: 267 - DNA, 15 bp, Artificial
  Oligonucleotide
gcgttssnnnggacg

SEQ ID NO: 268 - DNA, 15 bp, Artificial
  Oligonucleotide
aacgcssnnnggacg

SEQ ID NO: 269 - DNA, 15 bp, Artificial
  Oligonucleotide
acggtssnnnggacg

SEQ ID NO: 270 - DNA, 15 bp, Artificial
  Oligonucleotide
accgtssnnnggacg

SEQ ID NO: 271 - DNA, 15 bp, Artificial
  Oligonucleotide
gtcggssnnnggacg

SEQ ID NO: 272 - DNA, 15 bp, Artificial
  Oligonucleotide
gacggssnnnggacg

SEQ ID NO: 273 - DNA, 15 bp, Artificial
  Oligonucleotide
ggtcgssnnnggacg

SEQ ID NO: 274 - DNA, 15 bp, Artificial
  Oligonucleotide
gtccgssnnnggacg

SEQ ID NO: 275 - DNA, 15 bp, Artificial
  Oligonucleotide
gaccgssnnnggacg

SEQ ID NO: 276 - DNA, 15 bp, Artificial
  Oligonucleotide
ggacgssnnnggacg

SEQ ID NO: 277 - DNA, 15 bp, Artificial
  Oligonucleotide
cggtcssnnnggacg

SEQ ID NO: 278 - DNA, 15 bp, Artificial
  Oligonucleotide
ccgtcssnnnggacg

SEQ ID NO: 279 - DNA, 15 bp, Artificial
  Oligonucleotide
cgtccssnnnggacg

SEQ ID NO: 280 - DNA, 15 bp, Artificial
  Oligonucleotide
cgaccssnnnggacg

SEQ ID NO: 281 - DNA, 15 bp, Artificial
  Oligonucleotide
cggacssnnnggacg

SEQ ID NO: 282 - DNA, 15 bp, Artificial
  Oligonucleotide
aggcassnnnggacg

SEQ ID NO: 283 - DNA, 15 bp, Artificial
  Oligonucleotide
tgcctssnnnggacg

SEQ ID NO: 284 - DNA, 15 bp, Artificial
  Oligonucleotide
cgcatssnnnggacg

SEQ ID NO: 285 - DNA, 15 bp, Artificial
  Oligonucleotide
atgcgssnnnggacg

SEQ ID NO: 286 - DNA, 15 bp, Artificial
  Oligonucleotide
gcctcssnnnggacg

SEQ ID NO: 287 - DNA, 15 bp, Artificial
  Oligonucleotide
gaggcssnnnggacg

SEQ ID NO: 288 - DNA, 15 bp, Artificial
  Oligonucleotide
ttcgcssnnnggacg

SEQ ID NO: 289 - DNA, 15 bp, Artificial
  Oligonucleotide
gctccssnnnggacg

SEQ ID NO: 290 - DNA, 15 bp, Artificial
  Oligonucleotide
gagccssnnnggacg

SEQ ID NO: 291 - DNA, 15 bp, Artificial
  Oligonucleotide
gcgaassnnnggacg

SEQ ID NO: 292 - DNA, 15 bp, Artificial
  Oligonucleotide
tcggtssnnnggacg

SEQ ID NO: 293 - DNA, 15 bp, Artificial
  Oligonucleotide
tccgtssnnnggacg

SEQ ID NO: 294 - DNA, 15 bp, Artificial
  Oligonucleotide
acggassnnnggacg

SEQ ID NO: 295 - DNA, 15 bp, Artificial
  Oligonucleotide
accgassnnnggacg

SEQ ID NO: 296 - DNA, 15 bp, Artificial
  Oligonucleotide
cgcttssnnnggacg

SEQ ID NO: 297 - DNA, 15 bp, Artificial
  Oligonucleotide
aagcgssnnnggacg

SEQ ID NO: 298 - DNA, 15 bp, Artificial
  Oligonucleotide
ctcggssnnnggacg

SEQ ID NO: 299 - DNA, 15 bp, Artificial
  Oligonucleotide
cgaggssnnnggacg

SEQ ID NO: 300 - DNA, 15 bp, Artificial
  Oligonucleotide
cctcgssnnnggacg

SEQ ID NO: 301 - DNA, 15 bp, Artificial
  Oligonucleotide
ctccgssnnnggacg

SEQ ID NO: 302 - DNA, 15 bp, Artificial
  Oligonucleotide
cggagssnnnggacg

SEQ ID NO: 303 - DNA, 15 bp, Artificial
  Oligonucleotide
ccgagssnnnggacg

SEQ ID NO: 304 - DNA, 15 bp, Artificial
  Oligonucleotide
ccaccssnnnggacg

SEQ ID NO: 305 - DNA, 15 bp, Artificial
  Oligonucleotide
gggtgssnnnggacg

SEQ ID NO: 306 - DNA, 15 bp, Artificial
  Oligonucleotide
ggtggssnnnggacg

SEQ ID NO: 307 - DNA, 15 bp, Artificial
  Oligonucleotide
gtgggssnnnggacg

SEQ ID NO: 308 - DNA, 15 bp, Artificial
  Oligonucleotide
cacccssnnnggacg

SEQ ID NO: 309 - DNA, 15 bp, Artificial
  Oligonucleotide
cccacssnnnggacg

SEQ ID NO: 310 - DNA, 15 bp, Artificial
  Oligonucleotide
gcgatssnnnggacg

SEQ ID NO: 311 - DNA, 15 bp, Artificial
  Oligonucleotide
atcgcssnnnggacg

SEQ ID NO: 312 - DNA, 15 bp, Artificial
  Oligonucleotide
agcctssnnnggacg

SEQ ID NO: 313 - DNA, 15 bp, Artificial
  Oligonucleotide
tgtgcssnnnggacg

SEQ ID NO: 314 - DNA, 15 bp, Artificial
  Oligonucleotide
tgcacssnnnggacg

SEQ ID NO: 315 - DNA, 15 bp, Artificial
  Oligonucleotide
gtgcassnnnggacg

SEQ ID NO: 316 - DNA, 15 bp, Artificial
  Oligonucleotide
gcacassnnnggacg

SEQ ID NO: 317 - DNA, 15 bp, Artificial
  Oligonucleotide
tcggassnnnggacg

SEQ ID NO: 318 - DNA, 15 bp, Artificial
  Oligonucleotide
tccgassnnnggacg

SEQ ID NO: 319 - DNA, 15 bp, Artificial
  Oligonucleotide
ttgccssnnnggacg

SEQ ID NO: 320 - DNA, 15 bp, Artificial
  Oligonucleotide
gccaassnnnggacg

SEQ ID NO: 321 - DNA, 15 bp, Artificial
  Oligonucleotide
tgggtssnnnggacg

SEQ ID NO: 322 - DNA, 15 bp, Artificial
  Oligonucleotide
acccassnnnggacg

SEQ ID NO: 323 - DNA, 15 bp, Artificial
  Oligonucleotide
cgtgtssnnnggacg

SEQ ID NO: 324 - DNA, 15 bp, Artificial
  Oligonucleotide
cacgtssnnnggacg

SEQ ID NO: 325 - DNA, 15 bp, Artificial
  Oligonucleotide
acgtgssnnnggacg

SEQ ID NO: 326 - DNA, 15 bp, Artificial
  Oligonucleotide
acacgssnnnggacg

SEQ ID NO: 327 - DNA, 15 bp, Artificial
  Oligonucleotide
gggtcssnnnggacg

SEQ ID NO: 328 - DNA, 15 bp, Artificial
  Oligonucleotide
ggtccssnnnggacg

SEQ ID NO: 329 - DNA, 15 bp, Artificial
  Oligonucleotide
gtcccssnnnggacg

SEQ ID NO: 330 - DNA, 15 bp, Artificial
  Oligonucleotide
gacccssnnnggacg

SEQ ID NO: 331 - DNA, 15 bp, Artificial
  Oligonucleotide
ggaccssnnnggacg

SEQ ID NO: 332 - DNA, 15 bp, Artificial
  Oligonucleotide
gggacssnnnggacg

SEQ ID NO: 333 - DNA, 15 bp, Artificial
  Oligonucleotide
ccctgssnnnggacg

SEQ ID NO: 334 - DNA, 15 bp, Artificial
  Oligonucleotide
ctgggssnnnggacg

SEQ ID NO: 335 - DNA, 15 bp, Artificial
  Oligonucleotide
cagggssnnnggacg

SEQ ID NO: 336 - DNA, 15 bp, Artificial
  Oligonucleotide
ccaggssnnnggacg

SEQ ID NO: 337 - DNA, 15 bp, Artificial
  Oligonucleotide
cccagssnnnggacg

SEQ ID NO: 338 - DNA, 15 bp, Artificial
  Oligonucleotide
cggttssnnnggacg

SEQ ID NO: 339 - DNA, 15 bp, Artificial
  Oligonucleotide
ccgttssnnnggacg

SEQ ID NO: 340 - DNA, 15 bp, Artificial
  Oligonucleotide
aacggssnnnggacg

SEQ ID NO: 341 - DNA, 15 bp, Artificial
  Oligonucleotide
aaccgssnnnggacg

SEQ ID NO: 342 - DNA, 15 bp, Artificial
  Oligonucleotide
tacgcssnnnggacg

SEQ ID NO: 343 - DNA, 15 bp, Artificial
  Oligonucleotide
gcgtassnnnggacg

SEQ ID NO: 344 - DNA, 15 bp, Artificial
  Oligonucleotide
tgctgssnnnggacg

SEQ ID NO: 345 - DNA, 15 bp, Artificial
  Oligonucleotide
tgcagssnnnggacg

SEQ ID NO: 346 - DNA, 15 bp, Artificial
  Oligonucleotide
ctgcassnnnggacg

SEQ ID NO: 347 - DNA, 15 bp, Artificial
  Oligonucleotide
cagcassnnnggacg

SEQ ID NO: 348 - DNA, 15 bp, Artificial
  Oligonucleotide
gctgtssnnnggacg

SEQ ID NO: 349 - DNA, 15 bp, Artificial
  Oligonucleotide
gcagtssnnnggacg

SEQ ID NO: 350 - DNA, 15 bp, Artificial
  Oligonucleotide
gtgctssnnnggacg

SEQ ID NO: 351 - DNA, 15 bp, Artificial
  Oligonucleotide
gcactssnnnggacg

SEQ ID NO: 352 - DNA, 15 bp, Artificial
  Oligonucleotide
gccatssnnnggacg

SEQ ID NO: 353 - DNA, 15 bp, Artificial
  Oligonucleotide
agtgcssnnnggacg

SEQ ID NO: 354 - DNA, 15 bp, Artificial
  Oligonucleotide
actgcssnnnggacg

SEQ ID NO: 355 - DNA, 15 bp, Artificial
  Oligonucleotide
atggcssnnnggacg

SEQ ID NO: 356 - DNA, 15 bp, Artificial
  Oligonucleotide
acagcssnnnggacg

SEQ ID NO: 357 - DNA, 15 bp, Artificial
  Oligonucleotide
atgccssnnnggacg

SEQ ID NO: 358 - DNA, 15 bp, Artificial
  Oligonucleotide
agcacssnnnggacg

SEQ ID NO: 359 - DNA, 15 bp, Artificial
  Oligonucleotide
tgggassnnnggacg

SEQ ID NO: 360 - DNA, 15 bp, Artificial
  Oligonucleotide
tcccassnnnggacg

SEQ ID NO: 361 - DNA, 15 bp, Artificial
  Oligonucleotide
tcgtgssnnnggacg

SEQ ID NO: 362 - DNA, 15 bp, Artificial
  Oligonucleotide
tcacgssnnnggacg

SEQ ID NO: 363 - DNA, 15 bp, Artificial
  Oligonucleotide
cgtgassnnnggacg

SEQ ID NO: 364 - DNA, 15 bp, Artificial
  Oligonucleotide
cgtcassnnnggacg

SEQ ID NO: 365 - DNA, 15 bp, Artificial
  Oligonucleotide
cgacassnnnggacg

SEQ ID NO: 366 - DNA, 15 bp, Artificial
  Oligonucleotide
gccttssnnnggacg

SEQ ID NO: 367 - DNA, 15 bp, Artificial
  Oligonucleotide
tgtcgssnnnggacg

SEQ ID NO: 368 - DNA, 15 bp, Artificial
  Oligonucleotide
tgacgssnnnggacg

SEQ ID NO: 369 - DNA, 15 bp, Artificial
  Oligonucleotide
aaggcssnnnggacg

SEQ ID NO: 370 - DNA, 15 bp, Artificial
  Oligonucleotide
aagccssnnnggacg

SEQ ID NO: 371 - DNA, 15 bp, Artificial
  Oligonucleotide
cacgassnnnggacg

SEQ ID NO: 372 - DNA, 15 bp, Artificial
  Oligonucleotide
agggtssnnnggacg

SEQ ID NO: 373 - DNA, 15 bp, Artificial
  Oligonucleotide
gtcgtssnnnggacg

SEQ ID NO: 374 - DNA, 15 bp, Artificial
  Oligonucleotide
gacgtssnnnggacg

SEQ ID NO: 375 - DNA, 15 bp, Artificial
  Oligonucleotide
accctssnnnggacg

SEQ ID NO: 376 - DNA, 15 bp, Artificial
  Oligonucleotide
acgtcssnnnggacg

SEQ ID NO: 377 - DNA, 15 bp, Artificial
  Oligonucleotide
acgacssnnnggacg

SEQ ID NO: 378 - DNA, 15 bp, Artificial
  Oligonucleotide
gagggssnnnggacg

SEQ ID NO: 379 - DNA, 15 bp, Artificial
  Oligonucleotide
ttcggssnnnggacg

SEQ ID NO: 380 - DNA, 15 bp, Artificial
  Oligonucleotide
ggaggssnnnggacg

SEQ ID NO: 381 - DNA, 15 bp, Artificial
  Oligonucleotide
ttccgssnnnggacg

SEQ ID NO: 382 - DNA, 15 bp, Artificial
  Oligonucleotide
gggagssnnnggacg

SEQ ID NO: 383 - DNA, 15 bp, Artificial
  Oligonucleotide
cggaassnnnggacg

SEQ ID NO: 384 - DNA, 15 bp, Artificial
  Oligonucleotide
ccgaassnnnggacg

SEQ ID NO: 385 - DNA, 15 bp, Artificial
  Oligonucleotide
tctgcssnnnggacg

SEQ ID NO: 386 - DNA, 15 bp, Artificial
  Oligonucleotide
tcagcssnnnggacg

SEQ ID NO: 387 - DNA, 15 bp, Artificial
  Oligonucleotide
cgctassnnnggacg

SEQ ID NO: 388 - DNA, 15 bp, Artificial
  Oligonucleotide
gagcassnnnggacg

SEQ ID NO: 389 - DNA, 15 bp, Artificial
  Oligonucleotide
tagcgssnnnggacg

SEQ ID NO: 390 - DNA, 15 bp, Artificial
  Oligonucleotide
tgctcssnnnggacg

SEQ ID NO: 391 - DNA, 15 bp, Artificial
  Oligonucleotide
tgagcssnnnggacg

SEQ ID NO: 392 - DNA, 15 bp, Artificial
  Oligonucleotide
gctgassnnnggacg

SEQ ID NO: 393 - DNA, 15 bp, Artificial
  Oligonucleotide
gcagassnnnggacg

SEQ ID NO: 394 - DNA, 15 bp, Artificial
  Oligonucleotide
gctcassnnnggacg

SEQ ID NO: 395 - DNA, 15 bp, Artificial
  Oligonucleotide
ctgctssnnnggacg

SEQ ID NO: 396 - DNA, 15 bp, Artificial
  Oligonucleotide
cagctssnnnggacg

SEQ ID NO: 397 - DNA, 15 bp, Artificial
  Oligonucleotide
agctgssnnnggacg

SEQ ID NO: 398 - DNA, 15 bp, Artificial
  Oligonucleotide
agcagssnnnggacg

SEQ ID NO: 399 - DNA, 15 bp, Artificial
  Oligonucleotide
tccctssnnnggacg

SEQ ID NO: 400 - DNA, 15 bp, Artificial
  Oligonucleotide
tcgtcssnnnggacg

SEQ ID NO: 401 - DNA, 15 bp, Artificial
  Oligonucleotide
tcgacssnnnggacg

SEQ ID NO: 402 - DNA, 15 bp, Artificial
  Oligonucleotide
agggassnnnggacg

SEQ ID NO: 403 - DNA, 15 bp, Artificial
  Oligonucleotide
gtcgassnnnggacg

SEQ ID NO: 404 - DNA, 15 bp, Artificial
  Oligonucleotide
gacgassnnnggacg

SEQ ID NO: 405 - DNA, 15 bp, Artificial
  Oligonucleotide
cgagtssnnnggacg

SEQ ID NO: 406 - DNA, 15 bp, Artificial
  Oligonucleotide
cgtctssnnnggacg

SEQ ID NO: 407 - DNA, 15 bp, Artificial
  Oligonucleotide
cgactssnnnggacg

SEQ ID NO: 408 - DNA, 15 bp, Artificial
  Oligonucleotide
cggatssnnnggacg

SEQ ID NO: 409 - DNA, 15 bp, Artificial
  Oligonucleotide
ccgatssnnnggacg

SEQ ID NO: 410 - DNA, 15 bp, Artificial
  Oligonucleotide
atcggssnnnggacg

SEQ ID NO: 411 - DNA, 15 bp, Artificial
  Oligonucleotide
actcgssnnnggacg

SEQ ID NO: 412 - DNA, 15 bp, Artificial
  Oligonucleotide
atccgssnnnggacg

SEQ ID NO: 413 - DNA, 15 bp, Artificial
  Oligonucleotide
acgagssnnnggacg

SEQ ID NO: 414 - DNA, 15 bp, Artificial
  Oligonucleotide
ctcgtssnnnggacg

SEQ ID NO: 415 - DNA, 15 bp, Artificial
  Oligonucleotide
agtcgssnnnggacg

SEQ ID NO: 416 - DNA, 15 bp, Artificial
  Oligonucleotide
agacgssnnnggacg

SEQ ID NO: 417 - DNA, 15 bp, Artificial
  Oligonucleotide
tggtgssnnnggacg

SEQ ID NO: 418 - DNA, 15 bp, Artificial
  Oligonucleotide
tgtggssnnnggacg

SEQ ID NO: 419 - DNA, 15 bp, Artificial
  Oligonucleotide
gttgcssnnnggacg

SEQ ID NO: 420 - DNA, 15 bp, Artificial
  Oligonucleotide
gcaacssnnnggacg

SEQ ID NO: 421 - DNA, 15 bp, Artificial
  Oligonucleotide
caccassnnnggacg

SEQ ID NO: 422 - DNA, 15 bp, Artificial
  Oligonucleotide
ccacassnnnggacg

SEQ ID NO: 423 - DNA, 15 bp, Artificial
  Oligonucleotide
ggtgtssnnnggacg

SEQ ID NO: 424 - DNA, 15 bp, Artificial
  Oligonucleotide
gtggtssnnnggacg

SEQ ID NO: 425 - DNA, 15 bp, Artificial
  Oligonucleotide
acaccssnnnggacg

SEQ ID NO: 426 - DNA, 15 bp, Artificial
  Oligonucleotide
accacssnnnggacg

SEQ ID NO: 427 - DNA, 15 bp, Artificial
  Oligonucleotide
ttgggssnnnggacg

SEQ ID NO: 428 - DNA, 15 bp, Artificial
  Oligonucleotide
cccaassnnnggacg

SEQ ID NO: 429 - DNA, 15 bp, Artificial
  Oligonucleotide
gggttssnnnggacg

SEQ ID NO: 430 - DNA, 15 bp, Artificial
  Oligonucleotide
aacccssnnnggacg

SEQ ID NO: 431 - DNA, 15 bp, Artificial
  Oligonucleotide
cgttgssnnnggacg

SEQ ID NO: 432 - DNA, 15 bp, Artificial
  Oligonucleotide
caacgssnnnggacg

SEQ ID NO: 433 - DNA, 15 bp, Artificial
  Oligonucleotide
gctctssnnnggacg

SEQ ID NO: 434 - DNA, 15 bp, Artificial
  Oligonucleotide
gagctssnnnggacg

SEQ ID NO: 435 - DNA, 15 bp, Artificial
  Oligonucleotide
agctcssnnnggacg

SEQ ID NO: 436 - DNA, 15 bp, Artificial
  Oligonucleotide
agagcssnnnggacg

SEQ ID NO: 437 - DNA, 15 bp, Artificial
  Oligonucleotide
ttgcassnnnggacg

SEQ ID NO: 438 - DNA, 15 bp, Artificial
  Oligonucleotide
tgcaassnnnggacg

SEQ ID NO: 439 - DNA, 15 bp, Artificial
  Oligonucleotide
gcatgssnnnggacg

SEQ ID NO: 440 - DNA, 15 bp, Artificial
  Oligonucleotide
catgcssnnnggacg

SEQ ID NO: 441 - DNA, 15 bp, Artificial
  Oligonucleotide
tctcgssnnnggacg

SEQ ID NO: 442 - DNA, 15 bp, Artificial
  Oligonucleotide
tcgagssnnnggacg

SEQ ID NO: 443 - DNA, 15 bp, Artificial
  Oligonucleotide
ctcgassnnnggacg

SEQ ID NO: 444 - DNA, 15 bp, Artificial
  Oligonucleotide
cgagassnnnggacg

SEQ ID NO: 445 - DNA, 15 bp, Artificial
  Oligonucleotide
tacggssnnnggacg

SEQ ID NO: 446 - DNA, 15 bp, Artificial
  Oligonucleotide
taccgssnnnggacg

SEQ ID NO: 447 - DNA, 15 bp, Artificial
  Oligonucleotide
tggtcssnnnggacg

SEQ ID NO: 448 - DNA, 15 bp, Artificial
  Oligonucleotide
tgtccssnnnggacg

SEQ ID NO: 449 - DNA, 15 bp, Artificial
  Oligonucleotide
tgaccssnnnggacg

SEQ ID NO: 450 - DNA, 15 bp, Artificial
  Oligonucleotide
tcaccssnnnggacg

SEQ ID NO: 451 - DNA, 15 bp, Artificial
  Oligonucleotide
tggacssnnnggacg

SEQ ID NO: 452 - DNA, 15 bp, Artificial
  Oligonucleotide
tccacssnnnggacg

SEQ ID NO: 453 - DNA, 15 bp, Artificial
  Oligonucleotide
cggtassnnnggacg

SEQ ID NO: 454 - DNA, 15 bp, Artificial
  Oligonucleotide
ccgtassnnnggacg

SEQ ID NO: 455 - DNA, 15 bp, Artificial
  Oligonucleotide
ggtgassnnnggacg

SEQ ID NO: 456 - DNA, 15 bp, Artificial
  Oligonucleotide
gtggassnnnggacg

SEQ ID NO: 457 - DNA, 15 bp, Artificial
  Oligonucleotide
ggtcassnnnggacg

SEQ ID NO: 458 - DNA, 15 bp, Artificial
  Oligonucleotide
gtccassnnnggacg

SEQ ID NO: 459 - DNA, 15 bp, Artificial
  Oligonucleotide
gaccassnnnggacg

SEQ ID NO: 460 - DNA, 15 bp, Artificial
  Oligonucleotide
ggacassnnnggacg

SEQ ID NO: 461 - DNA, 15 bp, Artificial
  Oligonucleotide
gcttgssnnnggacg

SEQ ID NO: 462 - DNA, 15 bp, Artificial
  Oligonucleotide
gcaagssnnnggacg

SEQ ID NO: 463 - DNA, 15 bp, Artificial
  Oligonucleotide
cttgcssnnnggacg

SEQ ID NO: 464 - DNA, 15 bp, Artificial
  Oligonucleotide
caagcssnnnggacg

SEQ ID NO: 465 - DNA, 15 bp, Artificial
  Oligonucleotide
cctgtssnnnggacg

SEQ ID NO: 466 - DNA, 15 bp, Artificial
  Oligonucleotide
ctggtssnnnggacg

SEQ ID NO: 467 - DNA, 15 bp, Artificial
  Oligonucleotide
caggtssnnnggacg

SEQ ID NO: 468 - DNA, 15 bp, Artificial
  Oligonucleotide
ccagtssnnnggacg

SEQ ID NO: 469 - DNA, 15 bp, Artificial
  Oligonucleotide
cacctssnnnggacg

SEQ ID NO: 470 - DNA, 15 bp, Artificial
  Oligonucleotide
ccactssnnnggacg

SEQ ID NO: 471 - DNA, 15 bp, Artificial
  Oligonucleotide
cccatssnnnggacg

SEQ ID NO: 472 - DNA, 15 bp, Artificial
  Oligonucleotide
aggtgssnnnggacg

SEQ ID NO: 473 - DNA, 15 bp, Artificial
  Oligonucleotide
acctgssnnnggacg

SEQ ID NO: 474 - DNA, 15 bp, Artificial
  Oligonucleotide
accagssnnnggacg

SEQ ID NO: 475 - DNA, 15 bp, Artificial
  Oligonucleotide
acgttssnnnggacg

SEQ ID NO: 476 - DNA, 15 bp, Artificial
  Oligonucleotide
aacgtssnnnggacg

SEQ ID NO: 477 - DNA, 15 bp, Artificial
  Oligonucleotide
ttcccssnnnggacg

SEQ ID NO: 478 - DNA, 15 bp, Artificial
  Oligonucleotide
gggaassnnnggacg

SEQ ID NO: 479 - DNA, 15 bp, Artificial
  Oligonucleotide
atgcassnnnggacg

SEQ ID NO: 480 - DNA, 15 bp, Artificial
  Oligonucleotide
tgcatssnnnggacg

SEQ ID NO: 481 - DNA, 15 bp, Artificial
  Oligonucleotide
cccttssnnnggacg

SEQ ID NO: 482 - DNA, 15 bp, Artificial
  Oligonucleotide
aagggssnnnggacg

SEQ ID NO: 483 - DNA, 15 bp, Artificial
  Oligonucleotide
gttcgssnnnggacg

SEQ ID NO: 484 - DNA, 15 bp, Artificial
  Oligonucleotide
gaacgssnnnggacg

SEQ ID NO: 485 - DNA, 15 bp, Artificial
  Oligonucleotide
cgttcssnnnggacg

SEQ ID NO: 486 - DNA, 15 bp, Artificial
  Oligonucleotide
cgaacssnnnggacg

SEQ ID NO: 487 - DNA, 15 bp, Artificial
  Oligonucleotide
taggcssnnnggacg

SEQ ID NO: 488 - DNA, 15 bp, Artificial
  Oligonucleotide
tagccssnnnggacg

SEQ ID NO: 489 - DNA, 15 bp, Artificial
  Oligonucleotide
tgcttssnnnggacg

SEQ ID NO: 490 - DNA, 15 bp, Artificial
  Oligonucleotide
ttgctssnnnggacg

SEQ ID NO: 491 - DNA, 15 bp, Artificial
  Oligonucleotide
gcatcssnnnggacg

SEQ ID NO: 492 - DNA, 15 bp, Artificial
  Oligonucleotide
gatgcssnnnggacg

SEQ ID NO: 493 - DNA, 15 bp, Artificial
  Oligonucleotide
aagcassnnnggacg

SEQ ID NO: 494 - DNA, 15 bp, Artificial
  Oligonucleotide
agcaassnnnggacg

SEQ ID NO: 495 - DNA, 15 bp, Artificial
  Oligonucleotide
tcctgssnnnggacg

SEQ ID NO: 496 - DNA, 15 bp, Artificial
  Oligonucleotide
tctggssnnnggacg

SEQ ID NO: 497 - DNA, 15 bp, Artificial
  Oligonucleotide
tgaggssnnnggacg

SEQ ID NO: 498 - DNA, 15 bp, Artificial
  Oligonucleotide
tcaggssnnnggacg

SEQ ID NO: 499 - DNA, 15 bp, Artificial
  Oligonucleotide
tggagssnnnggacg

SEQ ID NO: 500 - DNA, 15 bp, Artificial
  Oligonucleotide
tccagssnnnggacg

SEQ ID NO: 501 - DNA, 15 bp, Artificial
  Oligonucleotide
gcttcssnnnggacg

SEQ ID NO: 502 - DNA, 15 bp, Artificial
  Oligonucleotide
gaagcssnnnggacg

SEQ ID NO: 503 - DNA, 15 bp, Artificial
  Oligonucleotide
cctgassnnnggacg

SEQ ID NO: 504 - DNA, 15 bp, Artificial
  Oligonucleotide
ctggassnnnggacg

SEQ ID NO: 505 - DNA, 15 bp, Artificial
  Oligonucleotide
caggassnnnggacg

SEQ ID NO: 506 - DNA, 15 bp, Artificial
  Oligonucleotide
ccagassnnnggacg

SEQ ID NO: 507 - DNA, 15 bp, Artificial
  Oligonucleotide
cctcassnnnggacg

SEQ ID NO: 508 - DNA, 15 bp, Artificial
  Oligonucleotide
ctccassnnnggacg

SEQ ID NO: 509 - DNA, 15 bp, Artificial
  Oligonucleotide
tcgttssnnnggacg

SEQ ID NO: 510 - DNA, 15 bp, Artificial
  Oligonucleotide
gaggtssnnnggacg

SEQ ID NO: 511 - DNA, 15 bp, Artificial
  Oligonucleotide
ttcgtssnnnggacg

SEQ ID NO: 512 - DNA, 15 bp, Artificial
  Oligonucleotide
ggagtssnnnggacg

SEQ ID NO: 513 - DNA, 15 bp, Artificial
  Oligonucleotide
ggtctssnnnggacg

SEQ ID NO: 514 - DNA, 15 bp, Artificial
  Oligonucleotide
gtcctssnnnggacg

SEQ ID NO: 515 - DNA, 15 bp, Artificial
  Oligonucleotide
ggactssnnnggacg

SEQ ID NO: 516 - DNA, 15 bp, Artificial
  Oligonucleotide
gggatssnnnggacg

SEQ ID NO: 517 - DNA, 15 bp, Artificial
  Oligonucleotide
aggtcssnnnggacg

SEQ ID NO: 518 - DNA, 15 bp, Artificial
  Oligonucleotide
acctcssnnnggacg

SEQ ID NO: 519 - DNA, 15 bp, Artificial
  Oligonucleotide
agtccssnnnggacg

SEQ ID NO: 520 - DNA, 15 bp, Artificial
  Oligonucleotide
actccssnnnggacg

SEQ ID NO: 521 - DNA, 15 bp, Artificial
  Oligonucleotide
atcccssnnnggacg

SEQ ID NO: 522 - DNA, 15 bp, Artificial
  Oligonucleotide
agaccssnnnggacg

SEQ ID NO: 523 - DNA, 15 bp, Artificial
  Oligonucleotide
aggacssnnnggacg

SEQ ID NO: 524 - DNA, 15 bp, Artificial
  Oligonucleotide
aacgassnnnggacg

SEQ ID NO: 525 - DNA, 15 bp, Artificial
  Oligonucleotide
acgaassnnnggacg

SEQ ID NO: 526 - DNA, 15 bp, Artificial
  Oligonucleotide
gacctssnnnggacg

SEQ ID NO: 527 - DNA, 15 bp, Artificial
  Oligonucleotide
cgatgssnnnggacg

SEQ ID NO: 528 - DNA, 15 bp, Artificial
  Oligonucleotide
catcgssnnnggacg

SEQ ID NO: 529 - DNA, 15 bp, Artificial
  Oligonucleotide
gtgtgssnnnggacg

SEQ ID NO: 530 - DNA, 15 bp, Artificial
  Oligonucleotide
cacacssnnnggacg

SEQ ID NO: 531 - DNA, 15 bp, Artificial
  Oligonucleotide
atgctssnnnggacg

SEQ ID NO: 532 - DNA, 15 bp, Artificial
  Oligonucleotide
cttcgssnnnggacg

SEQ ID NO: 533 - DNA, 15 bp, Artificial
  Oligonucleotide
cgaagssnnnggacg

SEQ ID NO: 534 - DNA, 15 bp, Artificial
  Oligonucleotide
agcatssnnnggacg

SEQ ID NO: 535 - DNA, 15 bp, Artificial
  Oligonucleotide
ggttgssnnnggacg

SEQ ID NO: 536 - DNA, 15 bp, Artificial
  Oligonucleotide
gttggssnnnggacg

SEQ ID NO: 537 - DNA, 15 bp, Artificial
  Oligonucleotide
agcttssnnnggacg

SEQ ID NO: 538 - DNA, 15 bp, Artificial
  Oligonucleotide
atcgtssnnnggacg

SEQ ID NO: 539 - DNA, 15 bp, Artificial
  Oligonucleotide
acgatssnnnggacg

SEQ ID NO: 540 - DNA, 15 bp, Artificial
  Oligonucleotide
tgtgtssnnnggacg

SEQ ID NO: 541 - DNA, 15 bp, Artificial
  Oligonucleotide
acacassnnnggacg

SEQ ID NO: 542 - DNA, 15 bp, Artificial
  Oligonucleotide
gaggassnnnggacg

SEQ ID NO: 543 - DNA, 15 bp, Artificial
  Oligonucleotide
ttcgassnnnggacg

SEQ ID NO: 544 - DNA, 15 bp, Artificial
  Oligonucleotide
cctctssnnnggacg

SEQ ID NO: 545 - DNA, 15 bp, Artificial
  Oligonucleotide
ctcctssnnnggacg

SEQ ID NO: 546 - DNA, 15 bp, Artificial
  Oligonucleotide
agaggssnnnggacg

SEQ ID NO: 547 - DNA, 15 bp, Artificial
  Oligonucleotide
gatcgssnnnggacg

SEQ ID NO: 548 - DNA, 15 bp, Artificial
  Oligonucleotide
aggagssnnnggacg

SEQ ID NO: 549 - DNA, 15 bp, Artificial
  Oligonucleotide
cgatcssnnnggacg

SEQ ID NO: 550 - DNA, 15 bp, Artificial
  Oligonucleotide
tacccssnnnggacg

SEQ ID NO: 551 - DNA, 15 bp, Artificial
  Oligonucleotide
gggtassnnnggacg

SEQ ID NO: 552 - DNA, 15 bp, Artificial
  Oligonucleotide
tttgcssnnnggacg

SEQ ID NO: 553 - DNA, 15 bp, Artificial
  Oligonucleotide
tggttssnnnggacg

SEQ ID NO: 554 - DNA, 15 bp, Artificial
  Oligonucleotide
ttggtssnnnggacg

SEQ ID NO: 555 - DNA, 15 bp, Artificial
  Oligonucleotide
gtacgssnnnggacg

SEQ ID NO: 556 - DNA, 15 bp, Artificial
  Oligonucleotide
gtgtcssnnnggacg

SEQ ID NO: 557 - DNA, 15 bp, Artificial
  Oligonucleotide
cgtacssnnnggacg

SEQ ID NO: 558 - DNA, 15 bp, Artificial
  Oligonucleotide
gtgacssnnnggacg

SEQ ID NO: 559 - DNA, 15 bp, Artificial
  Oligonucleotide
gtcacssnnnggacg

SEQ ID NO: 560 - DNA, 15 bp, Artificial
  Oligonucleotide
gacacssnnnggacg

SEQ ID NO: 561 - DNA, 15 bp, Artificial
  Oligonucleotide
ctgtgssnnnggacg

SEQ ID NO: 562 - DNA, 15 bp, Artificial
  Oligonucleotide
cagtgssnnnggacg

SEQ ID NO: 563 - DNA, 15 bp, Artificial
  Oligonucleotide
cactgssnnnggacg

SEQ ID NO: 564 - DNA, 15 bp, Artificial
  Oligonucleotide
ccatgssnnnggacg

SEQ ID NO: 565 - DNA, 15 bp, Artificial
  Oligonucleotide
catggssnnnggacg

SEQ ID NO: 566 - DNA, 15 bp, Artificial
  Oligonucleotide
cacagssnnnggacg

SEQ ID NO: 567 - DNA, 15 bp, Artificial
  Oligonucleotide
ggttcssnnnggacg

SEQ ID NO: 568 - DNA, 15 bp, Artificial
  Oligonucleotide
gttccssnnnggacg

SEQ ID NO: 569 - DNA, 15 bp, Artificial
  Oligonucleotide
gaaccssnnnggacg

SEQ ID NO: 570 - DNA, 15 bp, Artificial
  Oligonucleotide
ggaacssnnnggacg

SEQ ID NO: 571 - DNA, 15 bp, Artificial
  Oligonucleotide
ccttgssnnnggacg

SEQ ID NO: 572 - DNA, 15 bp, Artificial
  Oligonucleotide
cttggssnnnggacg

SEQ ID NO: 573 - DNA, 15 bp, Artificial
  Oligonucleotide
caaggssnnnggacg

SEQ ID NO: 574 - DNA, 15 bp, Artificial
  Oligonucleotide
ccaagssnnnggacg

SEQ ID NO: 575 - DNA, 15 bp, Artificial
  Oligonucleotide
cgtttssnnnggacg

SEQ ID NO: 576 - DNA, 15 bp, Artificial
  Oligonucleotide
atcgassnnnggacg

SEQ ID NO: 577 - DNA, 15 bp, Artificial
  Oligonucleotide
tcgatssnnnggacg

SEQ ID NO: 578 - DNA, 15 bp, Artificial
  Oligonucleotide
gtagcssnnnggacg

SEQ ID NO: 579 - DNA, 15 bp, Artificial
  Oligonucleotide
gctacssnnnggacg

SEQ ID NO: 580 - DNA, 15 bp, Artificial
  Oligonucleotide
tgtgassnnnggacg

SEQ ID NO: 581 - DNA, 15 bp, Artificial
  Oligonucleotide
tgtcassnnnggacg

SEQ ID NO: 582 - DNA, 15 bp, Artificial
  Oligonucleotide
tgacassnnnggacg

SEQ ID NO: 583 - DNA, 15 bp, Artificial
  Oligonucleotide
tcacassnnnggacg

SEQ ID NO: 584 - DNA, 15 bp, Artificial
  Oligonucleotide
tacgtssnnnggacg

SEQ ID NO: 585 - DNA, 15 bp, Artificial
  Oligonucleotide
acgtassnnnggacg

SEQ ID NO: 586 - DNA, 15 bp, Artificial
  Oligonucleotide
attgcssnnnggacg

SEQ ID NO: 587 - DNA, 15 bp, Artificial
  Oligonucleotide
aatgcssnnnggacg

SEQ ID NO: 588 - DNA, 15 bp, Artificial
  Oligonucleotide
gcattssnnnggacg

SEQ ID NO: 589 - DNA, 15 bp, Artificial
  Oligonucleotide
gcaatssnnnggacg

SEQ ID NO: 590 - DNA, 15 bp, Artificial
  Oligonucleotide
actgtssnnnggacg

SEQ ID NO: 591 - DNA, 15 bp, Artificial
  Oligonucleotide
atggtssnnnggacg

SEQ ID NO: 592 - DNA, 15 bp, Artificial
  Oligonucleotide
acagtssnnnggacg

SEQ ID NO: 593 - DNA, 15 bp, Artificial
  Oligonucleotide
acactssnnnggacg

SEQ ID NO: 594 - DNA, 15 bp, Artificial
  Oligonucleotide
accatssnnnggacg

SEQ ID NO: 595 - DNA, 15 bp, Artificial
  Oligonucleotide
agtgtssnnnggacg

SEQ ID NO: 596 - DNA, 15 bp, Artificial
  Oligonucleotide
ttggassnnnggacg

SEQ ID NO: 597 - DNA, 15 bp, Artificial
  Oligonucleotide
ttccassnnnggacg

SEQ ID NO: 598 - DNA, 15 bp, Artificial
  Oligonucleotide
tggaassnnnggacg

SEQ ID NO: 599 - DNA, 15 bp, Artificial
  Oligonucleotide
tccaassnnnggacg

SEQ ID NO: 600 - DNA, 15 bp, Artificial
  Oligonucleotide
gctttssnnnggacg

SEQ ID NO: 601 - DNA, 15 bp, Artificial
  Oligonucleotide
gagtgssnnnggacg

SEQ ID NO: 602 - DNA, 15 bp, Artificial
  Oligonucleotide
gtctgssnnnggacg

SEQ ID NO: 603 - DNA, 15 bp, Artificial
  Oligonucleotide
gactgssnnnggacg

SEQ ID NO: 604 - DNA, 15 bp, Artificial
  Oligonucleotide
ggatgssnnnggacg

SEQ ID NO: 605 - DNA, 15 bp, Artificial
  Oligonucleotide
gatggssnnnggacg

SEQ ID NO: 606 - DNA, 15 bp, Artificial
  Oligonucleotide
ctacgssnnnggacg

SEQ ID NO: 607 - DNA, 15 bp, Artificial
  Oligonucleotide
cgtagssnnnggacg

SEQ ID NO: 608 - DNA, 15 bp, Artificial
  Oligonucleotide
gtgagssnnnggacg

SEQ ID NO: 609 - DNA, 15 bp, Artificial
  Oligonucleotide
gtcagssnnnggacg

SEQ ID NO: 610 - DNA, 15 bp, Artificial
  Oligonucleotide
gacagssnnnggacg

SEQ ID NO: 611 - DNA, 15 bp, Artificial
  Oligonucleotide
ctgtcssnnnggacg

SEQ ID NO: 612 - DNA, 15 bp, Artificial
  Oligonucleotide
cagtcssnnnggacg

SEQ ID NO: 613 - DNA, 15 bp, Artificial
  Oligonucleotide
cactcssnnnggacg

SEQ ID NO: 614 - DNA, 15 bp, Artificial
  Oligonucleotide
ccatcssnnnggacg

SEQ ID NO: 615 - DNA, 15 bp, Artificial
  Oligonucleotide
catccssnnnggacg

SEQ ID NO: 616 - DNA, 15 bp, Artificial
  Oligonucleotide
ctgacssnnnggacg

SEQ ID NO: 617 - DNA, 15 bp, Artificial
  Oligonucleotide
cagacssnnnggacg

SEQ ID NO: 618 - DNA, 15 bp, Artificial
  Oligonucleotide
ctcacssnnnggacg

SEQ ID NO: 619 - DNA, 15 bp, Artificial
  Oligonucleotide
aggttssnnnggacg

SEQ ID NO: 620 - DNA, 15 bp, Artificial
  Oligonucleotide
accttssnnnggacg

SEQ ID NO: 621 - DNA, 15 bp, Artificial
  Oligonucleotide
aaggtssnnnggacg

SEQ ID NO: 622 - DNA, 15 bp, Artificial
  Oligonucleotide
aacctssnnnggacg

SEQ ID NO: 623 - DNA, 15 bp, Artificial
  Oligonucleotide
tgctassnnnggacg

SEQ ID NO: 624 - DNA, 15 bp, Artificial
  Oligonucleotide
tagcassnnnggacg

SEQ ID NO: 625 - DNA, 15 bp, Artificial
  Oligonucleotide
gaaggssnnnggacg

SEQ ID NO: 626 - DNA, 15 bp, Artificial
  Oligonucleotide
tttcgssnnnggacg

SEQ ID NO: 627 - DNA, 15 bp, Artificial
  Oligonucleotide
ggaagssnnnggacg

SEQ ID NO: 628 - DNA, 15 bp, Artificial
  Oligonucleotide
ccttcssnnnggacg

SEQ ID NO: 629 - DNA, 15 bp, Artificial
  Oligonucleotide
cttccssnnnggacg

SEQ ID NO: 630 - DNA, 15 bp, Artificial
  Oligonucleotide
tcgtassnnnggacg

SEQ ID NO: 631 - DNA, 15 bp, Artificial
  Oligonucleotide
tacgassnnnggacg

SEQ ID NO: 632 - DNA, 15 bp, Artificial
  Oligonucleotide
gctagssnnnggacg

SEQ ID NO: 633 - DNA, 15 bp, Artificial
  Oligonucleotide
ctagcssnnnggacg

SEQ ID NO: 634 - DNA, 15 bp, Artificial
  Oligonucleotide
tctgtssnnnggacg

SEQ ID NO: 635 - DNA, 15 bp, Artificial
  Oligonucleotide
tgagtssnnnggacg

SEQ ID NO: 636 - DNA, 15 bp, Artificial
  Oligonucleotide
tcagtssnnnggacg

SEQ ID NO: 637 - DNA, 15 bp, Artificial
  Oligonucleotide
tgtctssnnnggacg

SEQ ID NO: 638 - DNA, 15 bp, Artificial
  Oligonucleotide
tgactssnnnggacg

SEQ ID NO: 639 - DNA, 15 bp, Artificial
  Oligonucleotide
tcactssnnnggacg

SEQ ID NO: 640 - DNA, 15 bp, Artificial
  Oligonucleotide
tggatssnnnggacg

SEQ ID NO: 641 - DNA, 15 bp, Artificial
  Oligonucleotide
tccatssnnnggacg

SEQ ID NO: 642 - DNA, 15 bp, Artificial
  Oligonucleotide
atggassnnnggacg

SEQ ID NO: 643 - DNA, 15 bp, Artificial
  Oligonucleotide
agtcassnnnggacg

SEQ ID NO: 644 - DNA, 15 bp, Artificial
  Oligonucleotide
agacassnnnggacg

SEQ ID NO: 645 - DNA, 15 bp, Artificial
  Oligonucleotide
gagtcssnnnggacg

SEQ ID NO: 646 - DNA, 15 bp, Artificial
  Oligonucleotide
gactcssnnnggacg

SEQ ID NO: 647 - DNA, 15 bp, Artificial
  Oligonucleotide
ggatcssnnnggacg

SEQ ID NO: 648 - DNA, 15 bp, Artificial
  Oligonucleotide
gatccssnnnggacg

SEQ ID NO: 649 - DNA, 15 bp, Artificial
  Oligonucleotide
gagacssnnnggacg

SEQ ID NO: 650 - DNA, 15 bp, Artificial
  Oligonucleotide
cagagssnnnggacg

SEQ ID NO: 651 - DNA, 15 bp, Artificial
  Oligonucleotide
ctctgssnnnggacg

SEQ ID NO: 652 - DNA, 15 bp, Artificial
  Oligonucleotide
ctgagssnnnggacg

SEQ ID NO: 653 - DNA, 15 bp, Artificial
  Oligonucleotide
ctcagssnnnggacg

SEQ ID NO: 654 - DNA, 15 bp, Artificial
  Oligonucleotide
cgattssnnnggacg

SEQ ID NO: 655 - DNA, 15 bp, Artificial
  Oligonucleotide
cgaatssnnnggacg

SEQ ID NO: 656 - DNA, 15 bp, Artificial
  Oligonucleotide
attcgssnnnggacg

SEQ ID NO: 657 - DNA, 15 bp, Artificial
  Oligonucleotide
aatcgssnnnggacg

SEQ ID NO: 658 - DNA, 15 bp, Artificial
  Oligonucleotide
gtaccssnnnggacg

SEQ ID NO: 659 - DNA, 15 bp, Artificial
  Oligonucleotide
ggtacssnnnggacg

SEQ ID NO: 660 - DNA, 15 bp, Artificial
  Oligonucleotide
tgttgssnnnggacg

SEQ ID NO: 661 - DNA, 15 bp, Artificial
  Oligonucleotide
ttgtgssnnnggacg

SEQ ID NO: 662 - DNA, 15 bp, Artificial
  Oligonucleotide
caacassnnnggacg

SEQ ID NO: 663 - DNA, 15 bp, Artificial
  Oligonucleotide
cacaassnnnggacg

SEQ ID NO: 664 - DNA, 15 bp, Artificial
  Oligonucleotide
gtgttssnnnggacg

SEQ ID NO: 665 - DNA, 15 bp, Artificial
  Oligonucleotide
gttgtssnnnggacg

SEQ ID NO: 666 - DNA, 15 bp, Artificial
  Oligonucleotide
tagctssnnnggacg

SEQ ID NO: 667 - DNA, 15 bp, Artificial
  Oligonucleotide
aacacssnnnggacg

SEQ ID NO: 668 - DNA, 15 bp, Artificial
  Oligonucleotide
acaacssnnnggacg

SEQ ID NO: 669 - DNA, 15 bp, Artificial
  Oligonucleotide
agctassnnnggacg

SEQ ID NO: 670 - DNA, 15 bp, Artificial
  Oligonucleotide
tttggssnnnggacg

SEQ ID NO: 671 - DNA, 15 bp, Artificial
  Oligonucleotide
tctgassnnnggacg

SEQ ID NO: 672 - DNA, 15 bp, Artificial
  Oligonucleotide
tgagassnnnggacg

SEQ ID NO: 673 - DNA, 15 bp, Artificial
  Oligonucleotide
tcagassnnnggacg

SEQ ID NO: 674 - DNA, 15 bp, Artificial
  Oligonucleotide
tctcassnnnggacg

SEQ ID NO: 675 - DNA, 15 bp, Artificial
  Oligonucleotide
tggtassnnnggacg

SEQ ID NO: 676 - DNA, 15 bp, Artificial
  Oligonucleotide
taccassnnnggacg

SEQ ID NO: 677 - DNA, 15 bp, Artificial
  Oligonucleotide
agagtssnnnggacg

SEQ ID NO: 678 - DNA, 15 bp, Artificial
  Oligonucleotide
agtctssnnnggacg

SEQ ID NO: 679 - DNA, 15 bp, Artificial
  Oligonucleotide
actctssnnnggacg

SEQ ID NO: 680 - DNA, 15 bp, Artificial
  Oligonucleotide
atcctssnnnggacg

SEQ ID NO: 681 - DNA, 15 bp, Artificial
  Oligonucleotide
agactssnnnggacg

SEQ ID NO: 682 - DNA, 15 bp, Artificial
  Oligonucleotide
aggatssnnnggacg

SEQ ID NO: 683 - DNA, 15 bp, Artificial
  Oligonucleotide
catgtssnnnggacg

SEQ ID NO: 684 - DNA, 15 bp, Artificial
  Oligonucleotide
cacatssnnnggacg

SEQ ID NO: 685 - DNA, 15 bp, Artificial
  Oligonucleotide
atgtgssnnnggacg

SEQ ID NO: 686 - DNA, 15 bp, Artificial
  Oligonucleotide
acatgssnnnggacg

SEQ ID NO: 687 - DNA, 15 bp, Artificial
  Oligonucleotide
gagagssnnnggacg

SEQ ID NO: 688 - DNA, 15 bp, Artificial
  Oligonucleotide
ctctcssnnnggacg

SEQ ID NO: 689 - DNA, 15 bp, Artificial
  Oligonucleotide
gtaggssnnnggacg

SEQ ID NO: 690 - DNA, 15 bp, Artificial
  Oligonucleotide
ttacgssnnnggacg

SEQ ID NO: 691 - DNA, 15 bp, Artificial
  Oligonucleotide
taacgssnnnggacg

SEQ ID NO: 692 - DNA, 15 bp, Artificial
  Oligonucleotide
ggtagssnnnggacg

SEQ ID NO: 693 - DNA, 15 bp, Artificial
  Oligonucleotide
tgttcssnnnggacg

SEQ ID NO: 694 - DNA, 15 bp, Artificial
  Oligonucleotide
ttgtcssnnnggacg

SEQ ID NO: 695 - DNA, 15 bp, Artificial
  Oligonucleotide
ctaccssnnnggacg

SEQ ID NO: 696 - DNA, 15 bp, Artificial
  Oligonucleotide
cctacssnnnggacg

SEQ ID NO: 697 - DNA, 15 bp, Artificial
  Oligonucleotide
ttgacssnnnggacg

SEQ ID NO: 698 - DNA, 15 bp, Artificial
  Oligonucleotide
ttcacssnnnggacg

SEQ ID NO: 699 - DNA, 15 bp, Artificial
  Oligonucleotide
tgaacssnnnggacg

SEQ ID NO: 700 - DNA, 15 bp, Artificial
  Oligonucleotide
tcaacssnnnggacg

SEQ ID NO: 701 - DNA, 15 bp, Artificial
  Oligonucleotide
cgttassnnnggacg

SEQ ID NO: 702 - DNA, 15 bp, Artificial
  Oligonucleotide
gttgassnnnggacg

SEQ ID NO: 703 - DNA, 15 bp, Artificial
  Oligonucleotide
gttcassnnnggacg

SEQ ID NO: 704 - DNA, 15 bp, Artificial
  Oligonucleotide
gaacassnnnggacg

SEQ ID NO: 705 - DNA, 15 bp, Artificial
  Oligonucleotide
cgtaassnnnggacg

SEQ ID NO: 706 - DNA, 15 bp, Artificial
  Oligonucleotide
gtgaassnnnggacg

SEQ ID NO: 707 - DNA, 15 bp, Artificial
  Oligonucleotide
gtcaassnnnggacg

SEQ ID NO: 708 - DNA, 15 bp, Artificial
  Oligonucleotide
gacaassnnnggacg

SEQ ID NO: 709 - DNA, 15 bp, Artificial
  Oligonucleotide
ctgttssnnnggacg

SEQ ID NO: 710 - DNA, 15 bp, Artificial
  Oligonucleotide
cagttssnnnggacg

SEQ ID NO: 711 - DNA, 15 bp, Artificial
  Oligonucleotide
cacttssnnnggacg

SEQ ID NO: 712 - DNA, 15 bp, Artificial
  Oligonucleotide
ccattssnnnggacg

SEQ ID NO: 713 - DNA, 15 bp, Artificial
  Oligonucleotide
cttgtssnnnggacg

SEQ ID NO: 714 - DNA, 15 bp, Artificial
  Oligonucleotide
caagtssnnnggacg

SEQ ID NO: 715 - DNA, 15 bp, Artificial
  Oligonucleotide
caactssnnnggacg

SEQ ID NO: 716 - DNA, 15 bp, Artificial
  Oligonucleotide
ccaatssnnnggacg

SEQ ID NO: 717 - DNA, 15 bp, Artificial
  Oligonucleotide
agttgssnnnggacg

SEQ ID NO: 718 - DNA, 15 bp, Artificial
  Oligonucleotide
acttgssnnnggacg

SEQ ID NO: 719 - DNA, 15 bp, Artificial
  Oligonucleotide
attggssnnnggacg

SEQ ID NO: 720 - DNA, 15 bp, Artificial
  Oligonucleotide
aatggssnnnggacg

SEQ ID NO: 721 - DNA, 15 bp, Artificial
  Oligonucleotide
aacagssnnnggacg

SEQ ID NO: 722 - DNA, 15 bp, Artificial
  Oligonucleotide
tatgcssnnnggacg

SEQ ID NO: 723 - DNA, 15 bp, Artificial
  Oligonucleotide
gcatassnnnggacg

SEQ ID NO: 724 - DNA, 15 bp, Artificial
  Oligonucleotide
tttccssnnnggacg

SEQ ID NO: 725 - DNA, 15 bp, Artificial
  Oligonucleotide
tctctssnnnggacg

SEQ ID NO: 726 - DNA, 15 bp, Artificial
  Oligonucleotide
agagassnnnggacg

SEQ ID NO: 727 - DNA, 15 bp, Artificial
  Oligonucleotide
ttagcssnnnggacg

SEQ ID NO: 728 - DNA, 15 bp, Artificial
  Oligonucleotide
taagcssnnnggacg

SEQ ID NO: 729 - DNA, 15 bp, Artificial
  Oligonucleotide
gcttassnnnggacg

SEQ ID NO: 730 - DNA, 15 bp, Artificial
  Oligonucleotide
gctaassnnnggacg

SEQ ID NO: 731 - DNA, 15 bp, Artificial
  Oligonucleotide
taggtssnnnggacg

SEQ ID NO: 732 - DNA, 15 bp, Artificial
  Oligonucleotide
tacctssnnnggacg

SEQ ID NO: 733 - DNA, 15 bp, Artificial
  Oligonucleotide
aggtassnnnggacg

SEQ ID NO: 734 - DNA, 15 bp, Artificial
  Oligonucleotide
acctassnnnggacg

SEQ ID NO: 735 - DNA, 15 bp, Artificial
  Oligonucleotide
tgatgssnnnggacg

SEQ ID NO: 736 - DNA, 15 bp, Artificial
  Oligonucleotide
tcatgssnnnggacg

SEQ ID NO: 737 - DNA, 15 bp, Artificial
  Oligonucleotide
catcassnnnggacg

SEQ ID NO: 738 - DNA, 15 bp, Artificial
  Oligonucleotide
cgtatssnnnggacg

SEQ ID NO: 739 - DNA, 15 bp, Artificial
  Oligonucleotide
gatgtssnnnggacg

SEQ ID NO: 740 - DNA, 15 bp, Artificial
  Oligonucleotide
gtgatssnnnggacg

SEQ ID NO: 741 - DNA, 15 bp, Artificial
  Oligonucleotide
gtcatssnnnggacg

SEQ ID NO: 742 - DNA, 15 bp, Artificial
  Oligonucleotide
gacatssnnnggacg

SEQ ID NO: 743 - DNA, 15 bp, Artificial
  Oligonucleotide
tcttgssnnnggacg

SEQ ID NO: 744 - DNA, 15 bp, Artificial
  Oligonucleotide
ttctgssnnnggacg

SEQ ID NO: 745 - DNA, 15 bp, Artificial
  Oligonucleotide
ctaggssnnnggacg

SEQ ID NO: 746 - DNA, 15 bp, Artificial
  Oligonucleotide
cctagssnnnggacg

SEQ ID NO: 747 - DNA, 15 bp, Artificial
  Oligonucleotide
ttgagssnnnggacg

SEQ ID NO: 748 - DNA, 15 bp, Artificial
  Oligonucleotide
ttcagssnnnggacg

SEQ ID NO: 749 - DNA, 15 bp, Artificial
  Oligonucleotide
tgaagssnnnggacg

SEQ ID NO: 750 - DNA, 15 bp, Artificial
  Oligonucleotide
tcaagssnnnggacg

SEQ ID NO: 751 - DNA, 15 bp, Artificial
  Oligonucleotide
cttgassnnnggacg

SEQ ID NO: 752 - DNA, 15 bp, Artificial
  Oligonucleotide
caagassnnnggacg

SEQ ID NO: 753 - DNA, 15 bp, Artificial
  Oligonucleotide
cttcassnnnggacg

SEQ ID NO: 754 - DNA, 15 bp, Artificial
  Oligonucleotide
ctgaassnnnggacg

SEQ ID NO: 755 - DNA, 15 bp, Artificial
  Oligonucleotide
cagaassnnnggacg

SEQ ID NO: 756 - DNA, 15 bp, Artificial
  Oligonucleotide
ctcaassnnnggacg

SEQ ID NO: 757 - DNA, 15 bp, Artificial
  Oligonucleotide
gagttssnnnggacg

SEQ ID NO: 758 - DNA, 15 bp, Artificial
  Oligonucleotide
gtcttssnnnggacg

SEQ ID NO: 759 - DNA, 15 bp, Artificial
  Oligonucleotide
gacttssnnnggacg

SEQ ID NO: 760 - DNA, 15 bp, Artificial
  Oligonucleotide
gaagtssnnnggacg

SEQ ID NO: 761 - DNA, 15 bp, Artificial
  Oligonucleotide
gttctssnnnggacg

SEQ ID NO: 762 - DNA, 15 bp, Artificial
  Oligonucleotide
gaactssnnnggacg

SEQ ID NO: 763 - DNA, 15 bp, Artificial
  Oligonucleotide
ggaatssnnnggacg

SEQ ID NO: 764 - DNA, 15 bp, Artificial
  Oligonucleotide
gctatssnnnggacg

SEQ ID NO: 765 - DNA, 15 bp, Artificial
  Oligonucleotide
tcctassnnnggacg

SEQ ID NO: 766 - DNA, 15 bp, Artificial
  Oligonucleotide
taggassnnnggacg

SEQ ID NO: 767 - DNA, 15 bp, Artificial
  Oligonucleotide
tatcgssnnnggacg

SEQ ID NO: 768 - DNA, 15 bp, Artificial
  Oligonucleotide
tgatcssnnnggacg

SEQ ID NO: 769 - DNA, 15 bp, Artificial
  Oligonucleotide
tcatcssnnnggacg

SEQ ID NO: 770 - DNA, 15 bp, Artificial
  Oligonucleotide
catctssnnnggacg

SEQ ID NO: 771 - DNA, 15 bp, Artificial
  Oligonucleotide
ctgatssnnnggacg

SEQ ID NO: 772 - DNA, 15 bp, Artificial
  Oligonucleotide
cagatssnnnggacg

SEQ ID NO: 773 - DNA, 15 bp, Artificial
  Oligonucleotide
ctcatssnnnggacg

SEQ ID NO: 774 - DNA, 15 bp, Artificial
  Oligonucleotide
tgtacssnnnggacg

SEQ ID NO: 775 - DNA, 15 bp, Artificial
  Oligonucleotide
tacacssnnnggacg

SEQ ID NO: 776 - DNA, 1645 bp, Homo sapiens
tcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccgggtgagcagtaggggggctttctcctgctgcttatttgacctccctataaccccatgagatgtgcaaagtaaatgggtttaactattgcacagttgaaaaaactgaagcttacgaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgccgctgacccctgggcccacctcttaccgatttcttccatactactacccatccacctctcatcacatttccggcgggaatctccttactgctcccactcagtttccttttctctggctttgggacctcttaacctgtggcttctcctcccacctcctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcactt

SEQ ID NO: 777 - DNA, 20 bp, Artificial
  Oligonucleotide
cagagttgaaagtcaggatc

SEQ ID NO: 778 - DNA, 20 bp, Artificial
  Oligonucleotide
gggtgcagttatgcctcttc

SEQ ID NO: 779 - DNA, 1292 bp, Escherichia coli
atgtttgaaccaatggaacttaccaatgacgcggtgattaaagtcatcggcgtcggcggcggcggcggtaatgctgttgaacacatggtgcgcgagcgcattgaaggtgttgaattcttcgcggtaaataccgatgcacaagcgctgcgtaaaacagcggttggacagacgattcaaatcggtagcggtatcaccaaaggactgggcgctggcgctaatccagaagttggccgcaatgcggctgatgaggatcgcgatgcattgcgtgcggcgctggaaggtgcagacatggtctttattgctgcgggtatgggtggtggtaccggtacaggtgcggcaccagtcgtcgctgaagtggcaaaagatttgggtatcctgaccgttgctgtcgtcactaagcctttcaactttgaaggcaagaagcgtatggcattcgcggagcaggggatcactgaactgtccaagcatgtgaactctctgatcactatcccgaacgacaaactgctgaaagttctgggccgcggtatctccctgctggatgcgtttggcgcagcgaacgatgtactgaaaggcgctgtgcaaggtatcgctgaactgattactcgtccgggtttgatgaacgtggactttgcagacgtacgcaccgtaatgtctgagatgggccacgcaatgatgggttctggcgtggcgagcggtgaagaccgtgcggaagaagctgctgaaatggctatctcttctccgctgctggaagatatcgacctgtctggcgcgcgcggcgtgctggttaacatcacggcgggcttcgacctgcgtctggatgagttcgaaacggtaggtaacaccatccgtgcatttgcttccgacaacgcgactgtggttatcggtacttctcttgacccggatatgaatgacgagctgcgcgtaaccgttgttgcgacaggtatcggcatggacaaacgtcctgaaatcactctggtgaccaataagcaggttcagcagccagtgatggatcgctaccagcagcatgggatggctccgctgacccaagagcagaagccggttgctaaagtcgtgaatgacaatgcgccgcaaactgcgaaagagccggattatctggatatcccagcattcctgcgtaagcaagctgattaaggatccgcaagggcgaattccagcacactggcggccgttactagtggatccgagctcggtaccaagcttgatgcatagcttgagtattctatagtgtcacctaaatagcttggcgtaatcatggtcatagctgtttcctg

SEQ ID NO: 780 - DNA, 15 bp, Artificial
  Oligonucleotide
actgggttgctgtcg

SEQ ID NO: 781 - DNA, 15 bp, Artificial
  Oligonucleotide
cgacassnnnccagt

SEQ ID NO: 782 - DNA, 15 bp, Artificial
  Oligonucleotide
ggacgataccgatgc

SEQ ID NO: 783 - DNA, 15 bp, Artificial
  Oligonucleotide
gcatcssnnncgtcc

SEQ ID NO: 784 - DNA, 592 bp, Artificial
  DNA Sequencing Reaction PCR Product
aaggggggnnnngctntcgttgttgnangctgaagcgtatggcattcgcggagcaggggatcactgaactgtccaagcatgtggactctctgatcactatcccgaacgacaaactgctgaaagttctgggccgcggtatctccctgctggatgcgtttggcgcagcgaacgatgtactgaaaggcgctgtgcaaggtatcgctgaactgattactcgtccgggtttgatgaacgtggactttgcagacgtacgcaccgtaatgtctgagatgggctacgcaatgatgggttctggcgtggcgagcggtgaagaccgtgcggaagaagctgctgaaatggctatctcttctccgctgctggaagatatcgacctgtctggcgcgcgcggcgtgctggttaacatcacggcgggcttcgacctgcgtctggatgagttcgaaacggtaggtaacaccatccgtgcatttgcttccgacaacgcgactgtggttatcggtacttctcttgacccggatatgaatgacgagctgcgcgtaaccgttgttgcgacaggtatcggcatggacaaacgtnctgaaatcactctggtgac

SEQ ID NO: 785 - DNA, 613 bp, Artificial
  DNA Sequencing Reaction PCR Product
gnnnnnnnnnangagggnnngananggngatnnttggccctttggacagacgatncanatcgntagcggtatcaccnaaggactgggncgctggcgctnntccagaaagttggccgcaatgcggctgatgaggatcgatgcattgcgtgcggcgctggaaggtgcagacatggtctttattgctgcgggtatgggtggtggtaccggtacaggtgcagcaccagtcgtcgctgaagtggcaaaagatttgggtatcgaccgttgctgtcgtcactaagcctttcaactttgaaggcaagaagcgtatggcattcgcggagcaggggatcactgagctgtccaagcatgtggactctctgatcactatcccgaacgaaactgctgaaagttctgggccgcggtatctccctgctggatgcgtttggcgcagcgaacgatgtactgaaaggcgctgtgcaaggtatcgctgaactgattactcgtccgggtttgatacgtggactttgcagacgtacgcaccgtaatgtctgagatgggctacgcaatgatgggttctggcgtggcgagcggtgaagaccgtgcggaagaagctgctgaaatggctatctcttct

SEQ ID NO: 786 - DNA, 1302 bp, Escherichia coli
ccgcatgggtgcgaacttcctgaaagttgttaaccagatcaaaacccgtctgggcgcgaacccggttccgctgcagctggcgattggtgctgaagaacatttcaccggtgttgttgacctggtgaaaatgaaagctatcaactggaacgacgctgaccagggcgtaaccttcgaatacgaagatatcccggcagacatggttgaactggctaacgaatggcaccagaacctgatcgaatccgcagctgaagcttctgaagagctgatggaaaaatacctgggtggtgaagaactgactgaagcagaaatcaaaggtgctctgcgtcagcgcgttctgaacaacgaaatcatcctggtaacctgtggttctgcgttcaagaacaaaggtgttcaggcgatgctggatgcggtaattgattacctgccatccccggttgacgtacctgcgatcaacggtatcctggacgacggtaaagacactccggctgaacgtcacgcaagtgatgacgagccgttctctgcactggcgttcaaaatcgctaccgacccgtttgttggtaacctgaccttcttccgtgtttactccggtgtggttaactctggtgataccgtactgaactccgtgaaagctgcacgtgagcgtttcggtcgtatcgttcagatgcacgctaacaaacgtgaagagatcaaagaagttcgcgcgggcgacatcgctgctgctatcggtctgaaagacgtaaccactggtgacaccctgtgtgacccggatgcgccgatcattctggaacgtatggaattccctgagccggtaatctccatcgcagttgaaccgaaaaccaaagctgaccaggaaaaaatgggtctggctctgggccgtctggctaaagaagacccgtctttccgtgtatggactgacgaagaatctaaccagaccatcatcgcgggtatgggcgaactgcacctcgacatcatcgttgaccgtatgaagcgtgaattcaacgttgaagcgaacgtaggtaaaccgcaggttgcttaccgtgaaactatccgccagaaagttaccgatgttgaaggtaaacacgcgaaacagtctggtggtcgtggtcagtatggtcatgttgttatcgacatgtacccgctggagccgggttcaaacccgaaaggctacgagttcatcaacgacattaaaggtggtgtaatccctggcgaatacatcccggccgttgataaaggtatccaggaacagctgaaagcaggtccgctggcaggctacccggtagtagacatgggtattcgtctgca

SEQ ID NO: 787 - DNA, 15 bp, Artificial
  Oligonucleotide
ggacgattggtgctg

SEQ ID NO: 788 - DNA, 15 bp, Artificial
  Oligonucleotide
cagcassnnncgtcc

SEQ ID NO: 789 - DNA, 609 bp, Artificial
  DNA Sequencing Reaction PCR Product
agagncgaantngntgtgctttncgttgnagtaaagctatcaacttgaacgacgctgaccagggcgtaaccttcgaatacgaagatatcccggcagacatggttgaactggctaacgaatggcaccagaacctgatcgaatccgcagctgaagcttctgaagagctgatggaaaaatacctgggtggtgaagaactgactgaagcagaaatcaaggtgctctgcgtcagcgcgttctgaacaacgaaatcctggtaacctgtggttctgcgttcaagaacaaaggtgttcaggcgatgctggatgcggtaattgattacctgccatccccggttgacgtacctgcgatcaacggtatcctggacgacggtagacactccggctgaacgtcacgcaagtgatgacgagccgttctctgcactggcgttcaaaatcgctccgacccgtttgttggtaacctgaccttcttcgtgtttactccggtgtggttatctggtgataccgactgaactccgtgaaagctgcacgtgagcgtttcggtcgatcgttcaaatgcacnttacaaacgtgaagagatcaaagaagtcgcgcggcgacatcgctgctgcta

SEQ ID NO: 790 - DNA, 3173 bp, Escherichia coli
ggtaccagttgaagtccgtccggttcgtcgtaatgctctggcaatgcgttggatcgttgaagctgctcgtaaacgcggtgataaatccatggctctgcgcctggcgaacgaactttctgatgctgcagaaaacaaaggtactgcagttaagaaacgtgaagacgttcaccgtatggccgaagccaacaaggcgttcgcacactaccgttggttatcccttcggagttttagtcaccaggcgggcgcttccagtaagcagcccgctttgggctacttaaattgaacgcctaaaagataaacgaggaaacaaatggctcgtacaacacccatcgcacgctaccgtaacatcggtatcagtgcgcacatcgacgccggtaaaaccactactaccgaacgtattctgttctacaccggtgtaaaccataaaatcggtgaagttcatgacggcgctgcaaccatggactggatggagcaggagcaggaacgtggtattaccatcacttccgctgcgactactgcattctggtctggtatggctaagcagtatgagccgcatcgcatcaacatcatcgacaccccggggcacgttgacttcacaatcgaagtagaacgttccatgcgtgttctcgatggtgcggtaatggtttactgcgcagttggtggtgttcagccgcagtctgaaaccgtatggcgtcaggcaaacaaatataaagttccgcgcattgcgttcgttaacaaaatggaccgcatgggtgcgaacttcctgaaagttgttaaccagatcaaaacccgtctgggcgcgaacccggttccgctgcagctggcgattggtgctgaagaacatttcaccggtgttgttgacctggtgaaaatgaaagctatcaactggaacgacgctgaccagggcgtaaccttcgaatacgaagatatcccggcagacatggttgaactggctaacgaatggcaccagaacctgatcgaatccgcagctgaagcttctgaagagctgatggaaaaatacctgggtggtgaagaactgactgaagcagaaatcaaaggtgctctgcgtcagcgcgttctgaacaacgaaatcatcctggtaacctgtggttctgcgttcaagaacaaaggtgttcaggcgatgctggatgcggtaattgattacctgccatccccggttgacgtacctgcgatcaacggtatcctggacgacggtaaagacactccggctgaacgtcacgcaagtgatgacgagccgttctctgcactggcgttcaaaatcgctaccgacccgtttgttggtaacctgaccttcttccgtgtttactccggtgtggttaactctggtgataccgtactgaactccgtgaaagctgcacgtgagcgtttcggtcgtatcgttcagatgcacgctaacaaacgtgaagagatcaaagaagttcgcgcgggcgacatcgctgctgctatcggtctgaaagacgtaaccactggtgacaccctgtgtgacccggatgcgccgatcattctggaacgtatggaattccctgagccggtaatctccatcgcagttgaaccgaaaaccaaagctgaccaggaaaaaatgggtctggctctgggccgtctggctaaagaagacccgtctttccgtgtatggactgacgaagaatctaaccagaccatcatcgcgggtatgggcgaactgcacctcgacatcatcgttgaccgtatgaagcgtgaattcaacgttgaagcgaacgtaggtaaaccgcaggttgcttaccgtgaaactatccgccagaaagttaccgatgttgaaggtaaacacgcgaaacagtctggtggtcgtggtcagtatggtcatgttgttatcgacatgtacccgctggagccgggttcaaacccgaaaggctacgagttcatcaacgacattaaaggtggtgtaatccctggcgaatacatcccggccgttgataaaggtatccaggaacagctgaaagcaggtccgctggcaggctacccggtagtagacatgggtattcgtctgcacttcggttcttaccatgacgttgactcctctgaactggcgtttaaactggctgcttctatcgcctttaaagaaggctttaagaaagcgaaaccagttctgcttgagccgatcatgaaggttgaagtagaaactccggaagagaacaccggtgacgttatcggtgacttgagccgtcgtcgtggtatgctcaaaggtcaggaatctgaagttactggcgttaagatccacgctgaagtaccgctgtctgaaatgttcggatacgcaactcagctgcgttctctgaccaaaggtcgtgcatcatacactatggaattcctgaagtatgatgaagcgccgagtaacgttgctcaggccgtaattgaagcccgtggtaaataagcctaagggttaataccaaagtcccgtgctctctcctgaaggggagagcactatagtaaggaatatagccgtgtctaaagaaaaatttgaacgtacaaaaccgcacgttaacgttggtactatcggccacgttgaccacggtaaaactactctgaccgctgcaatcaccaccgtactggctaaaacctacggcggtgctgctcgtgcattcgaccagatcgataacgcgccggaagaaaaagctcgtggtatcaccatcaacacttctcacgttgaatacgacaccccgacccgtcactacgcacacgtagactgcccggggcacgccgactatgttaaaaacatgatcaccggtgctgctcagatggacggcgcgatcctggtagttgctgcgactgacggcccgatgccgcagactcgtgagcacatcctgctgggtcgtcaggtaggcgttccgtacatcatcgtgttcctgaacaaatgcgacatggttgatgacgaagagctgctggaactggttgaaatggaagttcgtgaacttctgtctcagtacgacttcccgggcgacgacactccgatcgttcgtggttctgctctgaaagcgctggaaggcgacgcagagtgggaagcgaaaatcctggaactggctggcttcctggattcttatattccggaaccagagcgtgcgattgacaagccgttcctgctgccgatcgaagacgtattctccatctccggtcgtggtacc

SEQ ID NO: 791 - DNA, 15 bp, Artificial
  Oligonucleotide
ggacggattggtgct

SEQ ID NO: 792 - DNA, 15 bp, Artificial
  Oligonucleotide
agcacnnnnncgtcc

SEQ ID NO: 793 - DNA, 15 bp, Artificial
  Oligonucleotide
ggacgattggtgctg

SEQ ID NO: 794 - DNA, 15 bp, Artificial
  Oligonucleotide
cagcannnnncgtcc

SEQ ID NO: 795 - DNA, 15 bp, Artificial
  Oligonucleotide
ggacggtatggcgtc

SEQ ID NO: 796 - DNA, 15 bp, Artificial
  Oligonucleotide
gacgcnnnnncgtcc

SEQ ID NO: 797 - DNA, 15 bp, Artificial
  Oligonucleotide
ggacgtcgaatacga

SEQ ID NO: 798 - DNA, 15 bp, Artificial
  Oligonucleotide
tcgtannnnncgtcc

SEQ ID NO: 799 - DNA, 15 bp, Artificial
  Oligonucleotide
ggacgattggtgctg

SEQ ID NO: 800 - DNA, 15 bp, Artificial
  Oligonucleotide
cagcasnnnncgtcc

SEQ ID NO: 801 - DNA, 15 bp, Artificial
  Oligonucleotide
cagcannnnncgtcc

SEQ ID NO: 802 - DNA, 224 bp, Escherichia coli
gcgttgattggtgctgaggagcatatcgcatctcatcataatgtcgtatctcctggggtgttatacaagatatcgttgttggtgacctgggagaggaattgagttctattaaaccgtcaactatgccggatacatactggattacactgcaggcacgccttatgagagaacgtgccgcagtgacgggttaattatctgaaagaatttgtgaggctgtatcggtt

SEQ ID NO: 803 - DNA, 15 bp, Artificial
  Oligonucleotide
ggacgacagcctcac

SEQ ID NO: 804 - DNA, 15 bp, Artificial
  Oligonucleotide
gtgagssnnncgtcc

SEQ ID NO: 805 - DNA, 2686 bp, Artificial
  pUC19 Plasmid
cttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaag

SEQ ID NO: 806 - DNA, 602 bp, Artificial
  DNA Sequencing Reaction PCR Product
gagnnnngcggantgntagngntntcttngcaatngttcttctggcgaaaactctcaaggatcttaccgctgtcgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgctgtaagcggatgccgggagcagacaagcccgtcagggcgcgtcagcgggtgttggcgggtgtcggggctggcttaactatgcgggatcagacagattgtctgagagtgcacca

SEQ ID NO: 807 - DNA, 618 bp, Artificial
  DNA Sequencing Reaction PCR Product
ggnncgggagtgnatgtngnctnggcagctngagtaagncgngcgacatagcatagcttgnnaagtgctcatcattggaaaacgtncttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttccttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagccggtgttggcgggttgtcggggctggcttaactt

(19) United States
(12) Patent Application Publication
(10) Pub. No.: US 2006/0099584 A1
(43) Pub. Date: May 11, 2006

(54) A METHOD FOR INCREASING THE AFFINITY OF AN OLIGONUCLEOTIDE FOR A TARGET NUCLEIC ACID

(76) Inventors: Daniel Tillett, New South Wales (AU); Torsten Thomas, New South Wales (AU)

Correspondence Address: FISH & NEAVE IP GROUP;ROPES & GRAY LLP, ONE INTERNATIONAL PLACE, BOSTON, MA, 02110-2624, US

(21) Appl. No.: 10/513,076

(22) PCT Filed: Dec. 24, 2002
(86) PCT No.: PCT/AU02/01763

§ 371 Date: Oct. 29, 2004

(30) Foreign Application Priority Data
May 1, 2002 (AU) PS2045

(51) Int. Cl. C12Q 1/68; C12P 19/34

(52) U.S. Cl. 435/6; 435/91.2

(57) ABSTRACT

The present invention relates to the optimization of primer libraries. Shorter primers are annealed to template sequences and extended in order to provide primers having improved specificity. The primers of the invention have utility in DNA amplification and sequencing methods.

### TECHNICAL FIELD OF THE INVENTION

The present invention relates to a method of increasing the affinity of an extendable oligonucleotide (EO) for a target nucleic acid comprising the use of a template oligonucleotide (TO), use of the oligonucleotides of the invention for applications requiring linear and exponential amplification of nucleic acids, and related libraries and kits.

### BACKGROUND OF THE INVENTION

Amplification and sequencing of deoxyribonucleic acid (DNA) has become a standard routine in the last few decades in the fields of biotechnological, agricultural, and medical research and related industries. More recently, the advent of large-scale genome sequencing projects, such as the Human Genome Project, has led to a rapid increase in the number of amplification and sequencing reactions performed.

Several techniques exist for the amplification of specific DNA templates from environmental samples, plant or animal tissue, or purified DNA. Today, the most commonly used method for amplifying a target DNA is the Polymerase Chain Reaction (PCR). Four platform patents (U.S. Pat. Nos. 4,800,159, 4,683,202, 4,683,195 and 4,965,188) issued to Cetus Corporation (Emeryville, Calif.) cover this method. Briefly, PCR comprises the following process: two single-stranded oligonucleotide (primers) complementary to the nucleic acid (template) to be amplified and flanking the region of interest are chosen. After a denaturation step, both primers are annealed to the then single-stranded template. Primer extension is accomplished by a DNA polymerase, which is most often thermostable. The resulting double-stranded nucleic acids are again denatured, thereby doubling the number of single-stranded template molecules for the next cycle. The number of product nucleic acid molecules per template molecule theoretically is 2ⁿ, where n is the number of cycles.

A number of other DNA amplification methods, including self-sustaining sequence replication (eg. Guatelli et al., 1990) and the ligase chain reaction (LCR; eg. Wiedmann et al., 1994) are known and complement or provide an alternative to PCR. Recently, substantial new developments in the field of DNA amplification reached the stage of practical application. For example, Rolling Circle Amplification (Lizardi et al., 1998) can be used for sensitive DNA amplification and protein detection. Other amplification techniques include strand displacement amplification which has been shown to be of equivalent sensitivity to LCR (Little et al., 1999).

The most commonly used technique to sequence DNA was developed by Sanger and colleagues (Sanger et al. 1977). It involves the binding of an oligonucleotide (or primer) to a DNA region of interest on the template. A DNA polymerase is then used to extend the oligonucleotide in the presence of normal deoxyribonucleotides and chain-terminating dideoxyribonucleotides (terminators). The latter nucleotides prevent further elongation of the DNA-strand and, as a result, a mixture of DNA molecules is generated. The length of the DNA generated is determined by the position at which the terminator is incorporated. This mixture of DNA molecules is then separated by size on a suitable matrix (gel-slab or capillary column) and the different fragments are detected by functional groups or markers attached to either the primer or terminator (eg. radioactive atoms or fluorescent dye-molecules). The use of thermostable DNA polymerases and thermo-cycling allows a new primer to be annealed to the template DNA and extended, leading to a linear amplification of sequencing signal with cycle number.

The amplification or sequencing of a specific DNA region requires one or more specific oligonucleotide primers. In order to provide specificity, the primer(s) must be of sufficient length to have unique hybridisation site(s) within the desired template. In general, this means that primer(s) of greater than 10 nucleotides are required for reasonably complex templates. As all possible combinations of a DNA sequence of the length N is given by 4 to the power of N (4^{N}) the number of possible oligonucleotides of sufficient length to allow specificity is very large. The typical length of a primer used for DNA sequencing or amplification is about 15 nucleotides. All possible DNA sequences containing 15 nucleotides could be represented by a library of 4 to the power of 15 (4¹⁵) different oligonucleotides (or over 100 million).

Practical use of oligonucleotides for most applications requires custom chemical synthesis of each oligonucleotide. While many advances have been made in recent years in the automation of oligonucleotide synthesis, this process is still relatively slow and wasteful. For example, limitations in the ability to scale oligonucleotide chemistry often lead to the synthesis of a thousandfold excess of each required primers. This is especially wasteful in applications like primer walking DNA sequencing where each primer might be used for one experiment only (Strauss et al, 1986).

These limitations have led to the development of alternative approaches that utilise pre-synthesised oligonucleotide libraries (Jones & Hardin, 1998). While avoiding the waste and time of custom oligonucleotide synthesis, the use of oligonucleotide libraries is complicated by the large size of useful libraries. For example, even restricting the length of the oligonucleotides to 10 or 11 positions stills results in complete libraries of over a million individual oligonucleotides.

The size of the primer libraries may be reduced by limiting the length of the oligonucleotides (eg. the size of complete libraries of 5-mers and 6-mers are 1024 and 4096, respectively). However, the specificity of such short oligonucleotides is limited. In addition, the requirement for thermostable polymerases in many amplification and sequencing techniques and the consequent demand for high temperatures during the extension procedure, make the use of such short oligonucleotides impracticable.

Other approaches have attempted to utilise partial oligonucleotide libraries of 8 or 9 nucleotides in length (Kieleczawa et al. 1992, Slightom et al. 1994, Jones et al. 1998). However, they have achieved little practical success due to both the large size of such libraries and the inferior hybridisation specificity displayed by oligonucleotides of less then 10 nucleotides.

It is an object of the present invention, therefore, to overcome or ameliorate one or more of the deficiencies of the prior art, or to provide a useful alternative.

### SUMMARY OF THE INVENTION

It has surprisingly been found that oligonucleotides of a required sequence can be synthesised from shorter oligonucleotides thus increasing the affinity of the oligonucleotide for a target nucleic acid and decreasing the number of olignucleotides required in a library of oligonucleotides. The oligonucleotides so synthesised can be used in any application requiring the use of oligonucleotides including, for example, the polymerase chain reaction (PCR), the ligation chain reaction (LCR), reverse-transcriptase PCR (RT-PCR), primer extension reaction for mRNA-transcript analysis, self-sustaining sequence replication, rolling circle amplification, strand displacement amplification, isothermal DNA amplification, DNA-sequencing according to the methods of Sanger (Sanger et al. 1977) or DNA cycle sequencing. The method is particularly suited for use in large-scale amplification or sequencing operations.

The method is based on the hybridisation of two complementary oligonucleotides (an extendable oligonucleotide, “EO”, and a template oligonucleotide, “TO”) and extension of the EO by the addition of bases complementary to the TO.

According to a first aspect, the present invention provides a method of increasing the affinity of an extendable oligonucleotide (EO) for a target nucleic acid comprising:
(a) hybridisation of the EO to a template oligonucleotide (TO) via a region of complementarity, wherein the 5′ region of the TO
   (i) overhangs the 3′ end of the RO; and
   (ii) bears homology to the target nucleic acid; and
(b) extension of the EO such that at least one nucleotide complementary to the TO is added to the 3′ end of the EO, resulting in an extended EO.

Preferably, the EO is of equal or shorter length than the TO. In light of the disclosure provided herewith and the common general knowledge in the field, the skilled addressee will be able to determine the most suitable length of the EO and TO for the particular application required.

The EO and TO may comprise any suitable nucleotides. In a preferred embodiment, they are DNAs although it will be clear to the skilled addressee that other nucleotides and analogues, derivatives or mimics thereof are also contemplated.

The 5′ end of the TO which overhangs the 3′ end of the EO may be of any suitable length from one nucleotide upwards and will be determined by the skilled addressee based on the requirements for the extended EOs as well as other considerations, such as, for example, in large-scale commercial applications, cost and storage capabilities.

Preferably, extension of the EO is achieved by a polymerase. More preferably, the polymerase is E. coli DNA polymerase I, the Klenow fragment of E. coli DNA polymerase, Vent DNA polymerase, Vent (exo⁻), Deep Vent, Deep vent (exo⁻), 9.degree. N DNA polymerase, T4 DNA polymerase, T7 DNA polymerase, T7 RNA polymerase, M-MuLV reverse transcriptase, SP6 RNA polymerase or Taq DNA polymerase. Most preferably, the polymerase has no 5′ to 3′ or 3′ to 5′ exonuclease activities. KIenow 3′ to 5′ exonuclease minus (Klenow 3′-5′ exo) is one example of such a polymerase. In embodiments wherein the EO is other than a DNA, a polymerase such as SP6 or T7 RNA polymerase may be used. The skilled addressee will be able to identify a suitable polymerase for the desired application.

In the context of the present invention, the at least one nucleotide can be any suitable nucleotide, analogue, derivative or mimetic thereof or any other suitable agent or molecule including but is not limited to, a deoxyribonucleotide triphosphate (dNTP), a ribonucleotide triphosphate (rNTP), a peptide-nucleic acid (PNA), a locked nucleic acid (LNA), a 2′-O-methyl rNTP, a thiophosphate linkage, an addition to the amines of the bases (e.g. linkers to functional groups such as biotin), a non-standard base (eg. amino-adenine, iso-guanine, iso-cytosine, N-methylformycin, deoxyxanthosine, difluorotoluene), a virtual nucleotide (eg. Clontech products #5300, #5302, #5304, #5306), non-nucleotide components (eg. Clontech product Nos. 5191, 5192, 5235, 5240, 5236, 5238, 5190, 5225, 5227, 5229, 5223, 5224 and 5222) or a combination or variation thereof Accordingly, the extended EOs of the invention may include the above-mentioned types of nucleotides.

Suitable buffer systems and suitable conditions in which to perform the reactions of the present invention are known to those skilled in the art. Examples of suitable buffers and conditions are provided in the standard references such as Sambrook et al (2001) and the skilled addressee will be able to devise further buffers and conditions based on simple trial and error. Typically, conditions influencing the ability of two oligonucleotides to hybridise include sequence complementarity, salt- and solute-concentration, temperature, pH, pressure, oligonucleotide concentration and secondary structure of the nucleic acid itself.

In certain embodiments, the extended EO may be purified from the other components in the reaction mixture (ie. buffer reagents, TO, nucleotides, polymerase etc.). This can be accomplished using standard oligonucleotide separation techniques known to the person skilled in the field (Sambrook et al, 2001). Alternatively, the extended EO may be directly used in a further reaction without purification.

Preferably, the extended EO is dissociated from the TO and used to bind to the target nucleic acid in a further method. Examples of methods in which the extended EOs of the present invention may be used are the polymerase chain reaction (PCR), the ligation chain reaction (LCR), reverse-transcriptase PCR (RT-PCR), primer extension reaction for mRNA-transcript analysis, self-sustaining sequence replication, rolling circle amplification, strand displacement amplification, isothermal DNA amplification, DNA-sequencing according to the methods of Sanger (Sanger et al. 1977) and DNA cycle sequencing. Other methods in which the extended EOs of the present invention may be used will be recognised by the skilled addressee and fall within the scope of this invention.

The skilled addressee will recognise that the 3′ end of the TO may optionally also be extendable by a polymerase.

In the present invention the TO can be extendable or its extension can be blocked. Blockage can be achieved by a TO design that creates a non-hybridising 5′ overhang of the EO, providing no template for the extension of the TO. If a 5′ overhang of the EO is provided, then extension of the TO can be prevented by modification of its 3′ end rendering it unrecognisable or non-extendable by a polymerase. Such modifications include, but are not restricted to, addition of phosphate groups, biotin, carbon-chains, amines, dideoxyribonucleotides or other molecules to the 3′ end or by a 3′ end that is not hybridising to the 5′ region of the EO.

The degree of homology of the 5′ end of the TO to the target nucleic acid may be determined by the skilled addressee and will vary according to the application for which the extended EOs are required.

In certain embodiments the present invention may include the incorporation of degenerate or universal nucleotides into the EO or TO. When TOs include degenerate or universal nucleotides, for example, this allows for one specific TO to hybridise to several different EOs and hence reduces the number of TOs required in a TO library. A degenerate oligonucleotide is effectively a mixture of oligonucleotides in which different nucleotides are included at the degenerate position in the oligonucleotide. For example, an oligonucleotide with the sequence GGTNGC would consist of oligonucleotides with the following sequence: GGTAGC, GGTTGC, GGTGGC and GGTCGC. A universal nucleotide is a nucleotide or nucleotide analogue incorporated into a nucleic acid that has similar or identical hybridisation properties to a number of other nucleotides. Such nucleotides or nucleotide-analogues include, but are not restricted to, inosine, 3-nitropyrrole and 5-nitroindole.

According to a second aspect, the present invention provides a method of amplifying a target nucleic acid comprising
(a) hybridisation of an extendable oligonucleotide (EO), to a template oligonucleotide (TO), wherein the 5′ region of the TO
   (i) overhangs the EO by at least one nucleotide; and
   (ii) bears homology to the target nucleic acid; and
(b) extension of the EO such that at least one nucleotide complementary to the TO is added to the 3′ end of the EO.
(c) amplification of the target nucleic acid utilising the extended EO.

According to a third aspect, the present invention provides a method of sequencing a target nucleic acid comprising
(a) hybridisation of an extendable oligonucleotide (EO) to a template oligonucleotide (TO), wherein the 5′ region of the TO
   (i) overhangs the EO by at least one nucleotide; and
   (ii) bears homology to the target nucleic acid; and
(b) extension of the EO such that at least one nucleotide complementary to the TO is added to the 3′ end of the EO; and
(c) dissociation of the annealed oligonucleotides and utilising the extended EO in a sequencing reaction.

According to a fourth aspect, the present invention provides a pair of oligonucleotides comprising an extendable oligonucleotide (EO) and a template oligonucleotide (TO) wherein
(a) the EO comprises a region complementary to a region of the TO;
(b) the EO is extendable at its 3′ end; and
(c) wherein the 5′ end of the TO is such that if the EO and TO were annealed, the 5′ end of the TO would overhang the 3′ end of the EO by at least one nucleotide.

Preferably, the at least one nucleotide is substantially similar to, or identical with, a nucleotide in a target nucleic acid. The at least one nucleotide may be any number of nucleotides and any one or more of the nucleotides may be substantially similar to, or identical with, the nucleotides of the target nucleic acid. The target nucleic may be a nucleic acid, for example, such as the nucleic acid of any one of the first to third aspects.

According to a fifth aspect, the present invention provides a library comprising a plurality of pairs of oligonucleotides according to the fourth aspect.

According to a sixth aspect, the present invention provides two complementary libraries, one comprising EOs and the other comprising TOs wherein the EOs and TOs are suitable for use in a method according to any one of the first to third aspects.

According to the seventh aspect, the present invention provides a library comprising a plurality of oligonucleotides with a common constant region and a variable region specific for each member of the library.

According to an eighth aspect, the present invention provides a kit comprising a library of extendable oligonucleotides (EOs) and a complementary library of template oligonucleotides (TOs) wherein
(a) the EOs comprise a region complementary to a region of the TOs;
(b) the EO is extendable at its 3′ end; and
(c) wherein the 5′ end of the TOs is such that when an EO from the library of EOs and a TO from the library of TOs are annealed, the 5′ end of the TO overhangs the 3′ end of the EO by at least one nucleotide.

The complementary region, or part of the complementary region, of the EO and TO may be termed a “clamp”. It will be clear to the skilled addressee that the EOs and TOs may contain more than one region of complementarity.

The clamp region generally provides stability for hybridisation of the EO and the TO under conditions where the extension of the EO can take place. In one or more embodiments, the clamp region may contain sequence motifs useful for subsequent applications, such as recognition sequences for restriction endonucleases, phage polymerase transcription signals, binding sites for ribosomes, or start codons enabling translation. In a preferred embodiment, the clamp region is a region that is fully complementary between the EO and TO i.e. for every base in the clamp region of the EO there is a complementary base in the TO.

Preferably, the complementary regions of the EO and TO comprise sequence motifs. These motifs when included in the clamp region can provide stringent hybridisation of the EO and TO which may increase the efficiency of extension. Such motifs are known to those skilled in the art and frequently contain a high G+C content. In addition, the sequence of the clamp region should preferably contain little sequence similarity to known common motifs or sequence of the template. For example, if the target is a DNA insert within a plasmid or cosmid then a clamp design with little complementarity to the plasmid or cosmid backbone sequence will ensure that the unextended or extended EO will not hybridise to unspecific sites on the plasmid backbone.

In one or more embodiments, the 3′ region of the EO is variable and, as such, in the context of the present invention the term “the EO” may include a mixture of EOs comprising a number of different oligonucleotides.

Similarly, the 5′ region of the TO may be variable.

In one or more embodiments, the TO may include a catch region. The catch region comprises one or more degenerate or universal nucleotides. It may lie between a constant 3′ region of the TO and a variable region and it may be adjacent to, or form part of, the clamp region. Due to its degenerate or universal positions the catch region may hybridise in all or most of its positions with many or all members of the EO library. This will allow for the polymerase-mediated extension of many or all of the members of a complementary EO library after hybridisation with the members of the TO library. The design of a typical EO and TO library is illustrated in FIG. 2.

The skilled addressee will understand that since G/C pairs form stronger interactions than A/T pairs, it is preferable that the nucleotides closest to the 3′ end of the EO are G or C and that the TO comprises G or C (as appropriate) in the complementary positions. In this way, the EO and TO are likely to anneal more tightly providing a better template for extension by, for example, a polymerase.

One skilled in the art will recognise that the size of the libraries is determined by the number of variable positions. For example, if the variable region of the EO library that hybridises with the catch region of the TO library is 5 positions long, then there would be 1024 (4⁵) possible members of the EO library. Similarly, if the TO library has 5 positions which serve as template for extension, then a complete library would also contain 1024 members. It is also apparent that the complete library of extended EO primers is dependent on the size of the TO library, that is the number of possible templates in the TO library determines how many different extension products can be made from each member of the EO library. Using the previous example, the number of all possible extended EOs would be 1024×1024=1048576 (4¹⁰).

The present invention also includes libraries with oligonucleotides having either different clamp structure or sequence, different designs of the catch region or different lengths or compositions of the variable regions.

In a preferred embodiment the EO and TO comprise the following nucleotides:

EO: 5′ YYYYYXXXXX
|||||
TO: 3′ YYYYYNNNNNXXXXX

wherein the Y nucleotides are complementary, fixed nucleotides, and N, S and X are as herein defined. More preferably, the sequence of the TO in this preferred embodiment is 3′ YYYYYNNNSSXXX 5′.

According to a ninth aspect, the present invention provides a kit comprising a pair of oligonucleotides according to the fourth aspect, or a library or libraries of oligonucleotides according to any one of the fifth to eighth aspects.

Definitions/Abbreviations

Generally, the terminology and abbreviations used throughout the specification are standard and will be familiar to those skilled in the art or have been explained in the text. In the interests of clarity, however, a number of definitions have been supplied below.

With respect to the examples included in the description of the present invention, the following standard abbreviations for nucleotides have been used: “A” represents adenine as well as its deoxyribonucleotide derivatives. “T” represents thymine as well as its deoxyribonucleotide derivatives, “G” represents guanine as well as its deoxyribonucleotide derivatives, “C” represents cytosine as well as its deoxyribonucleotide derivatives. “N” represents A, T, C or G. Generally, N is used to indicate that in a mixture of DNAs, the mixture contains at least four types of DNAs which have, alternatively, an A, T, C or G at the N position. “X” represents an unknown nucleotide and may be A, T, C or G. In contrast to N, X is not generally used when referring to a mixture of DNAs, rather it generally represents a fixed but unknown nucleotide eg. an unknown nucleotide in a genomic DNA molecule. “S” represents G or C. “I” represents inosine.

In the context of the present invention, the term “complementary” refers to the relationship between two nucleotides or oligonucleotides/polynucleotides. In the context of DNA, generally A is complementary to T and G is complementary to C. As such, when two DNAs (eg. oligonucleotides) align, A on one DNA will generally bind to T on the other DNA and G on one DNA will generally bind to C on the other DNA. When such binding occurs, the DNAs (eg. oligonucleotides) are annealed or hybridised.

In the context of the present invention, the terms “annealing”, “anneals”, “hybridises”, “hybridising”, “hybridisation” and derivatives thereof refer to the process whereby two single-stranded DNAs form a double-stranded molecule. Usually this involves the DNAs forming hydrogen bonds between at least some of the complementary nucleotides of the two strands i.e. the formation of G/C and/or A/T pairs.

Hybridisation of two DNAs (eg oligonucleotides) is dependent on a number of factors, including the degree of complementarity of their respective sequences, the concentration of the DNAs, the surrounding temperature and/or pressure, or the prevailing chemical conditions/composition of the environment such as ionic strength, pH and the presence of denaturing agents (eg. formaldehyde, urea, formamide). For example, the strength of the binding between two oligonucleotides generally increases with increased sequence complementarity, higher DNA concentration, lower temperature, increased pressure, higher ionic strength and lower concentration of denaturing agents.

In the context of the present invention, the term “DNA molecule” refers to a single-stranded or double-stranded deoxyribonucleotide comprised of a polymer made from the bases A, T, C and G or variations thereof.

In the context of the present invention, the term “polymerase” refers to an enzyme which catalyses the synthesis of polynucleotides eg. DNA, oligonucleotides. The polymerase used in nucleic acid amplifications and cycle sequencing reactions is typically a heat-stable enzyme that allows for heat denaturation of the template without degradation of the polymerase. The polymerase can generate a new strand from an oligonucleotide (“primer”) hybridised to the template. Since the primer is extended at elevated temperature, secondary structures that could otherwise interfere with the extension are minimised. The polymerase then includes in the polynucleotide strand being synthesised (in the 5′ to 3′ direction), nucleotides or derivatives thereof complementary to those of the template strand.

In the context of the present invention, the term “extension product” refers to the nucleic acid synthesised from the 3′ end of a primer which nucleic acid is complementary to the strand of DNA to which the primer is hybridised.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### BRIEF DESCRIPTION OF THE FIGURES

FIG. 1. Schematic presentation of the hybridisation of different extendable oligonucleotides (EOs) and template oligonucleotides (TOs). Vertical bars indicate hybridisation of complementary nucleotide regions.

FIG. 2. Schematic representation of the design for an EO and TO library with “clamp” and “catch” regions. X represents a specific nucleotide that is different for each member of the library. N represents a degenerate position that may be G, C, T or A—the library contains oligonucleotides each having one of the possible nucleotides in the N position. Vertical bars indicate hybridisation of complementary nucleotides.

FIG. 3. Partial human genomic DNA region of the p53 gene. The underlined section represents the direct binding site of the extended EOs and arrows indicate the direction of extension. The sequence is given 5′ to 3′ for the coding DNA strand.

FIG. 4. Design of EOs and TOs for the amplification of a region of the human p53 gene. Horizontal bars indicate regions of hybridisation between the EO and TO. Capital letters show actual sequence of the oligonucleotides and small, underlined letters indicate the extended region of the EO. F and R stand for the oligonucleotide pairs targeting the forward and reverse region, respectively, as shown in FIG. 3.

FIG. 5. Amplification of a 1625 bp region of the human p53 gene. Lane 1 contains an amplification reaction with oligonucleotides EOp53F, TOp53F, EOp53R and TOp53R. Lane 2 contains an amplification reaction with oligonucleotides EOp53F and EOp53R (negative control). Lane 2 contains an amplification reaction with Cp53F and Cp53R (positive control). Five microlitres of each reaction were separated on a 1% (w/v) agarose gel and stained with ethidium bromide. Lane 4 contains one microlitre of a 1 kb⁺ ladder marker (Life Technologies, Rockville, Mass., USA) with some DNA-sizes (bp=basepairs) shown on the right.

FIG. 6. DNA sequence of Escherichia coli ftsZ and the recognition sequence for the M13 reverse primer (double underlined) within the plasmid pFC1. Dotted and solid underlining indicate the target region for the EOs. For further details see the text.

FIG. 7. Design of EO and TO for the amplification of a region of Escherichia coli ftsZ gene. Horizontal bars indicate clamp regions of hybridisation between the EO and TO. Capital letters show actual sequence of the oligonucleotides and small, underlined letters indicate the extended region of the EO. N represents a position with either A, T, G or C. S represents a position with either G or C nucleotide.

FIG. 8. Amplification of the E. coli ftsZ gene. Lane 1 contains one microlitre of a 1 kb⁺ ladder marker (Life Technologies) with some DNA-sizes (bp=basepairs) shown on the left. Lane 2 contains an amplification reaction with oligonucleotides EC10 and M13 reverse (positive control). Lane 3 contains an amplification reaction with oligonucleotides E128, T128 and M13 reverse. Lane 4 contains an amplification reaction with oligonucleotides E128 and M13 reverse (negative control). Lane 5 contains an amplification reaction with oligonucleotides E382, T382 and M13 reverse. Lane 6 contains an amplification reaction with oligonucleotides E382 and M13 reverse (negative control). Five microlitres of each reaction were separated on a 1% (w/v) agarose gel and stained with ethidium bromide.

FIG. 9. Electropherogram of a DNA sequencing reaction with the extended EO128 and a linear DNA template. The sequencing reaction was separated and analysed on an ABI PRISM™ 377 DNA sequencer and ABI PRISM™ sequence analysis software.

FIG. 10. Electropherogram of a DNA sequencing reaction with an incorporated EO/TO hybridisation and extension. The sequencing reaction was separated and analysed on an ABI PRISM™ 377 DNA sequencer and ABI PRISM™ sequence analysis software.

FIG. 11. Design of EO and TO libraries. Y represents a specific nucleotide of the “clamp” region, while X represents a nucleotide specific for each member of the library. N represents a degenerate position with either A, T, G or C. S represents a degenerate position with either G or C.

FIG. 12. DNA sequence of the partial Escherichia coli streptomycin operon and the target sequence for an extended EO (underlined).

FIG. 13. Design of an EO and an TO for the sequencing of a region of the Escherichia coli streptomycin operon. Horizontal bars indicate regions of clamp region of hybridisation between the EO and TO. Capital letters show actual sequence of the oligonucleotides and small, underlined letters indicate the extended region of the EO. N stands for a position with either A, T, G or C and S stands for a position with either G or C nucleotide.

FIG. 14. Electropherogram of a DNA sequencing reaction using EO827 and TO827N3. The sequencing reaction was separated and analysed on an ABI PRISM™ 377 DNA sequencer and ABI PRISM™ sequence analysis software.

FIG. 15. DNA sequence of the partial Escherichia coli streptomycin operon and the target sequence for an extended EO (indicated by lines). Target regions for extended EO primers are numbered and referred to in the text.

FIG. 16. Design of an EO and an TO for the sequencing of a region of the Escherichia coli streptomycin operon. Horizontal bars indicate regions of clamp region of hybridisation between the EO and TO. Capital letters show actual sequence of the oligonucleotides and small, underlined letters indicate the extended region of the EO. N stands for a position with either A, T, G or C and S stands for a position with either G or C nucleotide.

FIG. 17. Design of EOs and TOs for the sequencing of a region of the Escherichia coli streptomycin operon. References to target sites in FIG. 15 are given. Horizontal bars indicate regions of clamp region of hybridisation between the EO and TO. Capital letters show actual sequence of the oligonucleotides and small, underlined letters indicate the extended region of the EO. N stands for a position with either A, T, G or C.

FIG. 18. Design of TO primers for the extension of E827 and for the sequencing of a region of the Escherichia coli streptomycin operon. Horizontal bars indicate regions of clamp region of hybridisation between the EO and TO. Capital letters show actual sequence of the oligonucleotides and small, underlined letters indicate the extended region of the EO. N stands for a position with either A, T, G or C and S stands for a position with either G or C nucleotide.

FIG. 19. Region of the E. coli genome sequence targeted for amplification. The target sequences (5′ to 3′) for the extended EO primers are underlined.

FIG. 20. Design of EO and TO primers for the amplification of a region of the E. coli genome. Horizontal bars indicate regions of hybridisation between the EO and TO. Capital letters show actual sequence of the oligonucleotides and small, underlined letters indicate the extended region of the EO. F and R stand for the oligonucleotide pairs targeting the forward and reverse region, respectively, as shown in FIG. 19.

FIG. 21. Amplification of a 211 bp genomic DNA region from E. coli using extendable and template oligonucleotides. The desired extension product is indicated by a white arrow. Lane 1 contains a marker with size (in basepairs) given on the left. Lane 2 contains the amplification reaction with Klenow-treatments containing EOF, TOF, EOR and TOR. Lane 3 contains the same reaction as Lane 2 but with omission of TOF and TOR. Lane 4 contains the same reaction as Lane 2 but with omission of EOF and EOR. Further details are given in the text.

FIG. 22. DNA-sequence of pUC19 plasmid. The regions of binding for extended EO (indicated as EO/TO pairs) are shown underlined.

FIG. 23. Electropherogram of a DNA sequencing reaction using E154 and T422. The sequencing reaction was separated and analysed on an ABI PRISM™ 377 DNA sequencer and ABI PRISM™ sequence analysis software.

FIG. 24. Electropherogram of a DNA sequencing reaction using E167 and T14. The sequencing reaction was separated and analysed on an ABI PRISM™ 377 DNA sequencer and ABI PRISM™ sequence analysis software.

### DESCRIPTION OF THE INVENTION

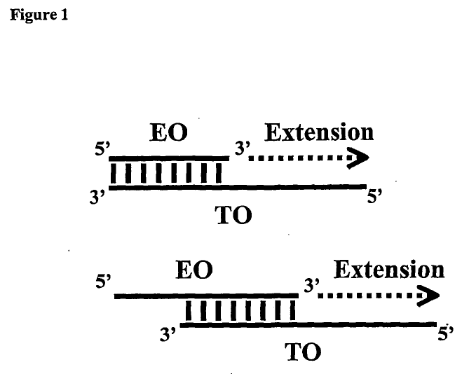

The present invention provides a method for the production of oligonucleotides by the hybridisation of two complementary oligonucleotides (an extendable oligonucleotide, “EO”, and a template oligonucleotide, “TO”) and extension of the EO by the addition of bases complementary to the TO. Examples of such EOs and TOs are shown in FIG. 1. The oligonucleotides are suitable for use in any method in which oligonucleotides are required and especially in methods such as the polymerase chain reaction (PCR), the ligation chain reaction (LCR), reverse-transcriptase PCR (RT-PCR), primer extension reaction for mRNA-transcript analysis, self-sustaining sequence replication, rolling circle amplification, strand displacement amplification, isothermal DNA amplification, DNA-sequencing according to the methods of Sanger (Sanger et al. 1977) and DNA cycle sequencing.

In accordance with the method of the invention, an oligonucleotide primer having 5′ and 3′ ends is incubated with a relatively longer oligonucleotide template having a 5′ region non-complementary to the primer and a 3′ region complementary to the primer. The annealed product is reacted with at least one nucleotide in the presence of a template-dependent polynucleotide polymerase to produce a primer extended at its 3′ end by at least one nucleotide complementary to the 5′ region of the template. This primer can be used for any method currently employing oligonucleotide primers as mentioned above.

Upon completion of the reaction, the EO is increased in length and the additional nucleotides included in the EO are determined by the non-hybridising 5′ region of the TO. The extended EO may thus hybridise to a template under conditions where the non-extended EO might fail to hybridise. Conditions influencing the ability of two oligonucleotides to hybridise include sequence complementarity, salt- and solute-concentration, temperature, pH, pressure, oligonucleotide concentration and secondary structure of the oligonucleotide itself.

In the present invention the TO can be extended or its extension can be blocked. This can be achieved by a TO design that creates a non-hybridising 5′ overhang of the EO, essentially providing no template for the extension of the TO. If a 5′ overhang of the EO is present, then extension of the TO can be prevented by modification of its 3′ end rendering it unrecognisable or non-extendable by a polymerase. Such modifications include, but are not restricted to, addition of phosphate groups, biotin, carbon-chains, amines, dideoxyribonucleotides or other molecules to the 3′ end or by a 3′ end that is not hybridising to the 5′ region of the EO.

The present invention may also include the incorporation of degenerate or universal nucleotides into the EO or TO. Inclusion of degenerate or universal nucleotides in the TO, for example, allows for one specific TO to hybridise to several different EOs and hence reduces the number of TOs required in a library. A degenerate oligonucleotide is effectively a mixture of oligonucleotides in which different nucleotides are included at the degenerate position in the oligonucleotide. For example, an oligonucleotide with the sequence GGTNGC would consist of oligonucleotides with the following sequence: GGTAGC, GGTTGC, GGTGGC and GGTCGC. A universal nucleotide is a nucleotide or nucleotide analogue incorporated into a nucleic acid that has similar or identical hybridisation properties to a number of other nucleotides. Such nucleotides or nucleotide-analogues include, but are not restricted to, inosine, 3-nitropyrrole and 5-nitroindole.

Template libraries and kits containing these libraries for use in conjunction with the polynucleotide synthesis method can also be prepared. The present invention provides a method to generate a library of primers with sufficient complexity and hybridisation specificity to enable practicable amplification or sequencing of most DNA templates. For example, using the present invention every possible oligonucleotide with a length of greater than 10 can be produced by the combination and enzymatic treatment of two specific oligonucleotides selected from two libraries of relatively small size. The production of the specific larger oligonucleotide can be performed prior to the application of the primer, or be directly incorporated into the DNA-amplification or sequencing procedure.

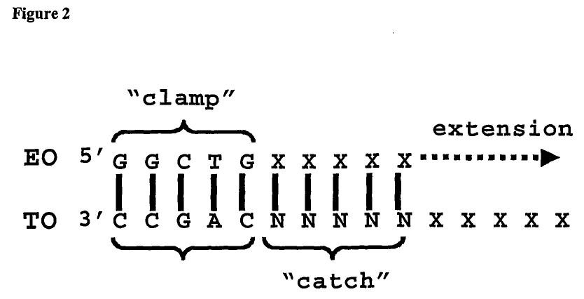

The design of a typical EO and TO library scheme is illustrated in FIG. 2.

Preferred embodiments of the invention will now be described, by way of example only, with reference to the accompanying Figures.

### EXAMPLE 1

### Amplification of the Human Gene for the Protein P53 Using Two Extendable Oligonucleotides and Two Template Oligonucleotides

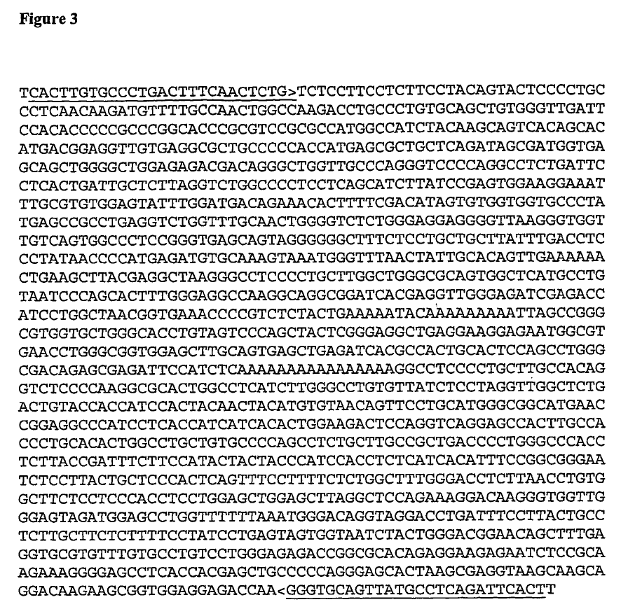

This example produces two oligonucleotides by the hybridisation and extension of two EO/TO pairs. It is shown that the extended EO has improved affinity for the target nucleic acid in a subsequent application when compared to the unextended EO. In this example, the extended BO is used without any further treatment in a reaction amplifying a 1625 base pair region of the human p53 gene. The region of human genomic DNA targeted by the amplification in this experiment is given in FIG. 3.

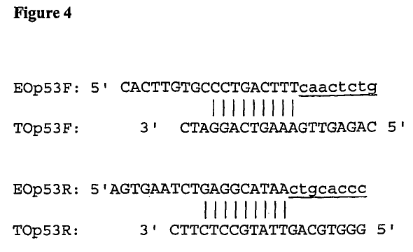

Based on the target sequence in the p53 gene the following primers were designed and FIG. 4 shows the hybridisation and the extension of these oligonucleotides.

In FIG. 4, the final three 3′ terminal nucleotides of the two TO primers do not hybridise with their respective EO templates and are therefore not extendable by a template-dependent polymerase lacking 3′ to 5′ exonuclease activity. The EO primers are designed to hybridise their respective target sequence over 18 nucleotides, resulting in a moderately strong binding. This binding is illustrated through a predicted annealing temperature of 57.5° C. for EOp53F or EOp53R as calculated by the nearest-neighbour method (Santa Lucia, 1988). In contrast, the extended oligonucleotides have a greater length (8 nucleotides more) and a longer region of complementarity with the target sequence (26 nucleotide positions). As a result hybridisation affinity for the target nucleic acid is increased and the target is more stable as indicated by a predicted annealing temperature of the extended EOp53F or EOp53R of 72.9° C. as calculated by the nearest-neighbour method.

The initial hybridisation and extension of the EO primers was performed in a single reaction containing the following reagents (in a final volume of 10 microlitres): 1.25 micromolar EOp53F, 1.25 micromolar EOp53R, 10 micromolar TOp53F, 10 micromolar TOp53R, 2.5 millimolar dNTPs (MBI Fermentas, Vilinius, Lithuania), 10 millimolar 50 tris(hydroxymethyl) aminomethyl hydrochloride (Tris-HCl) (pH 8.5 at 25° C.), 5 millimolar magnesium chloride (MgCl₂), 1 millimolar dithiothreitol, 1 unit of Klenow Exo⁻ (MBI Fermentas, Vilinius, Lithuania). For the negative control reaction TOp53F and TOp53R were omitted. For the positive control EOp53F, EOp53R, TOp53F and TOp53R were omitted and instead the control primers Cp53F (5′-CACTTGTGCCCTGACTTTCAACTCTG-3′) and Cp53R (5′-AGTGAATCTGAGGCATAACTGCACCC-3′) were added at 1.25 micromolar each. The reactions were incubated at room temperature (21° C.) for 30 min and at 70° C. for 10 min.

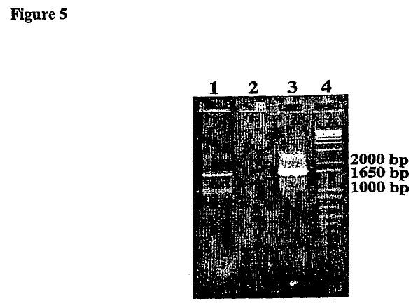

For amplification, the entire reaction from above was added to 3 microlitres of 25 millimolar MgCl₂, 5 microlitres of 10×PCR buffer [100 millimolar Tris-HCl (pH 9 at 25° C.), 500 millimolar potassium chloride (KCl), 1% (v/v) Triton X-100 (Promega, Madison Wis., USA)], 2.5 microlitres human genomic DNA (300 nanograms per microlitre), and water to a final volume of 50 microlitres. The reactions were heated for 10 min at 95° C. and then 0.5 microlitres of a Taq/Pfu polymerase mix (unit ratio of 10:1; unit concentration 0.25 unit per microlitre; Promega) was added. The reaction was then cycled 32 times at 95° C. for 30 sec, at 70° C. for 30 sec and at 72° C. for 2.5 min. After a final heating step of 7 min at 72° C. the reaction was then stored at 4° C. Ten microlitres of the reactions were then separated on a 1% (w/v) agarose gel and stained with ethidium bromide using standard techniques (Sambrook et al. 1989.). FIG. 5 shows the result of this experiment.

The reaction containing EOp53F, TOp53F, EOp53R and TOp53R shows a band at around 1650 bp (FIG. 5, lane 1) corresponding to the correct amplification product of the p53 gene (positive control is shown in FIG. 5, lane 3). The production of the correct amplicon is dependent on the presence of the TO primers as no band is visible for the reaction containing only the EO primers (negative control; FIG. 5, lane 2). This demonstrates that a TO-dependent extension of EO must occur in order for the EO to function in the specific amplification reaction. The negative control reaction in FIG. 5 (lane 2) illustrates that the non-extended EOs are not suitable for the subsequent application of specific DNA amplification.

### EXAMPLE 2

### Amplification of a DNA Region Using One Extendable Oligonucleotide, One Template Oligonucleotide with Degenerate Positions and One Specific Oligonucleotide

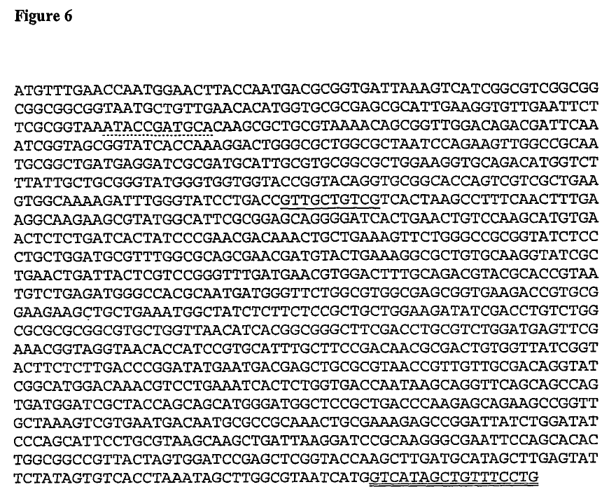

This example demonstrates the hybridisation and extension of an EO directly within a DNA-amplification reaction. In addition the hybridisation and extension of an EO with a TO containing a degenerate catch region and a clamp region is shown. The target for the amplification reaction is a 4.6 kilobase pairs plasmid (PFC1) containing the ftsZ gene from Escherichia coli and a region complementary to the specific primer M13 reverse. The sequence of the linear DNA fragment and the target region is shown in FIG. 6.

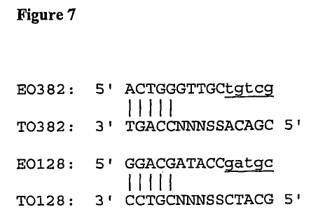

Two EO/TO primer pairs were designed to target different regions on the plasmid template. The first pair (EO 382/TO 382; FIG. 7) is targeted to the sequence 5′-GTTGCTGTCG-3′ (underlined positions shown in FIG. 6) and the second pair (EO 128/TO128; FIG. 7) is directed towards the sequence 5′ATACCGATGCA 3′ (see dotted region in FIG. 6).

One important difference between this oligonucleotide design here and the one used in the Example 1 is that a catch region with degenerate positions is incorporated in the TO primers. The 3′ end of the catch region contains two positions with restricted degeneracy (only G and C). This allows for efficient hybridisation of the two 3′ terminal nucleotides of the EO, since 25% of the TO molecules have the complementary sequence for these two positions. A perfect match of the 3′ end of the EO primers may be necessary for efficient extension by a template-dependent polymerase. As only the 5 nucleotide positions of the 3′ side of the non-extended EO primer can hybridise to the target sequence of the linear DNA, they are not able to be extended except under very non-stringent conditions. After polymerase extension, 10 positions of the EOs are now complementary to the target sequence. This results in an increase in the hybridisation efficiency under stringent conditions. In this example Taq-polymerase is used to extend the EOs. Taq DNA polymerase can add a non-template dependent adenosine-residue at the 3′ end of an extension product. The efficiency of the addition depends in a complex fashion on the 5′ sequence of the template (Brownstein et al. 1996, Magnuson, et al. 1996). This fact was considered in the design of E128 whereby an additional non-template A at the 3′ end of the EO hybridises with a complementary T nucleotide in the target sequence (FIG. 6). In contrast, an additional A-nucleotide added to the E382 will not hybridise with the target template, thus creating a potentially non-extendable 3′ end.

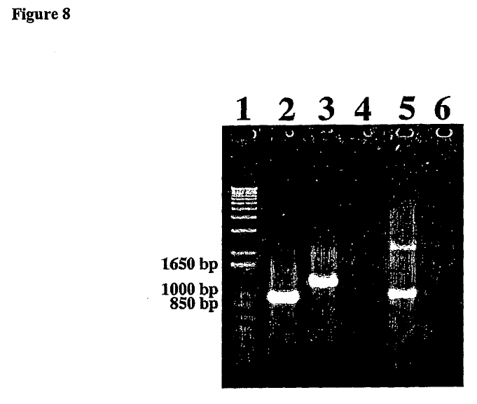

For hybridisation and extension of EO and the amplification reaction of the target template the following reagents were combined: one microlitre of pFC1 plasmid (1 nanogram per microlitre), one microlitre of the EO (10 picomoles/microlitre), one microlitre of the TO (20 picomoles/microlitre), one microlitre of M13 reverse primer (5′-CAGGAAACAGCTATGAC-3′; 5 picomoles/microlitre), two microlitres of 25 millimolar MgCl₂, four microlitres of 1 millimolar dNTPs (MBI Fermentas, Vilinius, Lithuania), two microlitres of 10× buffer [100 millimolar Tris-HCl (pH 9 at 25° C.), 500 millimolar potassium chloride (KCl), 1% (v/v) Triton X-100 (Promega)], and water to final volume of 16 microlitres. For the negative control reaction the TO primer was omitted. For the positive control the EO and TO were omitted and the control primer EC10 (5′GTTGCTGTCG 3′) targeting the same region as the E382/TO382 pair was added (one microlitre of a 10 picomoles/microlitre solution). The mixture was heated for one min at 95° C. and then cooled to 80° C. at which stage four microlitres of Taq DNA polymerase (0.25 units/microlitre; Promega) was added. The reactions were then cycled 32 times at 95° C. for 10 sec, at 51° C. for 20 sec and at 72° C. for 1.5 min. After a final heating step for 5 min at 72° C. the reactions were stored at 4° C. Five microlitres of the reaction were then separated on a 1% (w/v) agarose gel and stained with ethidium bromide using standard techniques (Sambrook et al. 1989.). FIG. 8 shows the result of this experiment.

The reaction containing EO128, TO128 and M13 reverse primers shows a band of approximately 1150 base pairs (FIG. 8, lane 4) which correlates with the predicted size of 1165 base pairs (see FIG. 6). The production of the amplicon is dependent on the presence of TOs as no band is visible for the reaction containing only EO128 and M13 reverse (negative control; FIG. 8, lane 2). This demonstrates that a TO-dependent extension of EO must occur in order for the EOs to function in an amplification reaction. The intensity of the EO amplicon band is almost as strong as for the positive control containing BO10C and M13 reverse. The reaction containing EO382, TO382 and M13 reverse shows a band at around 900 bp (see FIG. 8 lane 5) correlating well with the predicted size of 911 base pairs (FIG. 6). The production of the correct amplicon is dependent on the presence of the TO primer as no band is visible for the reaction containing only EO382 and M13 reverse (negative control; FIG. 8, lane 6). The intensity of the amplicon band is slightly weaker than the positive control and the reaction containing EO128, TO128 and M13 reverse. This is possibly due to a 3′ terminal A overhang added by the Taq DNA polymerase to some extended EO382 molecules. These A overhang molecules will not be extended during the exponential template amplification, thus somewhat reducing the efficiency of the reaction. It was also noted in this experiment, that the EO128/TO128 design appears to provide greater specificity than the EO382/TO382 pair. Without being bound by theory, this may be due to the effect that an extended EO382 with the addition of a 3′ A has the specificity of an 11-mer while the suitably extended EO382 is a 10-mer.

The ratio between EO to TO of 1:2 in this experiment has also been varied. Similar amplification results were obtained for ratios between 1: 0.5 and 1:2 for the EO to TO ratio. Higher EO to TO ratios caused weaker product formation, while lower ratios caused the appearance of unspecific amplification products (data not shown).

### EXAMPLE 3

### Cycle Sequencing of DNA Using One Extendable and One Template Oligonucleotides with Previous Enzymatic Treatment

This example shows the application of an extended EO in a DNA sequencing reaction.

E128 (Example 2) was extended in a reaction containing in a final volume of 10 microlitres the following reagents: 10 micromolar E128, 40 micromolar T128, 1 millimolar dNTPs (MBI Fermentas, Vilinius, Lithuania), 10 millimolar 50 tris(hydroxymethyl)aminomethyl hydrochloride (Tris-HCl) (pH 8.5 at 25° C.), 5 millimolar magnesium chloride (MgCl₂), 1 millimolar dithiothreitol, and 1 unit of Klenow Exo⁻ (MBI Fermentas, Vilinius, Lithuania). The reactions were incubated at room temperature for 30 min. One unit of shrimp alkaline phosphatase (Roche, Basel, Switzerland) was added to the reaction and incubated for 30 min at 37° C. and 20 min at 65° C. This step was applied to remove excess dNTPs from the extension reaction which would potentially interfere with the subsequent sequencing reaction.

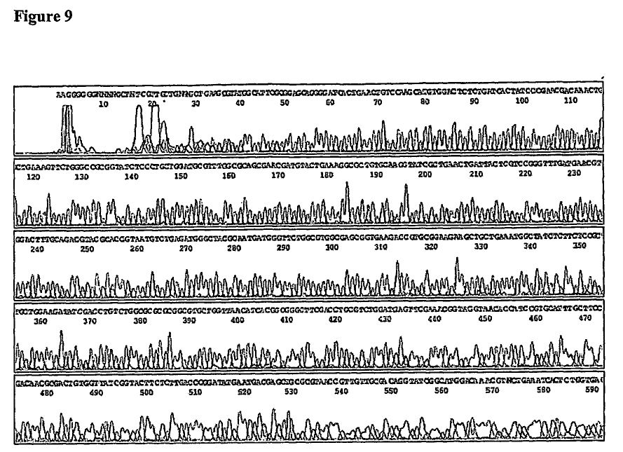

For DNA sequencing a 1.3 kb linear DNA fragment containing the entire ftsZ gene from E. coli as used. The sequencing reaction contained the following reagent (final volume of 8 microlitres): 3 microlitres BigDye™ sequencing reagent (Applera Corporation, Norwalk, Conn. USA), 1 microlitre (100 nanograms) linear DNA template, 1 microlitre of the B128 extension reaction and 3 microlitres of water. The reaction was then cycled 40 times at 96° C. for 10 sec, at 45° C. for 30 sec and at 60° C. for 4 min. The sequencing reaction was purified as described by Tillett and Neilan (1999). The cleaned sequencing reaction was analysed on an ABI PRISM™ 377 DNA sequencer using the ABI PRISM™ sequence analysis software (Applera Corp., Norwalk, Conn., USA) according to the manufacturer's instructions. FIG. 9 shows the resulting sequence electropherogram of the experiment.

Good signal intensity and the correct sequence (compare to FIG. 6) was obtained. A negative control in which the TO128 was omitted in the initial extension reaction and all subsequent steps kept the same, yielded no sequence data (data not shown). This shows that the sequencing success above is dependent on a TO128-dependent extension of EO128.

### EXAMPLE 4

### Incorporation of Oligonucleotide Extension into Cycle Sequencing of DNA Using One Extendable and One Template Oligonucleotide

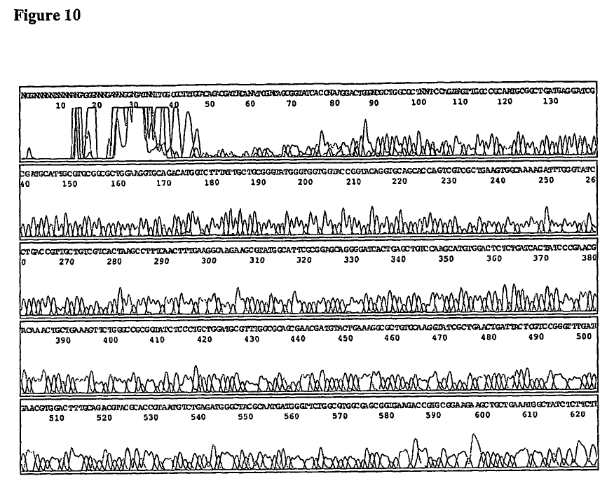

This example demonstrates the direct incorporation of the EO/TO hybridisation and extension method into a DNA-cycle sequencing protocol in a single reaction. For the sequencing reaction the BigDye™ sequencing system (Applera Corporation, Norwalk, Conn. USA) was used. This system was supplemented with Klenow Exo⁻ polymerase, magnesium chloride, dithiothreitol (DTT) and dGTP to ensure optimal extension of the EO. The sequencing reagents contained the following components (in a final volume of 10 microlitres): One micromolar EO128, 4 micromolar TO128,100 nanograms linear template DNA (see Example 3), 1.25 millimolar MgCl₂, 1 millimolar DTT, 0.1 unit Klenow Exo⁻ (MBI Fernentas, Vilinius, Lithuania), 20 micromolar dGTP, and 4 microlitres of BigDye™ sequencing reagent. The mixture was incubated at room temperature (23° C.) for 30 min and cycled 40 times at 96° C. for 10 sec, at 45° C. for 30 sec and at 60° C. for 4 min. The reaction was purified as described in Example 3. The sequencing reaction was analysed on an ABI PRISM™ 377 DNA sequencer and ABI PRISM™ sequence analysis software (Applera Corp., Norwalk, Conn., USA) according to the manufacturer's instructions. FIG. 10 shows the resulting sequence electropherogram.

Good signal intensity and the correct sequence (FIG. 6) was obtained for the experiment. A negative control in which the TO128 was omitted in the reaction gave no sequence data (data not shown) showing that a TO-dependent extension of the EO is necessary for a successful sequencing reaction.

### EXAMPLE 5

### Design of an Optimised Library of EOs and TOs for Amplification or Sequencing of Complex DNA Templates

This example shows a design and optimisation of a limited library of EOs and TOs that can effectively mimic the complexity of a 10-mer library. A complete library of 10-mers would contain 1048 576 (4¹⁰) oligonucleotides. Each oligonucleotide would be expected to have a low probability (p=0.095) of hybridising on a 100 kilobase pair DNA template. This means that a specific 10-mer is useful to target a specific site on DNA templates of this or smaller size range. Templates of this sizes are common in molecular biology and include for example, bacterial artificial chromosomes (BACs), cosmids, fosmids and many viral genomes. However, a complete library of specific 10-mers would be costly to produce and impractical.

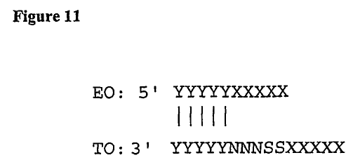

An oligonucleotide design for an EO library and a TO library is presented here as shown in FIG. 11.

A complete EO library of this design would contain 256 members and for the complete TO library 1024 oligonucleotides would be needed. The reduced size of the EO library comes from the two 3′ positions which have to be either G or C and are important for strong and efficient hybridisation and extension of the EO on the TO (Example 2). It is apparent that after TO-dependent extension of the EO a new library of extended EOs can be produced with 262 144 possible members. The members of this new library will occur with a low probability (p=0.38) on a 100 kilobase pair DNA template, thus mimicking effectively a 10-mer library. Thus with a total maximum of only 1280 oligonucleotides a partial 10-mer library of 262 144 members (or a quarter of a complete 10-mer library) can be obtained.

This design of the EO and TO can be further optimised to reduce the size of the libraries by fitting it to naturally-occurring DNA templates. Natural DNA templates (e.g. viral, procaryotic or eucaryotic genomic DNA) have a GC-content (on the molar basis) normally ranging between 30 and 70%. This would mean that members of the EO and TO library having either an unusual low (<25%) or high (>75) GC-content are unlikely to be useful and therefore can be excluded. In consequence, the library size could be halved to about 640 members without reduced coverage of most genomic sequences. In addition, oligonucleotides forming strong secondary structures with themselves (ie. intra- or inter-molecule hybridisation) could be excluded from the library.

Finally, the design of the clamp region can be considered. Sequence motifs in the clamp region that provide stringent hybridisation of the EO and TO may increase the efficiency of extension and are therefore preferable. Such motifs are known to those skilled in the art and frequently contain a high G+C content. In addition, the sequence of the clamp region should preferably contain little sequence similarity to known common motifs or sequence of the template. For example, if the target is a DNA insert within a plasmid or cosmid then a clamp design with little complementarity to the plasmid or cosmid backbone sequence will ensure that the unextended or extended EO will not hybridise to unspecific sites on the plasmid backbone. The clamp region from Example 2 (5′ ACTGG 3) is one of the possible motifs fulfilling these requirements with a free energy of binding (deltaG) of −7.8 Kcal/mol and no sequence similarity to plasmid backbones of the common pUC plasmid family.

### EXAMPLE 6

### Cycle Sequencing of DNA Using One Extendable Oligonucleotide and One Template Oligonucleotide without Klenow exo DNA polymerase, Dithiothreitol and Preincubation

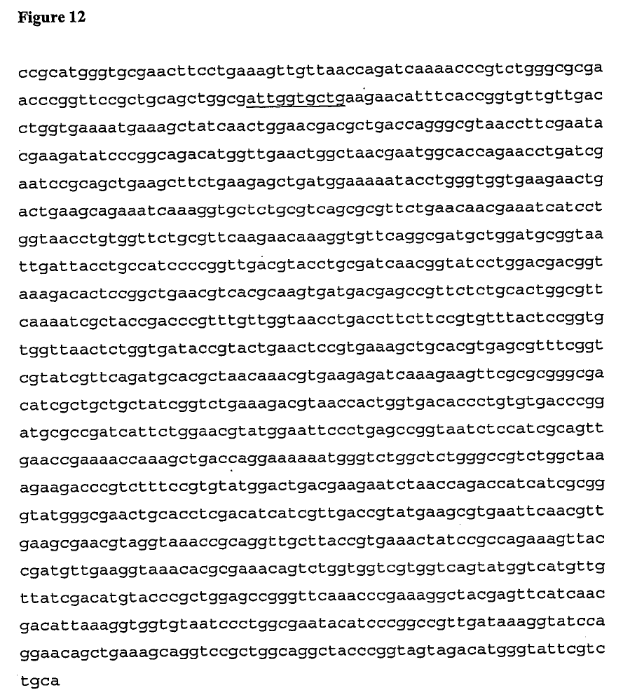

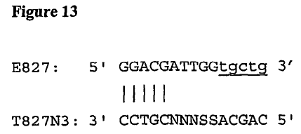

This example shows conditions for the EO/TO hybridisation and extension reaction used for a DNA cycle sequencing protocol in a single reaction without Klenow exo⁻ DNA polymerase, dithiothreitol and preincubation. This was performed using a different EO/TO pair than that used in Example 4, demonstrating the versatility of the system. The new EO/TO pair targets a genomic region of the streptomycin operon in E. coli, which was PCR amplified to give a linear sequencing template. An EO/TO primer pair was designed to hybridise to the sequence 5′-ATTGGTGCTG-3′ contained within an approximately 3300 bp region of this operon. The target region is shown underlined in FIG. 12. The EO/TO primer pair is shown in FIG. 13.

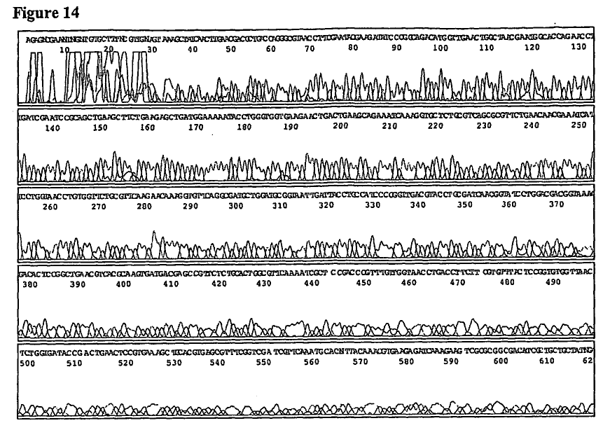

The sequencing reaction was performed using BigDye™ sequencing system version 2 (Applera Corporation, Norwalk, Conn. USA comprising Tris-HCl, magnesium chloride, AmpliTaq-FS DNA polymerase, dNTPs and fluoro-labelled ddNTPs. The reaction was supplemented with additional magnesium chloride and dGTP. The optimal sequencing reaction contained the following components 10 picomoles of EO827, 10 picomoles of TO827N3, 100 ng of the linear streptomycin operon DNA template, one microlitre of 17.5 mM MgCl sub. 2, 1 microlitres of 300 micromolar dGTP, four microlitres of the BigDye™ sequencing reagent version 2, and water to a final volume of 10 microlitres. The reaction was cycled 40 times at 96° C. for 10 sec, at 450C for 30 sec and at 60° C. for 4 min. The sequencing reaction was purified as described in Example 3. The sequencing reaction was analysed on an ABI PRISM™ 377 DNA sequencer and ABI PRISM™ sequence analysis software (Applera Corp., Norwalk, Conn., USA) according to the manufacturer's instructions. FIG. 14 shows the resulting sequence electropherogram.

Good average signal intensity and the correct sequence (FIG. 14) was obtained for the experiment. A negative control in which the T827N3 was omitted in the reaction gave no sequence data (data not shown) showing that a TO-dependent extension of the EO is necessary for a successful sequencing reaction.

Reaction Condition Optimisation

To determine the ideal magnesium chloride concentration a series of sequencing reactions was performed with the addition of one microlitre of a 0, 7.5, 12.5, 17.5, 22.5, 25, 30, 40 and 50 nM MgCl sub. 2. All other parameters were kept the same as the previous example. It was found that the optimal magnesium chloride addition is one microlitre of a 17.5 mM solution. The use of lower concentrations resulted in a reduction of sequencing signal while higher concentrations showed no further improvement.

The ABI BigDye sequencing reagent contains deoxyinosine triphosphate (dITP) in place of dGTP (BigDye version 2 and 3 manuals; Applera Corporation, Norwalk, USA) While substitution of dITP for dGTP in the BigDye mix reduces sequencing problems (such as compressions), it may present a problem for sequencing reactions described in Example 6. In the absence of dGTP in the reaction mix, dITP will be incorporated into the extended EO primer at positions opposite a cytosine residue on the TO primer with the effect of reducing the Tm of the extended EO primer. To overcome this potential problem, the addition of low concentrations of dGTP was investigated. Using a range of dGTP between 0 and 50 micromolar, supplementation with 30 micromolar dGTP was found to be optimal. Higher concentrations were found to cause more sequencing errors while lower concentrations showed reduced sequencing signal strength.

The reaction described in Example 6 was also performed using plasmid DNA where the 100 ng of linear template was replaced with 500 ng of the circular plasmid pUC4G which contains the same region of the E. coli streptomycin operon as the linear fragment. (Hou, Y, Lin, Y.-P., Sharer, D. and March, P. E., 1994). Sequencing results of similar quality were obtained from both the plasmid template and linear template.

The optimal EO to TO molar ratio was determined by varying the concentration of TO827N3 from 0.25 to 8 micromolar, while keeping the EO827 concentration constant at one micromolar. An EO to TO molar ratio of between 1:1 and 2:1 was found to give the highest quality sequencing results. Higher EO:TO ratios were found to result in less signal intensity, presumably due to inefficient extension of the EO primer in the presence of limiting amounts of the TO primer. Lower ratios (i.e. excess TO) were found to give mixed sequence signals, most likely caused by the excess TO primer binding to additional non-specific sites within the template.

The optimal concentrations of EO and TO primers in the sequencing reaction was also determined. The EO827 and TO827N3 concentration (at a 1:1 ratio) were varied between 0.25 and 8 micromolar. The optimum concentration was found to be 1 micromolar. Lower concentrations produced high quality sequence at the expense of reduced signal intensity, Higher primer concentrations produced more sequencing signal but at the expense of an increased error rate.

Finally, to determine if the sequencing reaction conditions were compatible with other sequencing chemistries, the BigDye™ sequencing reagent version 2 was replaced with the BigDye™ sequencing reagent version 3 (Applera Corporation, Norwalk, Conn. USA—the precise differences between the BigDye™ reagents in terms of their components are not supplied by the manufacturer but it is indicated that version 3 is more suitable for capillary machines such as the ABI 3700) or DYEnamic ET Terminator (Amersham Pharmacia Biotech, Piscataway N.J., USA). Good sequencing data were obtained from both chemistries, illustrating that the TO-dependent extension of the EO can occur in a variety of sequencing reagents.

### EXAMPLE 7

### Effect of the Oligonucleotides Design on the Efficiency of DNA Cycle Sequencing

This example shows which aspects of the EO and TO design are important for efficient hybridisation and extension of the EO. Various EO/TO pairs were generated and their utility tested in sequencing reactions.

The sequencing reactions were performed using BigDye™ sequencing system version 2 (Applera Corporation, Norwalk, Conn. USA). The reactions were supplemented with additional magnesium chloride and dGTP. The optimal sequencing reaction contained the following components: 10 picomoles of the EO primer, 10 picomoles of the corresponding TO primer, 100 ng of the linear streptomycin operon DNA template (Example 6), one microlitre of 17.5 mM MgCl sub. 2, 1 microlitre of 300 micromolar dGTP, four microlitres of the BigDye™ sequencing reagent version 2, and water to a final volume of 10 microlitres. The reactions were cycled 40 times at 96° C. for 10 sec, at 45° C. for 30 sec and at 60° C. for 4 min. The sequencing reactions were purified and analysed as described in Example 3.

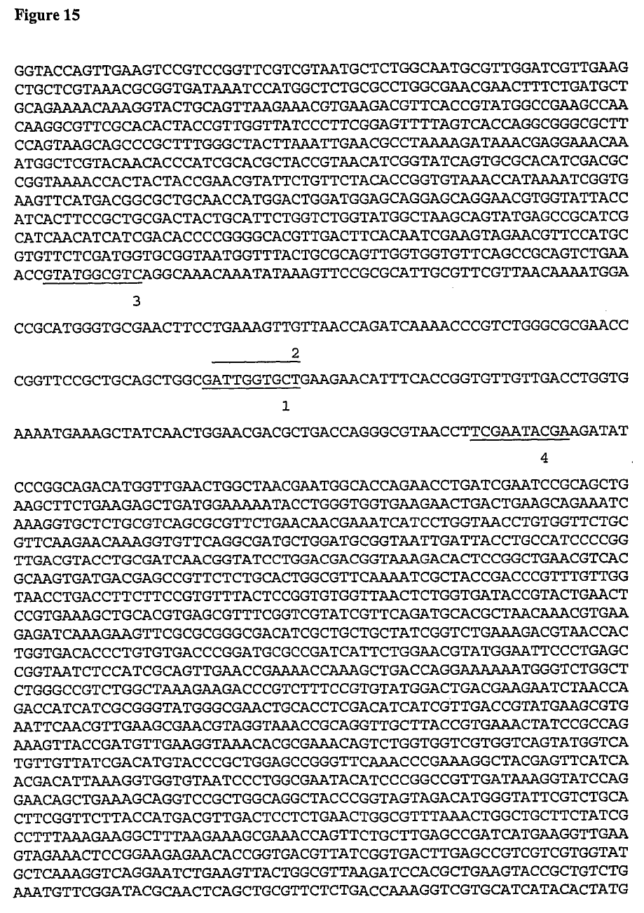

The BO and TO primers were chosen to bind to the sequences shown in FIG. 15.

Importance of Additional 3′ Adenine on the Extended EO

Non-proofreading polymerases can add an adenine residue at the 3′ end of an extension product. Most cycle sequencing applications employ non-proofreading polymerases and as a consequence the EO can have an additional 3′ adenine. A primer with an additional 3′ adenine will not be extended in the sequencing reaction unless there is a corresponding thymidine on the template sequence.

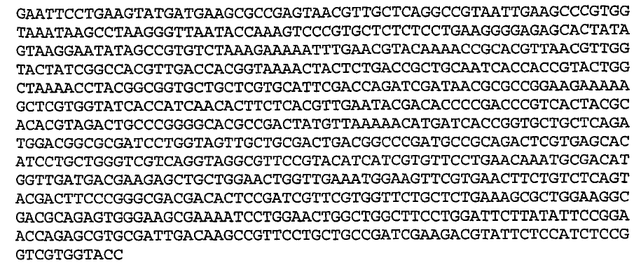

To test this hypothesis, EO and an TO primers were designed for a target site that did not contain a complementary thymine downstream of the target site (see underlined region numbered 1 in FIG. 15). The EO and TO primers are shown in FIG. 16.

A cycle sequencing reaction was performed as described previously with 10 picomoles of E826 and 10 picomoles of T626. Only very poor sequencing data was obtained, which indicates that an additional 3′A on an extended EO without a complementary position in the sequencing template prevents efficient extension during the sequencing reaction.

Hybridisation of the 3′ Prime End of the EO to the “catch” Region

Efficient hybridisation of the 3′ end of the EO to the TO is important for the successful extension of the EO. The person skilled in the art will understand that hybridisation between guanine and cytosine is more stable than between adenine and thymine. As such, EO primers containing guanine and/or cytosine on their 3′ ends should be extended more efficiently. It was predicted that the extension of the EO primer, and therefore the success of a subsequent sequencing reaction, may be dependent on the number of guanine/cytosine pairs present at the 3′end of the EO primer.

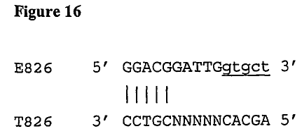

A range of EO/TO pairs were designed to test this hypothesis. These oligonucleotides pairs are shown in FIG. 17 with reference given to their specific target site shown in FIG. 15. The pair EO827/TO823N5 forms 2 guanine/cytosine pairs, the pair E686/T686N5 forms one guanine/cytosine pair and the pair E915/T915 has no such binding pair.

All three pairs were tested in a sequencing reaction under the conditions given above. The pairs EO827/TO823N5 gave good sequencing results, while for the pairs E686/T686N5 and E915/T915 sequence data of greatly reduced quality was obtained Thus, while EOs without G or C at the 3′ end can be used in the present invention, the results of this example suggest that the inclusion of two guanine/cytosine pairs at the 3′ end of the EO are important for efficient extension under the specific conditions examined. The skilled reader will be aware, however, that with careful manipulation of the reaction conditions it is likely to be possible to improve the sequencing efficiency of the primers not having two G/C pairs at the positions indicated and discussed above.

Degree of Degeneracy of the TO “catch” Region

Efficient hybridisation and extension of the EO is potentially dependent on the degree of degeneracy of the “clamp” region contained within the TO primer. EO primers designed with at least two guanine and/or cytosine 3′ position appears to be preferable and therefore the corresponding and hybridising positions in the TO primer could be restricted in their degree of degeneracy; that is an N position should be replaced by an S position (encoding either for guanine or cytosine).

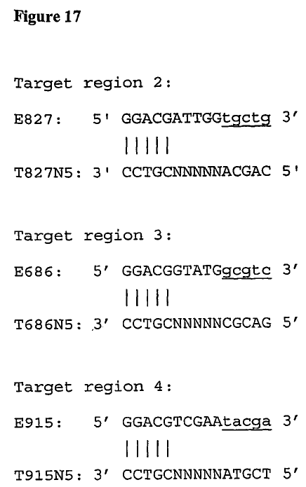

A range of TO primers with different degrees of degeneracy in the “clamp” region were designed to test this hypothesis. The TO primers examined are shown in FIG. 18 and they allow extension of E827 and binding to the target site shown in FIG. 15.

All three pairs were tested in sequencing reactions and all gave sequencing results. However, reactions in which T827N3 and T827N4 primers were used gave stronger sequencing signals than those in which the T827N5 primer was used, which indicates that a reduced degree of degeneracy (ie. S positions instead of N positions) is preferable.

### EXAMPLE 8

### Amplification of Genomic DNA Region from E. coli Using Two Extendable Oligonucleotide and Two Template Oligonucleotides with Degenerate Positions

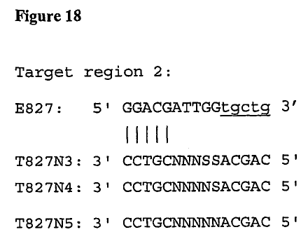

This example produces two oligonucleotides by the hybridisation and extension of two EO/TO pairs. The TO primers possess a degenerate “catch” region and are therefore suitable for other amplifications. In this example, the extended EO primers are used without further treatment in a reaction amplifying a 211 base pairs region of the Escherichia coli genome shown in FIG. 19 (NCBI accession number AE000137.1; Escherichia coli K12 MG1655 section 27 of 400 of the complete genome; position 1070-1280; intergenic region between a putative ribosomal protein and the EaeH protein (Attaching and effacing protein)).

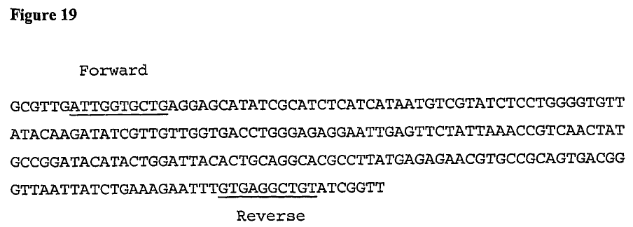

Based on the target sequence, the primers shown in FIG. 20 were designed and FIG. 20 shows how the primers hybridise as well as the extension products obtained.

The initial hybridisation and extension of the EO primers was performed in two separate reactions (for each EO/TO pair) containing the following reagents (in a final volume of 10 microlitre): 100 picomoles of EOF or EOR, 100 picomoles of TOF or TOR, 200 micromolar dNTPs (MBI Fermentas, Vilinius, Lithuania), 10 mM 50 tris(hydroxymethyl) aminomethyl hydrochloride (Tris-HCl) (pH 8.5 at 25° C.), 5 millimolar magnesium chloride (MgCl sub. 2), 1 millimolar dithiothreitol, 1 unit of Klenow exo⁻ DNA polymerase (MBI Fermentas, Vilinius, Lithuania). Negative control reactions were performed by omitting either the EO or TO primers. The reactions were incubated at 37° C. for 30 min and then for 20 min at 65° C.

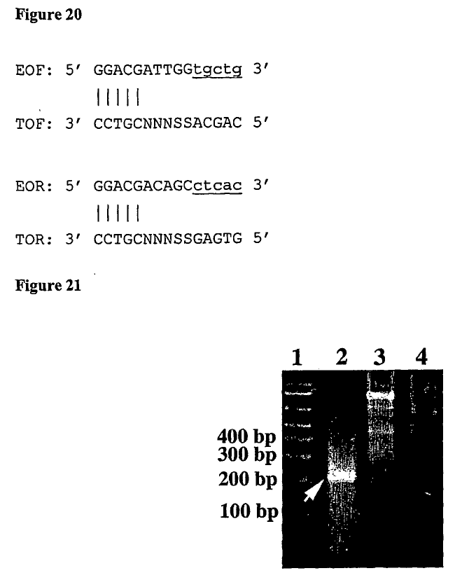

For the amplification reaction of the target DNA region the following reagents were combined: one microlitre from each of the two EO primer extension reactions, one microlitre of E. coli genomic DNA (100 ng per microlitre), three microlitres of 25 millimolar MgCl sub. 2, four microlitres of 2 millimolar dNTPs (MBI Fermentas), two microlitres of 10× buffer [100 millimolar Tris-HCl (pH 9 at 25° C.), 500 millimolar potassium chloride (KCl), 1% (vol/vol) Triton X-100 (Promega)] and water to a final volume of 16 microlitre. The mixture was heated for two minutes at 95° C. and then cooled to 80° C. at which stage four microlitres of Taq DNA polymerase (0.25 units/microlitre; Promega) were added. The reactions were then cycled 35 times at 95° C. for 10 sec and 45° C. for 30 sec. After a final heating step for 2 min at 72° C. the reactions were stored at 4° C. Five microlitres of the reactions were then separated on a 3% (w/v) agarose gel before staining with ethidium bromide using standard techniques (Sambrook et al. 2001). FIG. 21 shows the result of this experiment.

As can be seen in FIG. 21, a DNA product of approximately 210 base pairs (arrow; Lane 2) is generated when the Klenow-treated EO/TO primer pairs are used in the amplification reaction. This product is not produced when the TO primers are omitted (Lane 3; FIG. 21), demonstrating that a TO-dependent extension of the EO primers is necessary for successful amplification. Furthermore, omission of the EO primers prevented the generation of products in the expected size range (Lane 4; FIG. 21). While some non-specific products are observed when either the EO or TO primers are omitted (Lanes 3 and 4; FIG. 21), they are absent when the extended EO/TO primer pairs are used (Lane 2; FIG. 21).

From this example it would be clear to the person skilled in the art that the ability to amplify almost any specific DNA regions using two EO and TO primer pairs is possible using a limited set of primers (eg. the set described in Example 5). This makes it possible to amplify almost any DNA region from complex targets such a genomic DNA or environmental samples using this technique.

### EXAMPLE 9

### A Kit Comprising an Extendable Oligonucleotide Library (EO Library) and a Template Oligonucleotide Library (TO Library) Suitable for Sequencing DNA Fragments

The following example shows the design and synthesis of a kit comprising libraries of EO and TO primers suitable (at least) for DNA sequencing and PCR amplification.

A 256 member EO library was created using the design shown in Table 1. Some of the EO primers included an adenine replacement i.e. 2,4 diaminopurine (abbreviated by “D”) in the “catch” region. The nucleotide-analogue 2,4 diaminopurine can form three hydrogen-bonds with thymidine and provides stronger hybridisation between the complementary positions (Wu et al. 2002). Incorporation of “D” into the “catch” region both increases the affinity of the EO for the TO (potentially improving the efficiency of the EO-extension reaction) and provides greater affinity of the extended EO for the desired template sequence.

TABLE 1

A library of extendable oligonucleotides (EOs).
The left column shows a oligonucleotide
identification code and the right column shows
the sequence 5′ to 3′ from left to right.
“D” stands for 2,4 diamino purine.

| | |
|---|---|
| EO-ID | Sequence (5′ to 3′) |

| | |
|---|---|
| E001-DDDCC | CGTCCDDDCC |
| E002-DDCCC | CGTCCDDCCC |
| E003-DDGCC | CGTCCDDGCC |
| E004-DDTCC | CGTCCDDTCC |
| E005-DCDCC | CGTCCDCDCC |
| E006-DCCCC | CGTCCDCCCC |
| E007-ACGCC | CGTCCACGCC |
| E008-DCTCC | CGTCCDCTCC |
| E009-DGDCC | CGTCCDGDCC |
| E010-AGCCC | CGTCCAGCCC |
| E011-AGGCC | CGTCCAGGCC |
| E012-DGTCC | CGTCCDGTCC |
| E013-DTDCC | CGTCCDTDCC |
| E014-DTCCC | CGTCCDTCCC |
| E015-DTGCC | CGTCCDTGCC |
| E016-DTTCC | CGTCCDTTCC |
| E017-CDDCC | CGTCCCDDCC |
| E018-CDCCC | CGTCCCDCCC |
| E019-CAGCC | CGTCCCAGCC |
| E020-CDTCC | CGTCCCDTCC |
| E021-CCACC | CGTCCCCACC |
| E022-CCCCC | CGTCCCCCCC |
| E023-CCGCC | CGTCCCCGCC |
| E024-CCTCC | CGTCCCCTCC |
| E025-CGDCC | CGTCCCGDCC |
| E026-CGCCC | CGTCCCGCCC |
| E027-CGGCC | CGTCCCGGCC |
| E028-CGTCC | CGTCCCGTCC |
| E029-CDTCC | CGTCCCDTCC |
| E030-CTCCC | CGTCCCTCCC |
| E031-CTGCC | CGTCCCTGCC |
| E032-CTTCC | CGTCCCTTCC |
| E033-GDDCC | CGTCCGDDCC |
| E034-GDCCC | CGTCCGDCCC |
| E035-GDGCC | CGTCCGDGCC |
| E036-GDTCC | CGTCCGDTCC |
| E037-GCDCC | CGTCCGCDCC |
| E038-GCCCC | CGTCCGCCCC |
| E039-GCGCC | CGTCCGCGCC |
| E040-GCTCC | CGTCCGCTCC |
| E040-GCTCC | CGTCCGCTCC |
| E041-GGDCC | CGTCCGGDCC |
| E042-GGCCC | CGTCCGGCCC |
| E043-GGGCC | CGTCCGGGCC |
| E044-GGTCC | CGTCCGGTCC |
| E045-GTDCC | CGTCCGTDCC |
| E046-GTCCC | CGTCCGTCCC |
| E047-GTGCC | CGTCCGTGCC |
| E048-GTTCC | CGTCCGTTCC |
| E049-TDDCC | CGTCCTDDCC |
| E050-TDCCC | CGTCCTDCCC |
| E051-TDGCC | CGTCCTDGCC |
| E052-TDTCC | CGTCCTDTCC |
| E053-TCDCC | CGTCCTCDCC |
| E054-TCCCC | CGTCCTCCCC |
| E055-TCGCC | CGTCCTCGCC |
| E056-TCTCC | CGTCCTCTCC |
| E057-TGDCC | CGTCCTGTCC |
| E058-TGCCC | CGTCCTGCCC |
| E059-TGGCC | CGTCCTGGCC |
| E060-TGTCC | CGTCCTGTCC |
| E061-TTDCC | CGTCCTTDCC |
| E062-TTCCC | CGTCCTTCCC |
| E063-TTGCC | CGTCCTTGCC |
| E064-TTTCC | CGTCCTTTCC |
| E065-DDDCG | CGTCCDDDCG |
| E066-DDCCG | CGTCCDDCCG |
| E067-DDGCG | CGTCCDDGCG |
| E068-DDTCG | CGTCCDDTCG |
| E069-DCDCG | CGTCCDCDCG |
| E070-DCCCG | CGTCCDCCCG |
| E071-DCGCG | CGTCCDCGCG |
| E072-DCTCG | CGTCCDCTCG |
| E073-DGDCG | CGTCCDGDCG |
| E074-DGCCG | CGTCCDGCCG |
| E075-DGGCG | CGTCCDGGCG |
| E076-DGTCG | CGTCCDGTCG |
| E077-DTDCG | CGTCCDTDCG |
| E078-DTCCG | CGTCCDTCCG |
| E079-DTGCG | CGTCCDTGCG |
| E080-DTTCG | CGTCCDTTCG |
| E081-CDDCG | CGTCCCDDCG |
| E082-CDCCG | CGTCCCDCCG |
| E083-CDGCG | CGTCCCDGCG |
| E084-CDTCG | CGTCCCDTCG |
| E085-CCDCG | CGTCCCCDCG |
| E086-CCCCG | CGTCCCCCCG |
| E087-CCGCG | CGTCCCCGCG |
| E088-CCTCG | CGTCCCCTCG |
| E089-CGDCG | CGTCCCGDCG |
| E090-CGCCG | CGTCCCGCCG |
| E091-CGGCG | CGTCCCGGCG |
| E092-CGTCG | CGTCCCGTCG |
| E093-CTDCG | CGTCCCTDCG |
| E094-CTCCG | CGTCCCTCCG |
| E095-CTGCG | CGTCCCTGCG |
| E096-CTTCG | CGTCCCTTCG |
| E097-GDDCG | CGTCCGDDCG |
| E098-GDCCG | CGTCCGDCCG |
| E099-GAGCG | CGTCCGAGCG |
| E100-GDTCG | CGTCCGDTCG |
| E101-GCDCG | CGTCCGCDCG |
| E102-GCCCG | CGTCCGCCCG |
| E103-GCGCG | CGTCCGCGCG |
| E104-GCTCG | CGTCCGCTCG |
| E105-GGDCG | CGTCCGGDCG |
| E106-GGCCG | CGTCCGGCCG |
| E107-GGGCG | CGTCCGGGCG |
| E108-GGTCG | CGTCCGGTCG |
| E109-GTDCG | CGTCCGTDCG |
| E110-GTCCG | CGTCCGTCCG |
| E111-GTGCG | CGTCCGTGCG |
| E112-GTTCG | CGTCCGTTCG |
| E113-TDDCG | CGTCCTDDCG |
| E114-TDCCG | CGTCCTDCCG |
| E115-TDGCG | CGTCCTDGCG |
| E116-TDTCG | CGTCCTDTCG |
| E117-TCDCG | CGTCCTCDCG |
| E118-TCCCG | CGTCCTCCCG |
| E119-TCGCG | CGTCCTCGCG |
| E120-TCTCG | CGTCCTCTCG |
| E121-TGDCG | CGTCCTGDCG |
| E122-TGCCG | CGTCCTGCCG |
| E123-TGGCG | CGTCCTGGCG |
| E124-TGTCG | CGTCCTGTCG |
| E125-TTDCG | CGTCCTTDCG |
| E126-TTCCG | CGTCCTTCCG |
| E127-TTGCG | CGTCCTTGCG |
| E128-TTTCG | CGTCCTTTCG |
| E129-DDDGC | CGTCCDDDGC |
| E130-DDCGC | CGTCCDDCGC |
| E131-DDGGC | CGTCCDDGGC |
| E132-DDTGC | CGTCCDDTGC |
| E133-DCDGC | CGTCCDCDGC |
| E134-DCCGC | CGTCCDCCGC |
| E135-DCGGC | CGTCCDCGGC |
| E136-DCTGC | CGTCCDCTGC |
| E137-DGDGC | CGTCCDGDGC |
| E138-DGCGC | CGTCCDGCGC |
| E139-DGGGC | CGTCCDGGGC |
| E140-DGTGC | CGTCCDGTGC |
| E141-DTDGC | CGTCCDTDGC |
| E142-DTCGC | CGTCCDTCGC |
| E143-DTGGC | CGTCCDTGGC |
| E144-DTTGC | CGTCCDTTGC |
| E145-CDDGC | CGTCCCDDGC |
| E146-CDCGC | CGTCCCDCGC |
| E147-CDGGC | CGTCCCDGGC |
| E148-CDTGC | CGTCCCDTGC |
| E149-CCDGC | CGTCCCCDGC |
| E150-CCCGC | CGTCCCCCGC |
| E151-CCGGC | CGTCCCCGGC |
| E152-CCTGC | CGTCCCCTGC |
| E153-CGDGC | CGTCCCGDGC |
| E154-CGCGC | CGTCCCGCGC |
| E155-CGGGC | CGTCCCGGGC |
| E156-CGTGC | CGTCCCGTGC |
| E157-CTDGC | CGTCCCTDGC |
| E158-CTCGC | CGTCCCTCGC |
| E159-CTGGC | CGTCCCTGGC |
| E160-CTTGC | CGTCCCTTGC |
| E161-GDDGC | CGTCCGDDGC |
| E162-GDCGC | CGTCCGDCGC |
| E163-GDGGC | CGTCCGDGGC |
| E164-GDTGC | CGTCCGDTGC |
| E165-GCDGC | CGTCCGCDGC |
| E168-GCCGC | CGTCCGCCGC |
| E167-GCGGC | CGTCCGCGGC |
| E168-GCTGC | CGTCCGCTGC |
| E169-GGDGC | CGTCCGGDGC |
| E170-GGCGC | CGTCCGGCGC |
| E171-GGGGC | CGTCCGGGGC |
| E172-GGTGC | CGTCCGGTGC |
| E173-GTDGC | CGTCCGTDGC |
| E174-GTCGC | CGTCCGTCGC |
| E175-GTGGC | CGTCCGTGGC |
| E176-GTTGC | CGTCCGTTGC |
| E177-TDDGC | CGTCCTDDGC |
| E178-TDCGC | CGTCCTDCGC |
| E179-TDGGC | CGTCCTDGGC |
| E180-TDTGC | CGTCCTDTGC |
| E181-TCDGC | CGTCCTCDGC |
| E182-TCCGC | CGTCCTCCGC |
| E183-TCGGC | CGTCCTCGGC |
| E184-TCTGC | CGTCCTCTGC |
| E185-TGDGC | CGTCCTGDGC |
| E186-TGCGC | CGTCCTGCGC |
| E187-TGGGC | CGTCCTGGGC |
| E188-TGTGC | CGTCCTGTGC |
| E189-TTDGC | CGTCCTTDGC |
| E190-TTCGC | CGTCCTTCGC |
| E191-TTGGC | CGTCCTTGGC |
| E192-TTTGC | CGTCCTTTGC |
| E193-DDDGG | CGTCCDDDGG |
| E194-DDCGG | CGTCCDDCGG |
| E195-DDGGG | CGTCCDDGGG |
| E196-DDTGG | CGTCCDDTGG |
| E197-DCDGG | CGTCCDCDGG |
| E198-ACCGG | CGTCCACCGG |
| E199-ACGGG | CGTCCACGGG |
| E200-DCTGG | CGTCCDCTGG |
| E201-DGDGG | CGTCCDGDGG |
| E202-AGCGG | CGTCCAGCGG |
| E203-AGGGG | CGTCCAGGGG |
| E204-DGTGG | CGTCCDGTGG |
| E205-DTDGG | CGTCCDTDGG |
| E206-DTCGG | CGTCCDTCGG |
| E207-DTGGG | CGTCCDTGGG |
| E208-DTTGG | CGTCCDTTGG |
| E209-CDDGG | CGTCCCDDGG |
| E210-CDCGG | CGTCCCDCGG |
| E211-CAGGG | CGTCCCAGGG |
| E212-CDTGG | CGTCCCDTGG |
| E213-CCDGG | CGTCCCCDGG |
| 2214-CCCGG | CGTCCCCCGG |
| E215-CCGGG | CGTCCCCGGG |
| E216-CCTGG | CGTCCCCTGG |
| E217-CGAGG | CGTCCCGAGG |
| E218-CGCGG | CGTCCCGCGG |
| E219-CGGGG | CGTCCCGGGG |
| E220-CGTGG | CGTCCCGTGG |
| E221-CTDGG | CGTCCCTDGG |
| E222-CTCGG | CGTCCCTCGG |
| E223-CTGGG | CGTCCCTGGG |
| E224-CTTGG | CGTCCCTTGG |
| E225-GDDGG | CGTCCGDDGG |
| E226-GDCGG | CGTCCGDCGG |
| E227-GDGGG | CGTCCGDGGG |
| 2228-GDTGG | CGTCCGDTGG |
| E229-GCDGG | CGTCCGCDGG |
| E230-GCCGG | CGTCCGCCGG |
| E231-GCGGG | CGTCCGCGGG |
| E232-GCTGG | CGTCCGCTGG |
| E233-GGDGG | CGTCCGGDGG |
| E234-GGCGG | CGTCCGGCGG |
| E235-GGGGG | CGTCCGGGGG |
| E236-GGTGG | CGTCCGGTGG |
| E237-GTDGG | CGTCCGTDGG |
| E238-GTCGG | CGTCCGTCGG |
| E239-GTGGG | CGTCCGTGGG |
| E240-GTTGG | CGTCCGTTGG |
| E241-TDDGG | CGTCCTDDGG |
| E242-TDCGG | CGTCCTDCGG |
| E243-TDGGG | CGTCCTDGGG |
| E244-TDTGG | CGTCCTDTGG |
| E245-TCDGG | CGTCCTCDGG |
| E246-TCCGG | CGTCCTCCGG |
| E247-TCGGG | CGTCCTCGGG |
| E248-TCTGG | CGTCCTCTGG |
| E249-TGDGG | CGTCCTGDGG |
| E250-TGCGG | CGTCCTGCGG |
| E251-TGGGG | CGTCCTGGGG |
| E252-TGTGG | CGTCCTGTGG |
| E253-TTDGG | CGTCCTTDGG |
| E254-TTCGG | CGTCCTTCGG |
| E255-TTGGG | CGTCCTTGGG |
| E256-TTTGG | CGTCCTTTGG |

A 512 member TO library was created using the design shown in Table 2. The TO primers of this library were modified to include an additional 3′ amine-group. This 3′amine modification renders the TO non-extendable by DNA-polymerases thus preventing the extension of mishybridised TO primers and thus assisting in the prevention of incorrect sequencing data being generated.

TABLE 2

A library of extendable oligonucleotides (TOs).
The left column shows a oligonucleotide
identification code and the right column shows
the sequence (5′ to 3′).

| | |
|---|---|
| TO-ID | Sequence (5′ to 3′) |

| | |
|---|---|
| T001-GCCTG | CAGGCSSNNNGGACG |
| T002-GGCAG | CTGCCSSNNNGGACG |
| T003-GGCTG | CAGCCSSNNNGGACG |
| T004-AACGC | GCGTTSSNNNGGACG |
| T005-GCGTT | AACGCSSNNNGGACG |
| T006-ACCGT | ACGGTSSNNNGGACG |
| T007-ACGGT | ACCGTSSNNNGGACG |
| T008-CCGAC | GTCGGSSNNNGGACG |
| T009-CCGTC | GACGGSSNNNGGACG |
| T010-CGACC | GGTCGSSNNNGGACG |
| T011-CGGAC | GTCCGSSNNNGGACG |
| T012-CGGTC | GACCGSSNNNGGACG |
| T013-CGTCC | GGACGSSNNNGGACG |
| T014-GACCG | CGGTCSSNNNGGACG |
| T015-GACGG | CCGTCSSNNNGGACG |
| T016-GGACG | CGTCCSSNNNGGACG |
| T017-GGTCG | CGACCSSNNNGGACG |
| T018-GTCCG | CGGACSSNNNGGACG |
| T019-TGCCT | AGGCASSNNNGGACG |
| T020-AGGCA | TGCCTSSNNNGGACG |
| T021-ATGCG | CGCATSSNNNGGACG |
| T022-CGCAT | ATGCGSSNNNGGACG |
| T023-GAGGC | GCCTCSSNNNGGACG |
| T024-GCCTC | GAGGCSSNNNGGACG |
| T025-GCGAA | TTCGCSSNNNGGACG |
| T026-GGAGC | GCTCCSSNNNGGACG |
| T027-GGCTC | GAGCCSSNNNGGACG |
| T028-TTCGC | GCGAASSNNNGGACG |
| T029-ACCGA | TCGGTSSNNNGGACG |
| T030-ACGGA | TCCGTSSNNNGGACG |
| T031-TCCGT | ACGGASSNNNGGACG |
| T032-TCGGT | ACCGASSNNNGGACG |
| T033-AAGCG | CGCTTSSNNNGGACG |
| T034-CGCTT | AAGCGSSNNNGGACG |
| T035-CCGAG | CTCGGSSNNNGGACG |
| T036-CCTCG | CGAGGSSNNNGGACG |
| T037-CGAGG | CCTCGSSNNNGGACG |
| T038-CGGAG | CTCCGSSNNNGGACG |
| T039-CTCCG | CGGAGSSNNNGGACG |
| T040-CTCGG | CCGAGSSNNNGGACG |
| T041-GGTGG | CCACCSSNNNGGACG |
| T042-CACCC | GGGTGSSNNNGGACG |
| T043-CCACC | GGTGGSSNNNGGACG |
| T044-CCCAC | GTGGGSSNNNGGACG |
| T045-GGGTG | CACCCSSNNNGGACG |
| T046-GTGGG | CCCACSSNNNGGACG |
| T047-ATCGC | GCGATSSNNNGGACG |
| T048-GCGAT | ATCGCSSNNNGGACG |
| T049-AGGCT | AGCCTSSNNNGGACG |
| T050-GCACA | TGTGCSSNNNGGACG |
| T051-GTGCA | TGCACSSNNNGGACG |
| T052-TGCAC | GTGCASSNNNGGACG |
| T053-TGTGC | GCACASSNNNGGACG |
| T054-TCCGA | TCGGASSNNNGGACG |
| T055-TCGGA | TCCGASSNNNGGACG |
| T056-GGCAA | TTGCCSSNNNGGACG |
| T057-TTGGC | GCCAASSNNNGGACG |
| T058-ACCCA | TGGGTSSNNNGGACG |
| T059-TGGGT | ACCCASSNNNGGACG |
| T060-ACACG | CGTGTSSNNNGGACG |
| T061-ACGTG | CACGTSSNNNGGACG |
| T062-CACGT | ACGTGSSNNNGGACG |
| T063-CGTGT | ACACGSSNNNGGACG |
| T064-GACCC | GGGTCSSNNNGGACG |
| T065-GGACC | GGTCCSSNNNGGACG |
| T066-GGGAC | GTCCCSSNNNGGACG |
| T067-GGGTC | GACCCSSNNNGGACG |
| T068-GGTCC | GGACCSSNNNGGACG |
| T069-GTCCC | GGGACSSNNNGGACG |
| T070-CAGGG | CCCTGSSNNNGGACG |
| T071-CCCAG | CTGGGSSNNNGGACG |
| T072-CCCTG | CAGGGSSNNNGGACG |
| T073-CCTGG | CCAGGSSNNNGGACG |
| T074-CTGGG | CCCAGSSNNNGGACG |
| T075-AACCG | CGGTTSSNNNGGACG |
| T076-AACGG | CCGTTSSNNNGGACG |
| T077-CCGTT | AACGGSSNNNGGACG |
| T078-CGGTT | AACCGSSNNNGGACG |
| T079-GCGTA | TACGCSSNNNGGACG |
| T080-TACGC | GCGTASSNNNGGACG |
| T081-CAGCA | TGCTGSSNNNGGACG |
| T082-CTGCA | TGCAGSSNNNGGACG |
| T083-TGCAG | CTGCASSNNNGGACG |
| T084-TGCTG | CAGCASSNNNGGACG |
| T085-ACAGC | GCTGTSSNNNGGACG |
| T086-ACTGC | GCAGTSSNNNGGACG |
| T087-AGCAC | GTGCTSSNNNGGACG |
| T088-AGTGC | GCACTSSNNNGGACG |
| T089-ATGGC | GCCATSSNNNGGACG |
| T090-GCACT | AGTGCSSNNNGGACG |
| T091-GCAGT | ACTGCSSNNNGGACG |
| T092-GCCAT | ATGGCSSNNNGGACG |
| T093-GCTGT | ACAGCSSNNNGGACG |
| T094-GGCAT | ATGCCSSNNNGGACG |
| T095-GTGCT | AGCACSSNNNGGACG |
| T096-TCCCA | TGGGASSNNNGGACG |
| T097-TGGGA | TCCCASSNNNGGACG |
| T098-CACGA | TCGTGSSNNNGGACG |
| T099-CGTGA | TCACGSSNNNGGACG |
| T100-TCACG | CGTGASSNNNGGACG |
| T101-TGACG | CGTCASSNNNGGACG |
| T102-TGTCG | CGACASSNNNGGACG |
| T103-AAGGC | GCCTTSSNNNGGACG |
| T104-CGACA | TGTCGSSNNNGGACG |
| T105-CGTCA | TGACGSSNNNGGACG |
| T106-GCCTT | AAGGCSSNNNGGACG |
| T107-GGCTT | AAGCCSSNNNGGACG |
| T108-TCGTG | CACGASSNNNGGACG |
| T109-ACCCT | AGGGTSSNNNGGACG |
| T110-ACGAC | GTCGTSSNNNGGACG |
| T111-ACGTC | GACGTSSNNNGGACG |
| T112-AGGGT | ACCCTSSNNNGGACG |
| T113-GACGT | ACGTCSSNNNGGACG |
| T114-GTCGT | ACGACSSNNNGGACG |
| T115-CCCTC | GAGGGSSNNNGGACG |
| T116-CCGAA | TTCGGSSNNNGGACG |
| T117-CCTCC | GGAGGSSNNNGGACG |
| T118-CGGAA | TTCCGSSNNNGGACG |
| T119-CTCCC | GGGAGSSNNNGGACG |
| T120-TTCCG | CGGAASSNNNGGACG |
| T121-TTCGG | CCGAASSNNNGGACG |
| T122-GCAGA | TCTGCSSNNNGGACG |
| T123-GCTGA | TCAGCSSNNNGGACG |
| T124-TAGCG | CGCTASSNNNGGACG |
| T125-TGCTC | GAGCASSNNNGGACG |
| T126-CGCTA | TAGCGSSNNNGGACG |
| T127-GAGCA | TGCTCSSNNNGGACG |
| T128-GCTCA | TGAGCSSNNNGGACG |
| T129-TCAGC | GCTGASSNNNGGACG |
| T130-TCTGC | GCAGASSNNNGGACG |
| T131-TGAGC | GCTCASSNNNGGACG |
| T132-AGCAG | CTGCTSSNNNGGACG |
| T133-AGCTG | CAGCTSSNNNGGACG |
| T134-CAGCT | AGCTGSSNNNGGACG |
| T135-CTGCT | AGCAGSSNNNGGACG |
| T136-AGGGA | TCCCTSSNNNGGACG |
| T137-GACGA | TCGTCSSNNNGGACG |
| T138-GTCGA | TCGACSSNNNGGACG |
| T139-TCCCT | AGGGASSNNNGGACG |
| T140-TCGAC | GTCGASSNNNGGACG |
| T141-TCGTC | GACGASSNNNGGACG |
| T142-ACTCG | CGAGTSSNNNGGACG |
| T143-AGACG | CGTCTSSNNNGGACG |
| T144-AGTCG | CGACTSSNNNGGACG |
| T145-ATCCG | CGGATSSNNNGGACG |
| T146-ATCGG | CCGATSSNNNGGACG |
| T147-CCGAT | ATCGGSSNNNGGACG |
| T148-CGAGT | ACTCGSSNNNGGACG |
| T149-CGGAT | ATCCGSSNNNGGACG |
| T150-CTCGT | ACGAGSSNNNGGACG |
| T151-ACGAG | CTCGTSSNNNGGACG |
| T152-CGACT | AGTCGSSNNNGGACG |
| T153-CGTCT | AGACGSSNNNGGACG |
| T154-CACCA | TGGTGSSNNNGGACG |
| T155-CCACA | TGTGGSSNNNGGACG |
| T156-GCAAC | GTTGCSSNNNGGACG |
| T157-GTTGC | GCAACSSNNNGGACG |
| T158-TGGTG | CACCASSNNNGGACG |
| T159-TGTGG | CCACASSNNNGGACG |
| T160-ACACC | GGTGTSSNNNGGACG |
| T161-ACCAC | GTGGTSSNNNGGACG |
| T162-GGTGT | ACACCSSNNNGGACG |
| T163-GTGGT | ACCACSSNNNGGACG |
| T164-CCCAA | TTGGGSSNNNGGACG |
| T165-TTGGG | CCCAASSNNNGGACG |
| T166-AACCC | GGGTTSSNNNGGACG |
| T167-GGGTT | AACCCSSNNNGGACG |
| T168-CAACG | CGTTGSSNNNGGACG |
| T169-CGTTG | CAACGSSNNNGGACG |
| T170-AGAGC | GCTCTSSNNNGGACG |
| T171-AGCTC | GAGCTSSNNNGGACG |
| T172-GAGCT | AGCTCSSNNNGGACG |
| T173-GCTCT | AGAGCSSNNNGGACG |
| T174-TGCAA | TTGCASSNNNGGACG |
| T175-TTGCA | TGCAASSNNNGGACG |
| T176-CATGC | GCATGSSNNNGGACG |
| T177-GCATG | CATGCSSNNNGGACG |
| T178-CGAGA | TCTCGSSNNNGGACG |
| T179-CTCGA | TCGAGSSNNNGGACG |
| T180-TCGAG | CTCGASSNNNGGACG |
| T181-TCTCG | CGAGASSNNNGGACG |
| T182-CCGTA | TACGGSSNNNGGACG |
| T183-CGGTA | TACCGSSNNNGGACG |
| T184-GACCA | TGGTCSSNNNGGACG |
| T185-GGACA | TGTCCSSNNNGGACG |
| T186-GGTCA | TGACCSSNNNGGACG |
| T187-GGTGA | TCACCSSNNNGGACG |
| T188-GTCCA | TGGACSSNNNGGACG |
| T189-GTGGA | TCCACSSNNNGGACG |
| T190-TACCG | CGGTASSNNNGGACG |
| T191-TACGG | CCGTASSNNNGGACG |
| T192-TCACC | GGTGASSNNNGGACG |
| T193-TCCAC | GTGGASSNNNGGACG |
| T194-TGACC | GGTCASSNNNGGACG |
| T195-TGGAC | GTCCASSNNNGGACG |
| T196-TGGTC | GACCASSNNNGGACG |
| T197-TGTCC | GGACASSNNNGGACG |
| T198-CAAGC | GCTTGSSNNNGGACG |
| T199-CTTGC | GCAAGSSNNNGGACG |
| T200-GCAAG | CTTGCSSNNNGGACG |
| T201-GCTTG | CAAGCSSNNNGGACG |
| T202-ACAGG | CCTGTSSNNNGGACG |
| T203-ACCAG | CTGGTSSNNNGGACG |
| T204-ACCTG | CAGGTSSNNNGGACG |
| T205-ACTGG | CCAGTSSNNNGGACG |
| T206-AGGTG | CACCTSSNNNGGACG |
| T207-AGTGG | CCACTSSNNNGGACG |
| T208-ATGGG | CCCATSSNNNGGACG |
| T209-CACCT | AGGTGSSNNNGGACG |
| T210-CAGGT | ACCTGSSNNNGGACG |
| T211-CTGGT | ACCAGSSNNNGGACG |
| T212-AACGT | ACGTTSSNNNGGACG |
| T213-ACGTT | AACGTSSNNNGGACG |
| T214-GGGAA | TTCCCSSNNNGGACG |
| T215-TTCCC | GGGAASSNNNGGACG |
| T216-TGCAT | ATGCASSNNNGGACG |
| T217-ATGCA | TGCATSSNNNGGACG |
| T218-AAGGG | CCCTTSSNNNGGACG |
| T219-CCCTT | AAGGGSSNNNGGACG |
| T220-CGAAC | GTTCGSSNNNGGACG |
| T221-CGTTC | GAACGSSNNNGGACG |
| T222-GAACG | CGTTCSSNNNGGACG |
| T223-GTTCG | CGAACSSNNNGGACG |
| T224-GCCTA | TAGGCSSNNNGGACG |
| T225-GGCTA | TAGCCSSNNNGGACG |
| T226-AAGCA | TGCTTSSNNNGGACG |
| T227-AGCAA | TTGCTSSNNNGGACG |
| T228-GATGC | GCATCSSNNNGGACG |
| T229-GCATC | GATGCSSNNNGGACG |
| T230-TGCTT | AAGCASSNNNGGACG |
| T231-TTGCT | AGCAASSNNNGGACG |
| T232-CAGGA | TCCTGSSNNNGGACG |
| T233-CCAGA | TCTGGSSNNNGGACG |
| T234-CCTCA | TGAGGSSNNNGGACG |
| T235-CCTGA | TCAGGSSNNNGGACG |
| T236-CTCCA | TGGAGSSNNNGGACG |
| T237-CTGGA | TCCAGSSNNNGGACG |
| T238-GAAGC | GCTTCSSNNNGGACG |
| T239-GCTTC | GAAGCSSNNNGGACG |
| T240-TCAGG | CCTGASSNNNGGACG |
| T241-TCCAG | CTGGASSNNNGGACG |
| T242-TCCTG | CAGGASSNNNGGACG |
| T243-TCTGG | CCAGASSNNNGGACG |
| T244-TGAGG | CCTCASSNNNGGACG |
| T245-TGGAG | CTCCASSNNNGGACG |
| T245-AACGA | TCGTTSSNNNGGACG |
| T247-ACCTC | GAGGTSSNNNGGACG |
| T248-ACGAA | TTCGTSSNNNGGACG |
| T249-ACTCC | GGAGTSSNNNGGACG |
| T250-AGACC | GGTCTSSNNNGGACG |
| T251-AGGAC | GTCCTSSNNNGGACG |
| T252-AGTCC | GGACTSSNNNGGACG |
| T253-ATCCC | GGGATSSNNNGGACG |
| T254-GACCT | AGGTCSSNNNGGACG |
| T255-GAGGT | ACCTCSSNNNGGACG |
| T256-GGACT | AGTCCSSNNNGGACG |
| T257-GGAGT | ACTCCSSNNNGGACG |
| T258-GGGAT | ATCCCSSNNNGGACG |
| T259-GGTCT | AGACCSSNNNGGACG |
| T260-GTCCT | AGGACSSNNNGGACG |
| T261-TCGTT | AACGASSNNNGGACG |
| T262-TTCGT | ACGAASSNNNGGACG |
| T263-AGGTC | GACCTSSNNNGGACG |
| T264-CATCG | CGATGSSNNNGGACG |
| T265-CGATG | CATCGSSNNNGGACG |
| T266-CACAC | GTGTGSSNNNGGACG |
| T267-GTGTG | CACACSSNNNGGACG |
| T268-AGCAT | ATGCTSSNNNGGACG |
| T269-CGAAG | CTTCGSSNNNGGACG |
| T270-CTTCG | CGAAGSSNNNGGACG |
| T271-ATGCT | AGCATSSNNNGGACG |
| T272-CAACC | GGTTGSSNNNGGACG |
| T273-CCAAC | GTTGGSSNNNGGACG |
| T274-AAGCT | AGCTTSSNNNGGACG |
| T275-ACGAT | ATCGTSSNNNGGACG |
| T276-ATCGT | ACGATSSNNNGGACG |
| T277-ACACA | TGTGTSSNNNGGACG |
| T278-TGTGT | ACACASSNNNGGACG |
| T279-TCCTC | GAGGASSNNNGGACG |
| T280-TCGAA | TTCGASSNNNGGACG |
| T281-AGAGG | CCTCTSSNNNGGACG |
| T282-AGGAG | CTCCTSSNNNGGACG |
| T283-CCTCT | AGAGGSSNNNGGACG |
| T284-CGATC | GATCGSSNNNGGACG |
| T285-CTCCT | AGGAGSSNNNGGACG |
| T286-GATCG | CGATCSSNNNGGACG |
| T287-GGGTA | TACCCSSNNNGGACG |
| T288-TACCC | GGGTASSNNNGGACG |
| T289-GCAAA | TTTGCSSNNNGGACG |
| T290-AACCA | TGGTTSSNNNGGACG |
| T291-ACCAA | TTGGTSSNNNGGACG |
| T292-CGTAC | GTACGSSNNNGGACG |
| T293-GACAC | GTGTCSSNNNGGACG |
| T294-GTACG | CGTACSSNNNGGACG |
| T295-GTCAC | GTGACSSNNNGGACG |
| T296-GTGAC | GTCACSSNNNGGACG |
| T297-GTGTC | GACACSSNNNGGACG |
| T298-CACAG | CTGTGSSNNNGGACG |
| T299-CACTG | CAGTGSSNNNGGACG |
| T300-CAGTG | CACTGSSNNNGGACG |
| T301-CATGG | CCATGSSNNNGGACG |
| T302-CCATG | CATGGSSNNNGGACG |
| T303-CTGTG | CACAGSSNNNGGACG |
| T304-GAACC | GGTTCSSNNNGGACG |
| T305-GGAAC | GTTCCSSNNNGGACG |
| T306-GGTTC | GAACCSSNNNGGACG |
| T307-GTTCC | GGAACSSNNNGGACG |
| T308-CAAGG | CCTTGSSNNNGGACG |
| T309-CCAAG | CTTGGSSNNNGGACG |
| T310-CCTTG | CAAGGSSNNNGGACG |
| T311-CTTGG | CCAAGSSNNNGGACG |
| T312-AAACG | CGTTTSSNNNGGACG |
| T313-TCGAT | ATCGASSNNNGGACG |
| T314-ATCGA | TCGATSSNNNGGACG |
| T315-GCTAC | GTAGCSSNNNGGACG |
| T316-GTAGC | GCTACSSNNNGGACG |
| T317-TCACA | TGTGASSNNNGGACG |
| T318-TGACA | TGTCASSNNNGGACG |
| T319-TGTCA | TGACASSNNNGGACG |
| T320-TGTGA | TCACASSNNNGGACG |
| T321-ACGTA | TACGTSSNNNGGACG |
| T322-TACGT | ACGTASSNNNGGACG |
| T323-GCAAT | ATTGCSSNNNGGACG |
| T324-GCATT | AATGCSSNNNGGACG |
| T325-AATGC | GCATTSSNNNGGACG |
| T326-ATTGC | GCAATSSNNNGGACG |
| T327-ACAGT | ACTGTSSNNNGGACG |
| T328-ACCAT | ATGGTSSNNNGGACG |
| T329-ACTGT | ACAGTSSNNNGGACG |
| T330-AGTGT | ACACTSSNNNGGACG |
| T331-ATGGT | ACCATSSNNNGGACG |
| T332-ACACT | AGTGTSSNNNGGACG |
| T333-TCCAA | TTGGASSNNNGGACG |
| T334-TGGAA | TTCCASSNNNGGACG |
| T335-TTCCA | TGGAASSNNNGGACG |
| T336-TTGGA | TCCAASSNNNGGACG |
| T337-AAAGC | GCTTTSSNNNGGACG |
| T338-CACTC | GAGTGSSNNNGGACG |
| T339-CAGAC | GTCTGSSNNNGGACG |
| T340-CAGTC | GACTGSSNNNGGACG |
| T341-CATCC | GGATGSSNNNGGACG |
| T342-CCATC | GATGGSSNNNGGACG |
| T343-CGTAG | CTACGSSNNNGGACG |
| T344-CTACG | CGTAGSSNNNGGACG |
| T345-CTCAC | GTGAGSSNNNGGACG |
| T346-CTGAC | GTCAGSSNNNGGACG |
| T347-CTGTC | GACAGSSNNNGGACG |
| T348-GACAG | CTGTCSSNNNGGACG |
| T349-GACTG | CAGTCSSNNNGGACG |
| T350-GAGTG | CACTCSSNNNGGACG |
| T351-GATGG | CCATCSSNNNGGACG |
| T352-GGATG | CATCCSSNNNGGACG |
| T353-GTCAG | CTGACSSNNNGGACG |
| T354-GTCTG | CAGACSSNNNGGACG |
| T355-GTGAG | CTCACSSNNNGGACG |
| T356-AACCT | AGGTTSSNNNGGACG |
| T357-AAGGT | ACCTTSSNNNGGACG |
| T358-ACCTT | AAGGTSSNNNGGACG |
| T359-AGGTT | AACCTSSNNNGGACG |
| T360-TAGCA | TGCTASSNNNGGACG |
| T361-TGCTA | TAGCASSNNNGGACG |
| T362-CCTTC | GAAGGSSNNNGGACG |
| T363-CGAAA | TTTCGSSNNNGGACG |
| T364-CTTCC | GGAAGSSNNNGGACG |
| T365-GAAGG | CCTTCSSNNNGGACG |
| T366-GGAAG | CTTCCSSNNNGGACG |
| T367-TACGA | TCGTASSNNNGGACG |
| T368-TCGTA | TACGASSNNNGGACG |
| T369-CTAGC | GCTAGSSNNNGGACG |
| T370-GCTAG | CTAGCSSNNNGGACG |
| T371-ACAGA | TCTGTSSNNNGGACG |
| T372-ACTCA | TGAGTSSNNNGGACG |
| T373-ACTGA | TCAGTSSNNNGGACG |
| T374-AGACA | TGTCTSSNNNGGACG |
| T375-AGTCA | TGACTSSNNNGGACG |
| T376-AGTGA | TCACTSSNNNGGACG |
| T377-ATCCA | TGGATSSNNNGGACG |
| T378-ATGGA | TCCATSSNNNGGACG |
| T379-TCCAT | ATGGASSNNNGGACG |
| T380-TGACT | AGTCASSNNNGGACG |
| T381-TGTCT | AGACASSNNNGGACG |
| T382-GACTC | GAGTCSSNNNGGACG |
| T383-GAGTC | GACTCSSNNNGGACG |
| T384-GATCC | GGATCSSNNNGGACG |
| T385-GGATC | GATCCSSNNNGGACG |
| T386-GTCTC | GAGACSSNNNGGACG |
| T387-CTCTG | CAGAGSSNNNGGACG |
| T388-CAGAG | CTCTGSSNNNGGACG |
| T389-CTCAG | CTGAGSSNNNGGACG |
| T390-CTGAG | CTCAGSSNNNGGACG |
| T391-AATCG | CGATTSSNNNGGACG |
| T392-ATTCG | CGAATSSNNNGGACG |
| T393-CGAAT | ATTCGSSNNNGGACG |
| T394-CGATT | AATCGSSNNNGGACG |
| T395-GGTAC | GTACCSSNNNGGACG |
| T396-GTACC | GGTACSSNNNGGACG |
| T397-CAACA | TGTTGSSNNNGGACG |
| T398-CACAA | TTGTGSSNNNGGACG |
| T399-TGTTG | CAACASSNNNGGACG |
| T400-TTGTG | CACAASSNNNGGACG |
| T401-AACAC | GTGTTSSNNNGGACG |
| T402-ACAAC | GTTGTSSNNNGGACG |
| T403-AGCTA | TAGCTSSNNNGGACG |
| T404-GTGTT | AACACSSNNNGGACG |
| T405-GTTGT | ACAACSSNNNGGACG |
| T406-TAGCT | AGCTASSNNNGGACG |
| T407-CCAAA | TTTGGSSNNNGGACG |
| T408-TCAGA | TCTGASSNNNGGACG |
| T409-TCTCA | TGAGASSNNNGGACG |
| T410-TCTGA | TCAGASSNNNGGACG |
| T411-TGAGA | TCTCASSNNNGGACG |
| T412-TACCA | TGGTASSNNNGGACG |
| T413-TGGTA | TACCASSNNNGGACG |
| T414-ACTCT | AGAGTSSNNNGGACG |
| T415-AGACT | AGTCTSSNNNGGACG |
| T416-AGAGT | ACTCTSSNNNGGACG |
| T417-AGGAT | ATCCTSSNNNGGACG |
| T418-AGTCT | AGACTSSNNNGGACG |
| T419-ATCCT | AGGATSSNNNGGACG |
| T420-ACATG | CATGTSSNNNGGACG |
| T421-ATGTG | CACATSSNNNGGACG |
| T422-CACAT | ATGTGSSNNNGGACG |
| T423-CATGT | ACATGSSNNNGGACG |
| T424-CTCTC | GAGAGSSNNNGGACG |
| T425-GAGAG | CTCTCSSNNNGGACG |
| T426-CCTAC | GTAGGSSNNNGGACG |
| T427-CGTAA | TTACGSSNNNGGACG |
| T428-CGTTA | TAACGSSNNNGGACG |
| T429-CTACC | GGTAGSSNNNGGACG |
| T430-GAACA | TGTTCSSNNNGGACG |
| T431-GACAA | TTGTCSSNNNGGACG |
| T432-GGTAG | CTACCSSNNNGGACG |
| T433-GTAGG | CCTACSSNNNGGACG |
| T434-GTCAA | TTGACSSNNNGGACG |
| T435-GTGAA | TTCACSSNNNGGACG |
| T436-GTTCA | TGAACSSNNNGGACG |
| T437-GTTGA | TCAACSSNNNGGACG |
| T438-TAACG | CGTTASSNNNGGACG |
| T439-TCAAC | GTTGASSNNNGGACG |
| T440-TGAAC | GTTCASSNNNGGACG |
| T441-TGTTC | GAACASSNNNGGACG |
| T442-TTACG | CGTAASSNNNGGACG |
| T443-TTCAC | GTGAASSNNNGGACG |
| T444-TTGAC | GTCAASSNNNGGACG |
| T445-TTGTC | GACAASSNNNGGACG |
| T446-AACAG | CTGTTSSNNNGGACG |
| T447-AACTG | CAGTTSSNNNGGACG |
| T448-AAGTG | CACTTSSNNNGGACG |
| T449-AATGG | CCATTSSNNNGGACG |
| T450-ACAAG | CTTGTSSNNNGGACG |
| T451-ACTTG | CAAGTSSNNNGGACG |
| T452-AGTTG | CAACTSSNNNGGACG |
| T453-ATTGG | CCAATSSNNNGGACG |
| T454-CAACT | AGTTGSSNNNGGACG |
| T455-CAAGT | ACTTGSSNNNGGACG |
| T456-CCAAT | ATTGGSSNNNGGACG |
| T457-CCATT | AATGGSSNNNGGACG |
| T458-CTGTT | AACAGSSNNNGGACG |
| T459-GCATA | TATGCSSNNNGGACG |
| T460-TATGC | GCATASSNNNGGACG |
| T461-GGAAA | TTTCCSSNNNGGACG |
| T462-AGAGA | TCTCTSSNNNGGACG |
| T463-TCTCT | AGAGASSNNNGGACG |
| T464-GCTAA | TTAGCSSNNNGGACG |
| T465-GCTTA | TAAGCSSNNNGGACG |
| T466-TAAGC | GCTTASSNNNGGACG |
| T467-TTAGC | GCTAASSNNNGGACG |
| T468-ACCTA | TAGGTSSNNNGGACG |
| T469-AGGTA | TACCTSSNNNGGACG |
| T470-TACCT | AGGTASSNNNGGACG |
| T471-TAGGT | ACCTASSNNNGGACG |
| T472-CATCA | TGATGSSNNNGGACG |
| T473-CATGA | TCATGSSNNNGGACG |
| T474-TGATG | CATCASSNNNGGACG |
| T475-ATACG | CGTATSSNNNGGACG |
| T476-ACATC | GATGTSSNNNGGACG |
| T477-ATCAC | GTGATSSNNNGGACG |
| T478-ATGAC | GTCATSSNNNGGACG |
| T479-ATGTC | GACATSSNNNGGACG |
| T480-CAAGA | TCTTGSSNNNGGACG |
| T481-CAGAA | TTCTGSSNNNGGACG |
| T482-CCTAG | CTAGGSSNNNGGACG |
| T483-CTAGG | CCTAGSSNNNGGACG |
| T484-CTCAA | TTGAGSSNNNGGACG |
| T485-CTGAA | TTCAGSSNNNGGACG |
| T486-CTTCA | TGAAGSSNNNGGACG |
| T487-CTTGA | TCAAGSSNNNGGACG |
| T488-TCAAG | CTTGASSNNNGGACG |
| T489-TCTTG | CAAGASSNNNGGACG |
| T490-TGAAG | CTTCASSNNNGGACG |
| T491-TTCAG | CTGAASSNNNGGACG |
| T492-TTCTG | CAGAASSNNNGGACG |
| T493-TTGAG | CTCAASSNNNGGACG |
| T494-AACTC | GAGTTSSNNNGGACG |
| T495-AAGAC | GTCTTSSNNNGGACG |
| T496-AAGTC | GACTTSSNNNGGACG |
| T497-ACTTC | GAAGTSSNNNGGACG |
| T498-AGAAC | GTTCTSSNNNGGACG |
| T499-AGTTC | GAACTSSNNNGGACG |
| T500-ATTCC | GGAATSSNNNGGACG |
| T501-ATAGC | GCTATSSNNNGGACG |
| T502-TAGGA | TCCTASSNNNGGACG |
| T503-TCCTA | TAGGASSNNNGGACG |
| T504-CGATA | TATCGSSNNNGGACG |
| T505-GATCA | TGATCSSNNNGGACG |
| T506-GATGA | TCATCSSNNNGGACG |
| T507-AGATG | CATCTSSNNNGGACG |
| T508-ATCAG | CTGATSSNNNGGACG |
| T509-ATCTG | CAGATSSNNNGGACG |
| T510-ATGAG | CTCATSSNNNGGACG |
| T511-GTACA | TGTACSSNNNGGACG |
| T512-GTGTA | TACACSSNNNGGACG |

By careful selection of EO/TO primer pairs from these two libraries a total of 131,072 (256×512) different extended EOs can be generated. This number represents 3.23 % of all possible 4,194,304 11-mers (4 to the power of 11=4194304) (Note: the extended EO has a specificity of 11 positions because of the additional adenine on the 3′ end). In a DNA sequence a specific 11-mer should be represented by an extended EO generated from the above-mentioned library on average every 31 nucleotide positions. Computer simulations performed on large DNA sequence data sets selected from the GenBank database suggests that this is sufficient to enable the complete sequencing by primer walking of nearly all DNA fragments.

Reactions conditions, such as reagents and temperature cycling, were optimised for use with the previously mentioned library to provide maximal success. The best reaction conditions were found to be: 10 pmol of EO primer, 10 pmol of TO primer, 4 microlitres of Big Dye™ version 2 or 3, 1 microlitre of 300 micromolar dGTP, 1 microlitre of 17.5 millimolar magnesium chloride, DNA template (appr. 100 ng for each 3 Kb of linear template and appr. 200 ng for each 3 Kb of circular template), and water to a final volume of 10 microlitres. The cycling conditions that provided the best results were: 96° C. for 2 min followed by 40 cycles of 96° C. for 10 s, 41° C. for 30 s and 60° C. for 4 min. The sequencing products were cleaned and analysed using standard protocols known to those skilled in the field (eg. those provided in Sambrook et al. 2001).

### EXAMPLE 10

### Use of the Oligonucleotide Library from Example 9 to Sequence DNA

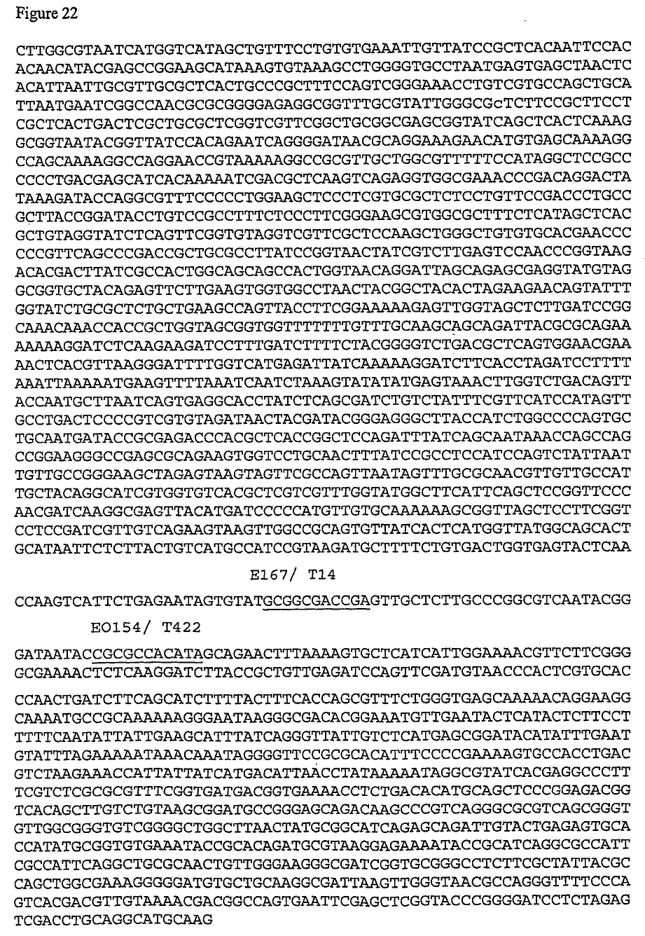

This example shows the use of an oligonucleotide library as described in Example 9 in a DNA sequencing application. Two different EO/TO pairs were chosen from the libraries of Tables 1 and 2: E154/T422 and E167/T14. These pairs were used to sequence linear pUC19 DNA. FIG. 22 shows the sequence of pUC19 DNA with the binding-sites of the extended EOs.

The sequencing reaction contained the following components: 10 picomoles of the EO primer, 10 picomoles of TO primer, 250 ng of the linear pUC19 DNA template, one microlitre of 17.5 mM MgCl sub. 2, 1 microlitre of 300 micromolar dGTP, four microlitres of the BigDye™ sequencing reagent version 2, and water to a final volume of 10 microlitres. The reactions were cycled 40 times at 96° C. for 10 sec, at 41° C. for 30 sec and at 60° C. for 4 min. The sequencing reactions were purified and analysed as described in Example 3.

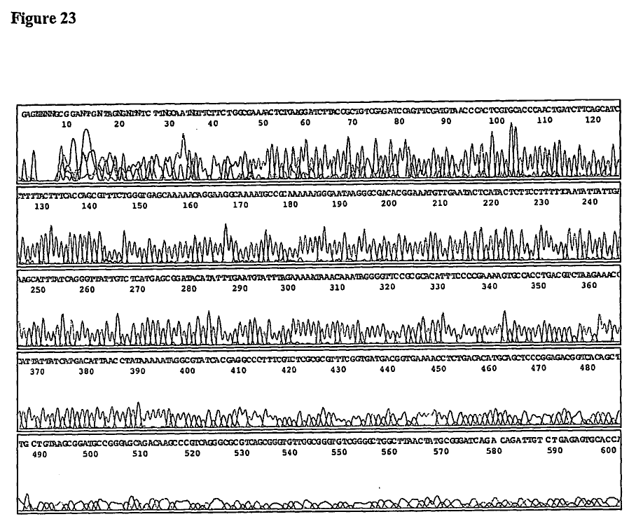

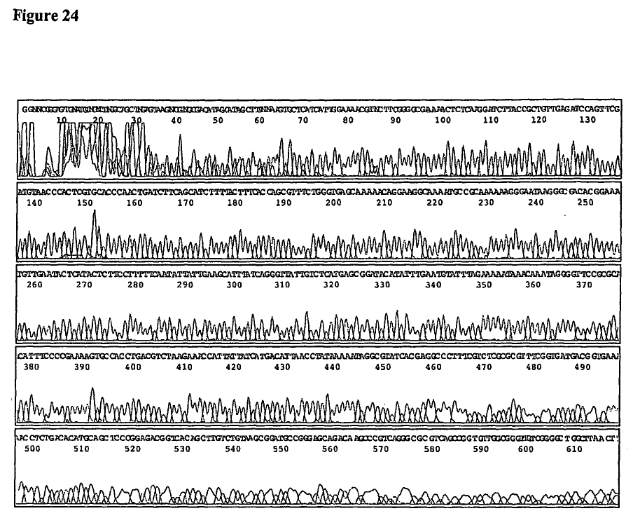

All sequencing reactions were successful and FIGS. 23 and 24 show the resulting sequence electropherograms for the pairs E154/T422 and E167/T14, respectively.

Although the invention has been described with reference to specific examples, it will be appreciated by those skilled in the art that the invention may be embodied in many other forms.

### REFERENCES

Brownstein, M J, Carpten, J D and Smith J R 1996: Modulation of non-template nucleotide addition by Taq DNA polymerase: primer modification that facilitate genotyping. BioTechniques 20, 1004-1010
Guatelli J C, Whitfield K M, Kwoh D Y, Barringer K J, Richman D D, Gingeras T R, 1990: Isothermal, in vitro amplification of nucleic acids by a multienzyme reaction modeled after retroviral replication. Proc Natl Acad Sci U S A 87, 1874-8.
Hou, Y, Lin, Y.-P., Sharer, D. and March, P. E. (1994) In vivo selection of conditional-lethal mutations in the gene encoding elongation factor G of Escherichia coli. Journal of Bacteriology 176: 123-129
Jones, L. B., Hardin, S. H. 1998 Octamer-primed cycle sequencing using dye-terminator chemistry. Nucleic Acid Research 26, 2824-2826
Kieleczawa, J., J. J. Dunn and F. W. Studier. 1992. “DNA sequencing by primer walking with strings of contiguous hexamers” Science 258, 1787-1791;
Little M C, Andrews J, Moore R, Bustos S, Jones L, Embres C, Durmowicz G, Harris J, Berger D, Yanson K, Rostkowski C, Yursis D, Price 3, Fort T, Walters A, Collis M, Llorin O, Wood J, Failing F, O'Keefe C, Scrivens B, Pope B, Hansen T, Marino K, Williams K, Boenisch, M, 1999: Strand displacement amplification and homogeneous real-time detection incorporated in a second-generation DNA probe system, BDProbeTecET. Clin Chem 45, 777-84.
Lizardi P M, Huang X, Zhu Z, Bray-Ward P, Thomas D C, Ward D C, 1998: Mutation detection and single-molecule counting using isothermal rolling-circle amplification. Nat Genet 19, 225-32.
Magnuson V L, Ally D S, Nylund S J, Karanjawalam Z E, Rayman J B, Knapp J I, Lowe A L, Gosh S and Collins F S, 1996: Substrate nucleotide-determined non-template addition of adenine by Taq DNA polymerase: implications for PCR-based genotyping and cloning. BioTechniques 21, 700-709.
Sambrook J, Fritsch E F, Maniatis T: Molecular cloning. A laboratory manual. Cold Spring Harbour Press, 1989.
Sambrook, J., P. MacCallum, D. Russell. 2001. Molecular Cloning: a laboratory manual. Cold Spring Harbor Laboratory Press, NY.
Sanger, F., Nicklen, S. and Coulson, A. R. 1977. DNA sequencing with chain-terminating inhibitors. Proc. Natl. Acad. Sci. 74, 5464-5467
Santa Lucia, J. 1988. A unified view of polymer, dumbbell, and oligonucleotide DNA nearest-neighbor thermodynamics. Proc Natl Acad Sci USA 95: 1460-1465.
Slightom, J. L., J. H. Bock, D. R. Siemieniak, G. D. Hurst, K. L. Beattie. 1994. Nucleotide sequencing double-stranded plasmids with primers selected from a nonamer library. BioTechniques 17, 536-544
Strauss, E. C., J. A. Kabori, G. Sui, and L. E. Hood. 1986 Specific-primer-directed DNA sequencing. Anal. Biochem. 154, 353-360
Tillett D, Neilan B A. 1999. n-butanol purification of dye terminator sequencing reactions. Biotechniques 26(4), 606-608
Wiedmann M, Wilson W J, Czajka J, Luo J, Barany F, Batt C A, 1994:Ligase chain reaction (LCR)—overview and applications. PCR Methods Appl 3, S51-64.
Wu X, Delgado G, Krishnamurthy R, Eschenmoser A, 2002: 2,6-Diaminopurine in TNA: Effect on Duplex Stabilities and on the Efficiency of Template-Controlled Ligations. Org Lett. 4(8), 1283-1286.

The claims defining the invention are as follows:

1. A method of increasing the affinity of an extendable oligonucleotide (EO) for a target nucleic acid comprising:
(a) hybridisation of the EO to a template oligonucleotide (TO) via a region of complementarity, wherein the 5′ region of the TO
(i) overhangs the 3′ end of the EO; and
(ii) bears homology to the target nucleic acid; and
(b) extension of the EO such that at least one nucleotide complementary to the TO is added to the 3′ end of the EO, resulting in an extended EO.

2. A method according to claim 1 wherein the EO is of equal or shorter length than the TO.

3. A method according to claim 1 or claim 2 wherein the EO and TO comprise deoxyribonucleic acids.

4. A method according to claim 1 wherein the 5′ region of the TO overhangs the 3′end of the EO by two to six nucleic acids.

5. A method according to claim 1 wherein extension of the EO is achieved by a polymerase.

6. A method according to claim 5 wherein the polymerase is selected from the following: E. coli DNA polymerase I, the Klenow fragment of E. coli DNA polymerase, Vent DNA polymerase, Vent (exo⁻), Deep Vent, Deep vent (exo⁻), 9.degree. N DNA polymerase, T4 DNA polymerase, T7 DNA polymerase, T7 RNA polymerase, M-MuLV reverse transcriptase, SP6 RNA polymerase or Taq DNA polymerase.

7. A method according to claim 5 or claim 6 wherein the polymerase has no 5′ to 3′ or 3′ to 5′ exonuclease activities.

8. A method according to claim 7 wherein the polymerase is Klenow 3′ to 5′ exonuclease minus.

9. A method according to claim 1 wherein the extended EO is purified.

10. A method according to claim 1 wherein the extended EO is dissociated from the TO and used to bind to the target nucleic acid in a further method.

11. A method according to claim 10 wherein the further method is selected from the following: polymerase chain reaction (PCR), ligation chain reaction (LCR), reverse-transcriptase PCR (T-PCR), primer extension reaction for mRNA-transcript analysis, self-sustaining sequence replication, rolling circle amplification, strand displacement amplification, isothermal DNA amplification and DNA-sequencing.

12. A method according to claim 1 wherein the 3′ end of the TO is extendable by a polymerase.

13. A method according to claim 1 wherein extension of the TO is blocked.

14. A method according to claim 13 wherein extension of the TO is blocked by a TO design that creates a non-hybridising 5′ overhang of the EO, providing no template for the extension of the TO.

15. A method according to claim 13 wherein extension of the TO is blocked by modification of the 3′ end of the TO rendering it unrecognisable or non-extendable by a polymerase.

16. A method according to claim 15 wherein the modification of the 3′ end of the TO is by addition of a phosphate group, biotin, carbon chain, amine or dideoxyribonucleotide to the 3′ end of the TO.

17. A method according to claim 1 wherein the EO and/or TO comprise a degenerate or universal nucleotide.

18. A method according to claim 17 wherein the universal nucleotide is selected from the following: inosine, 3-nitropyrrole and 5-nitroindole.

19. A method of amplifying a target nucleic acid comprising
(a) hybridisation of an extendable oligonucleotide (BO), to a template oligonucleotide (TO), wherein the 5′ region of the TO
(i) overhangs the EO by at least one nucleotide; and
(ii) bears homology to the target nucleic acid;
(b) extension of the EO such that at least one nucleotide complementary to the TO is added to the 3′ end of the EO; and
(c) amplification of the target nucleic acid utilising the extended EO.

20. A method of sequencing a target nucleic acid comprising
(a) hybridisation of an extendable oligonucleotide (EO) to a template oligonucleotide (TO), wherein the 5′ region of the TO
(i) overhangs the EO by at least one nucleotide; and
(ii) bears homology to the target nucleic acid; and
(b) extension of the EO such that at least one nucleotide complementary to the TO is added to the 3′ end of the EO; and
(c) dissociation of the annealed oligonucleotides and utilising the extended EO in a sequencing reaction.

21. A pair of oligonucleotides comprising an extendable oligonucleotide (EO) and a template oligonucleotide (TO) wherein
(a) the EO comprises a region complementary to a region of the TO;
(b) the EO is extendable at its 3′ end; and
(c) wherein the 5′ end of the TO is such that if the EO and TO were annealed, the 5′ end of the TO would overhang the 3′ end of the EO by at least one nucleotide.

22. A pair of oligonucleotides according to claim 21 wherein the at least one nucleotide is substantially similar to, or identical with, a nucleotide in a target nucleic acid.

23. A library comprising a plurality of pairs of oligonucleotides according to claim 22.

24. Two complementary libraries comprising, respectively, EOs and TOs, wherein the EOs and TOs are suitable for use in a method according to any one of claims 1 to 20.

25. A kit comprising a library of extendable oligonucleotides (EOs) and a complementary library of template oligonucleotides (TOs) wherein
(a) the EOs comprise a region complementary to a region of the TOs herein called a clamp;
(b) the EO is extendable at its 3′ end; and
(c) wherein the 5′ end of the TOs is such that when an EO from the library of EOs and a TO from the library of TOs are annealed, the 5′ end of the TO overhangs the 3′ end of the EO by at least one nucleotide.

26. A kit according to claim 25 wherein the clamp comprises a sequence motif useful for subsequent applications.

27. A kit according to claim 26 wherein the sequence motif is a recognition sequence for a restriction endonuclease, a phage polymerase transcription signal, a binding site for ribosomes, or a start codon enabling translation.

28. A kit according to claim 27 wherein the clamp is a region that is fully complementary between the EO and TO.

29. A method according to claim 1 wherein the TO includes a catch region comprising one or more degenerate or universal nucleotides.

30. A method according to claim 29 wherein the catch region lies between a constant 3′ region of the TO and a variable region.

31. A method according to claim 29 wherein the catch region is adjacent to, or forms part of, a clamp region as defined in claim 25.

32. A method according to claim 29 wherein the degenerate or universal positions of the catch region hybridise in most or all of its positions with most or all members of the EO library.

33. A method according to claim 1 wherein the nucleotides closest to the 3′ end of the EO are G or C.

34. A method according to any one of claims 1, 19 or claim 20 wherein the EO and TO comprise the following nucleotides:
EO: 5′ YYYYYXXXXX

|||||

TO: 3′ YYYYYNNNNNXXXXX
wherein the Y nucleotides are complementary, fixed nucleotides, and N, S and X are as herein defined.

35. A method according to claim 34 wherein the sequence of the TO is 3′YYYYYNNNSSXX)X 5′.

36. A pair of oligonucleotides according to claim 21 wherein the EO and TO comprise the following nucleotides:
EO: 5′ YYYYYXXXXX

|||||

TO: 3′ YYYYYNNNNNXXXXX
wherein the Y nucleotides are complementary, fixed nucleotides, and N, S and X are as herein defined.

37. A pair of oligonucleotides according to claim 36 wherein the sequence of the TO is 3′YYYYYNNNSSXXXXX 5′.

38. A kit according to claim 25 wherein the EOs and TOs comprise the following nucleotides:
EO: 5′ YYYYYXXXXX

|||||

TO: 3′ YYYYYNNNNNXXXXX
wherein the Y nucleotides are complementary, fixed nucleotides, and N, S and X are as herein defined.

39. A kit according to claim 38 wherein the sequence of the TOs is 3′YYYYYNNNSSXXXXX 5′.
